SEQUENCE LISTING (4 sequences)

SEQ ID NO: 1 - DNA, 13321 bp, Homo sapiens
  Homo sapiens mannose binding lectin 2 (MBL2)
acaccctctgaccacccaccccatggctataggggcctaaggtagtccttgccagatgcagtgcgcctgtgtatttgtgttggttaaaaagaaggcctgtgacatttacacattctcagagagttcatgagcaaaaacaagcaaagaaaaaatagcaggactggaacacaggacagtacaggatgaaaagtatgggaaggtcaggccttagggttacaaaggacactgctgacaacaagaaagcagcctaccgctgtcagctgtctctcacagcctgtctctcatggccagaccatggtattccctgctgaccctaaacacttccacagaataccggtgtacaacaatattactctgttgtgagatcccaaatcattccaaaatcatgtcttcatatagacagaaaaggttgctctgtgaaccacaaaaacgaaaaacatgcccctctcctgcttaatataaacatctgccactcttttacaaaatacagcttcgccttgctctgttctacttatctcctaaataaaagttaagatgcacaatcacagaattgcccagcttacggacagcaccctatccagaacagactcctgcttcttaaaccccttcccccaaaaccaactaaaacaaaacaagtcttatagaaatcccctcccgacaccctgttactgagaaatcctagggtacaccattgtgctcaatatccctgttggaacaaacagttaaaagaaactaaaaataataaatccattttatctgactataagcatattcctgatggtcctcacttggagagcaataacattctcattgctattgtcctactcacagcagctctgtggaaaagggaatgaggttcaaaatctgatttgaaagatgtaaaactgaggttcggccagaatatggggtctatctaagctcatattcatgagacaaaaccaagccttctggtttccaatctcaaaccatgcctctcacatggagccacccaagaacactgcttcccaagctgagaagactcttgaacagtcaccctggagggaatgaaggcctttcttaagcaggctgtcatggtgagccacgtgccatgcactctgaatgtggaccagttaaggacacaccactccacagccaaggcagcggtgccctctttcccagacgaggaaactgaggacatgaaggcagtggaacttgaataaggtcacactgttgggaagtgggagagctgggatttcaccctagtctacaaaaaaggggatgtagggcaccaggcagtcctcctgcaggtcagagaggcagttttctctgcagcctcttggggtgaggtggatttgttgtggcgctgccagaatggaagcagtgttaggaaagtggaaagttttttttttttttagacggagaggctctgtcgcccaggctggagcgcagtggtgcaatctcagctcactgcaagctccgcctcccgggctcactccattctcctgcctcagcctcctgggtagctgggattacaggcacccgccaccacgcccggctagtttgttgttttttttttttttgtattttttagtagagatggggtttcactgtgttagccaggatggtctcaatctcctgacctcatgatccgcccgcctcggcctcccaaggtgctgggattacaggagtgagccaccacgcctggctgggaagggttttaaagtccagaacaaatgcacacagtggccttatctatggggtggagtgcagagtgggcattcgtgtggagagagacattcaggctgcggcatgtggagagtggcctctccttttgggaaaggccttctggagagcaggatgtaactgggtgacactccaatctccggatcaattgctagattgactttcccccagatacccacaccctctgaacccagttctaggaaatttcagggtctggagagcagtttcctgacctctggactaggtaaacaatgaaagcaagcactgtgattaaagccagcaccgccagtgacctggagtcaactacctcacctcaccttctgccactggagcaggagcagggggaagctctggctgctaaagaataagaagggaaacaagtcttctcaggccctgtcttctttaggccctggatctcctgatatccactgatggctttaggcatgtggctctgtaccatcctggacatttttccaggcacagcagacaggtcagcaggaagtaaggaagccagtccctccacaagccaagaagacttcaattggaagggacctggaaatgcatcttactcacatttcttttggtgtttggaggcctccatagtacccccaccacatggccccgcagcctctgaacttctcaggatgggctccccaacaccccaagctacatggcatttttctgaaagccatatcagtaatgtagttgagaatcaaatgaactatgttcgtaaaagtattcttccatcccttctagattgtccccattaaccacagcaagatggttcaagccattaacacacacgatacccatgatcttatagtgccaagtgctgttcttttgcttcttatctcagttgagtggctcctagatagtcaagtcataaatgtgtgtgcccatgaaaacttcttattatcactcactattctagctaattctcaagtgcaatcaatacctcctgtttctcaagtgtgcctaccttcttaatctagctgccattaccctgacccaggccactgtcttctgctgcttagatcatggcaagcacttccttttagatctgcttttctccagtcctgttcccctcaacatcatagccagaacaagccttgcaaaattcaaatgtgatcacctctccactactgcagtccctctagtgccccctggaaggaaacccaaagtctttaacacagtttacaattcttgttatgaggatgaaatttgaagacattgtgctaagtgaaataagccagatacaagggggctattgtatgatttcacatatatgaggtacctaggatagatgaattcagagagacagaaagtaaaacagaagttaccagtggctgtgggtaggggtaaatggagaattctagtttaatgagttgagagtttcagtttgggatgatgaaaatcatcccatgatgtggtgatgatagcacaaccgtgtgaatgtacttaatgctactgagttgtgtgcttaataataatggtcaaaatggtaaattttatgtctattttaccacaatgaaaaactggaaaagtaaagtcaatgactataatatcataaagtgtaagtaacatgagagaaaataattcaggctaagaggatagagactgctaggtaggactattttagaataagtgatcaaggtactgatttctctgaaattggaatatttgaatagaagagaaagagctagctggcgatgcccgtaaggggtccaggcatggagacatcaaatgaagagtctgaggcaggaatggccttaatgcattaatggcagcaagaggtctatgtggtttcatggatgggtgtgtgcgtgcatgcacgtgtctgtgtgtatagttggtgtgcatgagcatgtgtatgcatttgtgagtatctttgtttttctgttggaaacaaggatgtcaacaggactaactaggtgaacctagctgtggtagttcagcaatgagatttgccaaaatggatggcaattgaaagaggaattgtgctgtattaaagcagacagctgccgagcactgtattggaggctcctaaatttctcatagttctgagaagagctgtttattaatctggagttgaaggccaggcccaggatatccttgttcacaaggttgattgggccctacattgctgagcccagcctcctccctcactctgaggcatctgccagagccccaggctagagggccagcgtccttgtcactgagtccctgctctgcagaaacaccagtaagtcatttctggtgaataaatctgggtttccggatcaagggtttggggagctgacatgaccctggaggcatggaggaggctttctaggggtggggggagtgggatggaaagcagaaatcaggttgaggatgtctctttttctgcttgtgctggagcatctgtgactttcctccattatctgttcatctgcagtggagactgtctttgttttcaaagggaaacttggaggcttagacctatggggctaggctgctgaggtttcttagggggcaatagctggaagaaagctctgaagaacaaatgaaaggttaatactgagaaatgggaggaggattcaaggcaagttttctaattgccagtggtttttgactcacagaacatggggaattcctgccagaaagtagagaggtatttagcactctgccagggccaacgtagtaagaaatttccagagaaaatgcttacccaggcaagcctgtctaaaacaccaaggggaagcaaactccagttaattctgggctgggttggtgactaaggttgaggttgatctgaggttgagaccttcctctttggatcaccagctttcagctcagggcctgccaatgagtaaatgatagttaacaggtcctggaggggaatcagctgcccagatacaaagatgggattcaggtggcagatggacccgaagaggacatggagagaaagaggaagctcctacagacacctgggtttccactcattctcattccctaagctaacaggcataagccagctggcaatgcacggtcccatttgttctcactgccaccgaaagcatgtttatagtcttccagcagcaacgccaggtgtctaggcacagatgaacccctccttaggatccccactgctcatcatagtgcctacctttgttaaagtactagtcacgcagtgtcacaaggaatgtttacttttccaaatccccagctagaggccagggatgggtcatctatttctatatagcctgcacccagattgtaggacagagggcatgctcggtaaatatgtgttcattaactgagattaaccttccctgagttttctcacaccaaggtgaggaccatgtccctgtttccatcactccctctccttctcctgagtatggtggcagcgtcttactcagaaactgtgacctgtgaggatgcccaaaagacctgccctgcagtgattgcctgtagctctccaggcatcaacggcttcccaggcaaagatgggcgtgatggcaccaagggagaaaagggggaaccaggtacgtgttgggctgttctgtctctgcaattctttaccttccagaggaaactgcctggggatatgaggagactgatgtcctatttgagtatatttttctcaactatactgtaactcaaaacagagattcagctctcattctctggcatctcagaattccacacagcagtttgtgactaatagttgtcttgccagcccaggaaagtggcccacaggtcaggccatcccgtgggacacaggatgaatttttcttctctgggtcattgtcatgtcagacccctattcacttcagtagggatggcaccaggttcaagaggccaaagaagagatggagtcagcaaacaaacataggttttactgggggaatctgtttacagggagatccagcagcagtgggctggacaggagaacaacaactactggtaaaaacaaatgcagttaattttcactttgcaccctccctgcagcaacctccacgtggcaactttatttcttaagttattgctctcaggtgcccaccatacagttattgagagcagtgctcagaaaggtcagtcctgggtcaaggtctcctttctcctgagaagggattgggcatcaaactcttgaagagagagagcaagaacatagatattaagtcacatttcctttgtcttccaacaggccaagggctcagaggcttacagggcccccctggaaagttggggcctccaggaaatccagggccttctgggtcaccaggaccaaagggccaaaaaggagaccctggaaaaagtccgggtaaggaccccagcaaggtctgagctgacttcacccagggttctgagaccttgagtatctggtaagaggtgccccttctcctgttccttcaaaggaagatacccaaatttgctttctgacccagtgccctcagccctctcttcattcttagagccttagggaattcctataagtgcctgagtacagacttacaaacttttcaagacaactccctcatcaggcactccctgtaagagctgagcctggattctccatcagctgaaacagtctaatgtgaggccctggtcctagagggagaaaacaggccatctaagtgtgggcttattatacctatttttacaaaagcatgtaaccatggtatgtcatattttacttcataagaaaccaagccaaagcttaatagcttaaaaacatagccgtcaatttaaatcacaagtctgtgggtcggccatgtgatttctctgctggtttaagctgggcttacccacatgtctgtggtcaactgatggccaatagtctcactcatgtgtctggctgttgtctgaatgttagctggagtgatgagggtaattgaaccacatctctcatcatccaacagggcttgttcacatagcagtgattacaggattcccaagagtagaaataagataggctttaggcttcttgaagactcagagcacattatcatttctgtcagactgtattggtcaaaacaagtcacaaggccagcccagattcaaggagtggggtaatagactcatgtcttaatgcgaggagctgtaccctattgaggccatttttgccatctactacaacaagtgatttgctcatgccctccccacttggataatgggcaactcacaaaaatcttggtttcctgactaacagaacaaacctggaaaaaacaaaaacaaaacaaaagacatcaaaagcaaaacctctccaaagacaaaaactctgcaatctttttcgtcactcttcaccaggtcataatagagtaacgcctcaagagatttctttttagttttgatagaacaatttatttgaaaaaaaaatctgtgaagaacctggcatatggattgctaggtaatcagggagtatatgtgtgcatgtgatatgtatgcgtactttgggatacagtaaacagacaaagagggacatttggggttggatggaaatagcgatttttcattctttgggcttttcagtattttttattttccttcaaagtgtaaacatcaatgtaaaataagctttttttttcagtttgaaaaagatactccactcccttgtatatgcacagttctgaggctccttctcaacaatgcttctttactttgttctaattttctagatggtgatagtagcctggctgcctcagaaagaaaagctctgcaaacagaaatggcacgtatcaaaaagtgtaagctttttctcttactctccaggcagcttgaagtttgggaaaaatagaatgcaacaaatatttgttgaatgcatataattttctgtaccctgctaggcatttctcatattcttacctcatgaaattctcacaacattttggtagaaatggaggcaaagggaagttaaattacttgttcaaatgcacagagctaataaatggcaggggtggtttatagatggaagtcagtctgactcgagagaccctaatcctttaccgtctgatattgctcactgaaaatgggacttatatccctttgttgcactggtattgagacctggccgtggggtctaacctgcctggggcaaatattttcagacattttttgtttggtctcaagttaacaatttagaattcagaagtccaaattatatgtcttttagaattctgatctgaaagcacagagaggcctttgtaccagtctgtctgttcacatttgggttgccatatttaacaaataaaaagacaaagcaccagttaaattggatttcagctgaaaaacatttttagtataagtgtgtctgaaaaattatatgggagatatactaaaatattcattgtttgtctgaaattcaaatttaactggacatactatattttatccggcaactgtactctagaagactttttcttgagaaataccttgagttgggcttaaggatgagtcagtttcacccactttttcacattttagggctcaccttctctctgggcaaacaagttgggaacaagttcttcctgaccaatggtgaaataatgacctttgaaaaagtgaaggccttgtgtgtcaagttccaggcctctgtggccacccccaggaatgctgcagagaatggagccattcagaatctcatcaaggaggaagccttcctgggcatcactgatgagaagacagaagggcagtttgtggatctgacaggaaatagactgacctacacaaactggaacgagggtgaacccaacaatgctggttctgatgaagattgtgtattgctactgaaaaatggccagtggaatgacgtcccctgctccacctcccatctggccgtctgtgagttccctatctgaagggtcatatcactcaggccctccttgtctttttactgcaacccacaggcccacagtatgcttgaaaagataaattatatcaatttcctcatatccagtattgttccttttgtgggcaatcactaaaaatgatcactaacagcaccaacaaagcaataatagtagtagtagtagttagcagcagcagtagtagtcatgctaattatataatatttttaatatatactatgaggccctatcttttgcatcctacattaattatctagtttaattaatctgtaatgctttcgatagtgttaacttgctgcagtatgaaaataagacggatttatttttccatttacaacaaacacctgtgctctgttgagccttcctttctgtttgggtagagggctcccctaatgacatcaccacagtttaataccacagctttttaccaagtttcaggtattaagaaaatctattttgtaactttctctatgaactctgttttctttctaatgagatattaaaccatgtaaagaacataaataacaaatctcaagcaaacagcttcacaaattctcacacacatacatacctatatactcactttctagattaagatatgggacatttttgactccctagaagccccgttataactcctcctagtactaactcctaggaaaatactattctgacctccatgactgcacagtaatttcgtctgtttataaacattgtatagttggaatcatattgtgtgtaatgttgtatgtcttgtttactcagaattaagtctgtgagattcattcatgtcatgtgtacaaaagtttcatccttttcattgccatgtagggttcccttatattaatattcctcagttcatccattctattgttaataggcacttaagtggcttccaatttttggccatgaggaagagaacccacgaacattcctggacttgtcttttggtggacatggtgcactaatttcactacctatccaggagtggaactggtagaggatgaggaaagcatgtattcagctttagtagatattaccagttttcctaagtgattgtatgaatttatgctcctaccggcaatgtgtggcagtcctagatgctctatgtgcttgtaaaaagtcaatgttttcagttctcttgattttcattattcctgtggatgtaaagtgatatttccccatggttttaatctgtatttccccaacatgtaataaggttgaacacttttttatatgcttattgggcacttgggtatcttcttttgtgaagtacccgttcacatttttgtattttgtttaaattagttagccaatatttttcttactgatttttaagttatttttacattctgaatatgtcctttttaatgtgtattacaaatattttgctagtttttgacttgctcctaatgttgaattttgatgaacaaaatttcctaattttgagaaagtcttatttattcatattttctttcaaaattagtgctttttgtgtcatgtttaagaaatttttgcccatcccaaaatcataagatatttttcatgattttgaaaccatgaagagatttttcatgattttgaaatcatgaagatatttttccatttttttctaatagttttattaataaacattctatctattcctggtagaatagatatccacttgagacagcactatgtaggaaagaccatttttcctccactgaactagggtggtgcatttttgtaagttaggtaactgtatgtgtgtgtgtctgtttctgggctgtctattctagtctatttgttgatgcttgtgtcaaacagtacactatcttaattattgtacatttatagttgtaactatagtccagctttgttcttcttaaagtcaagatttccatataaatattagaaacagcttctcaatttctacaaaatcctgatgaggtttctactgggaccacattgagtctatcaatcaacttatgcagaactggcaacttactactgaatctctaatcaatgttcatcatgtatcgcttcatgtaactagaatttctttaacttaattgctatgttttgacatttttagtttaaaaaccttgtatatcttgttttggtggttttagtgattttaataatatattttaaatattttttcttttctattgttgtacacagaaatacagttaagttttgtgtgtagtcttacgatgtttagtaaactcaataagtttatttcttaaatctagtaatttgtagattcctctggattttgtatatgcatagtcatgtaagctgaaaatatggcaatacttgcttcttcccaattgctttaccttttttcttaccttattgcactggttagcaaccccaatacagagaccaccagatcaggtatagactcctgaaagacaatataatgaagtgctccagtcaggcctatctaaactggattcacagctctgtcacttaattgctacatgatctagagccagttactttgtgtttcagccatgtatttgcagctgagagaaaataatcattcttatttcatgaaaattgtggggatgatgaaataagttaacacctttaaagtgtgtagtaaagtatcaggatactatattttaggtcttaatacacacagttatgccgctagatacatgctttttaatgagataatgtgatattatacataacacatatcgatttttaaaaattaaatcaaccttgctttgatggaataaactccatttagtcacattttatcttgtgtatgtattattggagtggacttgttaatagtttgtttagaacgtttgcacctatgttcatgagagatactagctttttttcccctccttaatgcacttgctatggtttggggtcaatgttatgtaagcctcattaaaatgagctgtgatgtgctctcatgctccttattatctaaaataatttgtgtaaaattatactttaatgttagataaattcaccagtgaagccatcttggaatggtgttttctgttgggaaatgattttaatttcagatttaatttcctcatgatctaaacctattcatgttttctgtcttttgttcggtttcagtgagttatatttttcaatgaatgtgtcaatttcctctgaatatatatgtattggcataaaattgttacaatatctttgtatttcttttcaatataagtaggatctatagcaatgtctatgtatctgttatgattatttgcattttatctttattttattgtatttttttttgagatggagtctcactctgtcacccaggctggagtggctggagtacagtggcacgatcttggctcactgcaatctctgcctcccaggttcaagagattctcctgcctcagcctcccaggtagctgggattacagatgcccaccaccattcctggctaatttttttaattttttgtggagacgaggtttcgccatgctggctaggctggtctcaaacccctgacctgaggtgatctacctgcctcagcctcccaaagtgctgggattacaggcatgagccactgtgcttggcccatctctttttttcttaatcaatcttgcaaaggatttgtccattttattaaatttttccaaaaacttctttttggtgtttactaacctctatgattacatttatttatatctgttcctgtctttatcatttcttgtatcgatcttctgtgggtttaattggccattctttttctagcttcttttaattgacatttagagaactgattttcttccttccttctttgctaatataatcatttaaagttataatttttatttaagaactatttcagctgcataccacaggtttggatatgtatcttttcccaccaattcaaaataggtttttcatatccaattcaaaatattttataattttcattgtgacttcttcgactcatggtttatttataagtgtgtggtttaattcctaaatatttgagtattttaaaaagctatactcctgtaagtgatttatagtttaattccactgtatccagaaatatacttttatatgagttcaatcttttgaaatttgttaagattttattttatgatccagtttatgatttagaatggtaaatgtattatgtgcactagaaataatgtggtttttacagttgattagtgtatcattaggtcaggatatgaatcttctttgtattttctgtattctcactaatattttgcccttttagaaggaaataacaaagatcagaatagaaataaatgacatagagagtagaaaagcaaatagatcaatgaaactcaaaagttaatttttgtgaaatgatacaatttccaaactttagctaaaataactaagaaaaatacagagaagactcaaaatcagaaatgagaggagacacgacaactgataacatacaagtacaaaggatcataagagctacattcctttgtgttacattgttttctctttacattctgattgtctctcgcttgctcgctgtcttcttttaaagttttgtttcagaagaactaaaaaaatctaggtttaaaatgtacagatatcctttgtaacttagcattaggcaaaattgtcttagagaactcaattacttttcaagtaaaaaatgataaattgaatttcatcaaaattaaagattaactcagtttgttaaacattaggaaaacaggcaaaccatgactaggagaaaaatatttgcaaatattggcctgcatctgtcaaaggat

SEQ ID NO: 2 - protein, 248 aa, Homo sapiens
  Primary amino-acidic sequence of MBL
MSLFPSLPLLLLSMVAASYSETVTCEDAQKTCPAVIACSSPGINGFPGKDGRDGTKGEKGEPGQGLRGLQGPPGKLGPPGNPGPSGSPGPKGQKGDPGKSPDGDSSLAASERKALQTEMARIKKWLTFSLGKQVGNKFFLTNGEIMTFEKVKALCVKFQASVATPRNAAENGAIQNLIKEEAFLGITDEKTEGQFVDLTGNRLTYTNWNEGEPNNAGSDEDCVLLLKNGQWNDVPCSTSHLAVCEFPI

SEQ ID NO: 3 - DNA, 13 bp, Homo sapiens
atccccgcattga

SEQ ID NO: 4 - DNA, 28 bp, Homo sapiens
agatccccgcgcgtgcaacggctgcgga

(19) United States
(12) Patent Application Publication
(10) Pub. No.: US 2024/0123029 A1
(43) Pub. Date: Apr. 18, 2024

(54) COMPOSITIONS AND METHODS FOR TREATING AND PREVENTING CORONAVIRUS INFECTIONS

(71) Applicants: HUMANITAS MIRASOLE S.P.A., Rozzano(MI) (IT); HUMANITAS UNIVERSITY, Pieve Emanuele (MI) (IT); OSPEDALE SAN RAFFAELE S.R.L., Milano (MI) (IT)

(72) Inventors: Alberto MANTOVANI, Rozzano (IT); Cecilia GARLANDA, Pieve Emanuele (IT); Barbara BOTTAZZI, Rozzano (IT); Elisa VICENZI, Milano (IT)

(21) Appl. No.: 18/275,776

(22) PCT Filed: Feb. 8, 2022
(86) PCT No.: PCT/EP2022/052944

§ 371 Date: Aug. 3, 2023

(30) Foreign Application Priority Data
Feb. 8, 2021 (IT) 102021000002738
Dec. 14, 2021 (EP) 21214373.9

(51) Int. Cl. A61K 38/17; A61K 45/06; A61P 31/14; C12Q 1/70

(52) U.S. Cl. CPC A61K 38/178; A61K 45/06; A61P 31/14; C12Q 1/701; C12Q 2600/156; C12Q 2600/172

(57) ABSTRACT

The present invention relates to a molecule for use in the treatment and/or prevention of viral infections caused by highly pathogenic coronaviruses, including Severe Acute Respiratory syndrome Coronavirus 2 (SARS-COV-2), or variants thereof, Severe Acute Respiratory syndrome Coronavirus ((SARS)-COV) and sarbecoviruses, said molecule being a mannose binding lectin (MBL) polypeptide, or a functional fragment, derivative, mutein or variant thereof, or an homologue having a percentage of identity with MBL polypeptide of at least 50, 60, 70, 80 or 90%, preferably for use in the treatment and/or prevention of 2019 Coronavirus disease (COVID-19).

### TECHNICAL FIELD

The present invention relates to a molecule for use in the treatment and/or prevention of viral infections caused by highly pathogenic coronaviruses, including Severe Acute Respiratory syndrome Coronavirus 2 (SARS-COV-2), Severe Acute Respiratory syndrome Coronavirus ((SARS)-CoV) and pre-pandemic sarbecoviruses said molecule being a mannose binding lectin (MBL) polypeptide, or a functional fragment, derivative, mutein or variant thereof, or an homologous having a percentage of identity with MBL polypeptide of at least 50, 60, 70, 80 or 90%, preferably for use in the treatment and/or prevention of 2019 Coronavirus disease (COVID-19).

### BACKGROUND ART

Two highly pathogenic coronaviruses, Severe Acute Respiratory syndrome Coronavirus ((SARS)-CoV) and Severe Acute Respiratory syndrome Coronavirus 2 (SARS-COV-2) have emerged in the last 20 years causing SARS in 2002-2003 and Coronavirus disease-2019 (COVID-19) in 2020². Both viruses had spread globally, however, SARS-COV stopped to circulate in the early summer of 2003 whereas SARS-COV-2 is currently spreading worldwide causing an unprecedented challenge. The SARS-COV-2 emergence has highlighted the need of antivirals that are effective in inhibiting viral invasion and replication in target cells. In this context, the innate immune system represents a first line of resistance against viruses, bacteria and protozoa. Innate immunity includes both a cellular and a humoral arm. Components of the heterogeneous cellular arm of the innate immunity include myeloid cells and innate lymphoid cells. No less important in antimicrobial resistance is the humoral arm of innate immunity^{5.7}. Humoral innate immunity consists of a limited, but diverse, set of molecular families such as Complement components (C1q), ficolins, mannose binding lectin (MBL) and pentraxins [C-reactive protein (CRP), Serum amyloid P component (SAP), pentraxin 3 (PTX3)]^{5.7} that can be considered as functional ancestors of antibodies for their capacity to directly interact with pathogens. The role of the humoral arm of innate immunity in COVID-19 resistance and pathogenesis is still unclear. WO2006046786 discloses a method for production of multimeric recombinant human MBL and its possible use for the treatment of viral infections.

The work of Chatterjee et al. (2020)⁷² reviews therapeutic strategies for the treatment of COVID-19.

In the work of Klaassen et al. (2020)⁷³ coding regions of MBL2 gene are analyzed for their effect in the response to Sars-Cov-2 infection.

It is still felt the need of a therapeutic agent able to treat COVID-19 patients.

### SUMMARY OF THE INVENTION

Inventors have herein found that MBL (Mannose-Binding Lectin) interacts with SARS-COV-2 Spike protein (active trimer) through its carbohydrate recognition domain (CRD), inhibiting infection of respiratory cells (Calu-3) and viral replication. The interaction is Ca²⁺-dependent and occurs between the MBL lectin domain and the glycosidic sites exposed by the Spike protein. MBL inhibits in a concentration-dependent manner the viral replication and cytopathic effects of SARS-COV-2 in human respiratory cell line.

Furthermore, it has been found that MBL binds trimeric Spike, including that of variants of concern, in a glycan-dependent way and inhibited SARS-COV-2 in three in vitro models. Moreover, upon binding to Spike, MBL activated the lectin pathway of complement activation. It was also found that genetic polymorphisms at the MBL locus are associated with disease severity.

These results demonstrate the anti-SARS-COV-2 activity of MBL and the therapeutic potential of this component of the humoral innate immunity.

### DETAILED DESCRIPTION OF THE INVENTION

The present invention relates to a molecule for use in the treatment and/or prevention of viral infections caused by highly pathogenic coronaviruses, including Severe Acute Respiratory syndrome Coronavirus 2 (SARS-COV-2), Severe Acute Respiratory syndrome Coronavirus ((SARS)-COV) and sarbecoviruses, said molecule being a mannose binding lectin (MBL) polypeptide, or a functional fragment, derivative, mutein or variant thereof, or an homologue having a percentage of identity with MBL polypeptide of at least 50, 60, 70, 80 or 90%, preferably for use in the treatment and/or prevention of 2019 Coronavirus disease (COVID-19).

Preferably the polypeptide MBL is a molecule comprising or consisting of a sequence having at least 95% identity with the sequence encoded by SEQ ID No. 1.

Preferably the polypeptide MBL is a molecule comprising or consisting of a sequence having at least 95% identity with the sequence of SEQ ID No. 2.

A further object of the invention is a nucleic acid molecule coding for the molecule as defined above for use in the treatment and/or prevention of viral infections caused by highly pathogenic coronaviruses, including Severe Acute Respiratory syndrome Coronavirus 2 (SARS-COV-2), Severe Acute Respiratory syndrome Coronavirus ((SARS)-COV) and sarbecoviruses, preferably for use in the treatment and/or prevention of the 2019 coronavirus disease (COVID-19).

Within the meaning of the present invention, for “a nucleic acid molecule coding for” it is intended a nucleic acid able to code for said polypeptide, fragment, derivative or variant thereof, i.e. a nucleic acid that in a suitable system, such as in a cell, allows to obtain said protein. Such nucleic acid can be any kind of nucleic acid, in particular it can be a genomic gene, a DNA stretch, a recombinant DNA, a RNA, such as a mRNA, or a cDNA.

In a particular embodiment, a mRNA coding for the MBL polypeptide, or a functional fragment, derivative, mutein or variant, or an homologue thereof is used.

In an embodiment, the nucleic acid coding for the MBL polypeptide is a molecule comprising or consisting of a sequence having at least 95% identity with the sequence of SEQ ID No. 1.

Another object of the invention is a vector comprising said nucleic acid molecule or expressing said MBL molecule for use in the treatment and/or prevention of viral infections caused by highly pathogenic coronaviruses, including Severe Acute Respiratory syndrome Coronavirus 2 (SARS-CoV-2), Severe Acute Respiratory syndrome Coronavirus ((SARS)-COV) and sarbecoviruses, preferably for use in the treatment and/or prevention of the 2019 coronavirus disease (COVID-19).

A further object of the invention is a genetically engineered host cell able to express the molecule as defined above or comprising a vector as defined above for use in the treatment and/or prevention of viral infections caused by highly pathogenic coronaviruses, including Severe Acute Respiratory syndrome Coronavirus 2 (SARS-COV-2), Severe Acute Respiratory syndrome Coronavirus ((SARS)-COV) and sarbecoviruses, preferably for use in the treatment and/or prevention of the 2019 coronavirus disease (COVID-19), preferably said cell being transformed with a vector as defined above.

An object of the invention is a pharmaceutical composition comprising at least one molecule as defined above or at least one nucleic acid molecule as defined above or the vector as defined above, or at least one host cell as defined above and at least one pharmaceutically acceptable excipient and/or carrier, for use in the treatment and/or prevention of viral infections caused by highly pathogenic coronaviruses, including Severe Acute Respiratory syndrome Coronavirus 2 (SARS-COV-2), Severe Acute Respiratory syndrome Coronavirus ((SARS)-COV) and sarbecoviruses, preferably for use in the treatment and/or prevention of the 2019 coronavirus disease (COVID-19).

Preferably, the pharmaceutical composition further comprises at least one therapeutic agent, such as an antiviral therapy and/or wherein said pharmaceutical composition is in a formulation for local administration, preferably selected from the group consisting of pulmonary delivery by dry powder formulations or by nebulization of liquid formulations, or systemic administration. For sarbecoviruses (SARS Betacoronavirus) it is intended the subgenus of Betacoronavirus genus. Examples of sarbecoviruses are severe acute respiratory syndrome-related coronavirus (SARSr-COV or SARS-COV), such as Bat SARS-like coronavirus RsSHC014, or SHC014-CoV, and Il Bat SARS-like coronavirus WIVI, or Bat SL-CoV-WIV1. Preferably it is intended sarbecoviruses known before the discovery of SARS-COV-2.

In the present invention, COVID-19 is preferably caused by severe acute respiratory syndrome coronavirus 2 (SARS-COV-2) or its variants, particularly the 501 or 614 variants or other more infectious variants, or any variant comprising mutations in the gene encoding SARS-COV-2 Spike protein. For example, a variant selected from the group consisting of: the B.1.1.7 variant or a variant, the B.1.1.28 or P.1 variant or γ variant, the B.1.351 variant or β variant, the B.1.617.2 variant or δ variant and the Omicron (o) variant or B.1.1.529 variant.

In the context of the present invention, in an embodiment the subject to be treated presents a genetic polymorphism in the MBL gene leading to a low MBL production.

In an embodiment, the subject to be treated has one or more of the following Single Nucleotide Polymorphisms (SNPs) in the MBL gene: rs5030737, rs1800450, rs1800451, rs150342746, rs10824845 and rs11816263. Preferably, said SNP is present on both the gene alleles.

In an embodiment, the subject to be treated has at least one of the following haplotypes in the MBL gene:

ATCGCAA
CCC
TCCCC
TCAGACC
TA
[SEQ ID N. 3]
ATCCCCGCATTGA
[SEQ ID N. 4]
AGATCCCCGCGCGTGCAACGGCTGCGGA,

which are characterized by the SNPs described in the following Table 1:

| Haplotype | SNPs |
|---|---|
| ATCGCAA | 6 SNPs, rs11344513\|rs7071467 |
| CCC | 3 SNPs, rs17662822\|rs1159798 |
| | rs1912619 |
| TCCCC | 5 SNPs, rs2204344\|rs12218074 |
| | rs80035245\|rs7935712\| |
| | rs10824836 |
| TCAGACC | 5 SNPs, rs16935439\|rs147096903\| |
| | rs10824839\|rs11003267\|rs11003268 |
| TA | 2 SNPs, rs10824844\|rs10824845 |
| ATCCCCGCATTGA | 9 SNPs, rs57504125\| |
| [SEQ ID N. 3] | chr10:5308418:G:A |
| AGATCCCCGCGC | 24 SNPs, rs71032688\|rs7092597 |
| GTGCAACGGCTG | |
| CGGA | |
| [SEQ ID N. 4] | |

wherein the number of SNPs composing the haplotype is indicated; all the SNPs forming the haplotype are shown for short haplotypes (including max 5 SNPs) while for more complex haplotypes (including more than 5 SNPs) only the first and the last SNPs are indicated.

Preferably the subject has the TA haplotype which is composed of the SNPs rs10824844 and rs 10824845.

It is also an object of the invention, a method for the prognosis of a Coronavirus disease, preferably 2019 Coronavirus disease (COVID-19), comprising determining the presence or the absence in an isolated biological sample from a subject of at least one of the above mentioned haplotypes in the MBL gene, i.e. ATCGCAA, CCC, TCCCC, TCAGACC, TA, ATCCCCGCATTGA [SEQ ID N.3], AGATCCCCGCGCGTGCAACGGCTGCGGA [SEQ ID N.4], wherein preferably if at least one of said haplotype is identified the subject is at risk of short-term mortality and/or of being affected by a more severe disease and/or of a poor prognosis. It is also an object of the invention, a method for the prognosis of a Coronavirus disease, preferably 2019 Coronavirus disease (COVID-19), comprising determining the presence or the absence in an isolated biological sample from a subject of at least one of the following SNPs in the MBL gene: rs5030737, rs1800450, rs1800451 rs150342746, rs10824845 and rs11816263, wherein preferably if at least one of said SNPs is identified the subject is at risk of short-term mortality and/or of being affected by a more severe disease and/or of a poor prognosis.

Preferably, if the subject has two alleles of at least one of the following SNPs: rs5030737, rs1800450, and rs1800451, the subject is at risk of short-term mortality and/or of being affected by a more severe disease and/or of a poor prognosis.

It is also an object of the invention, a method for the prognosis of a Coronavirus disease, preferably 2019 Coronavirus disease (COVID-19), comprising determining the presence or the absence in an isolated biological sample from a subject of the MBL gene haplotype CCGGCC, said haplotype consisting of the following SNPs: rs1800451, rs1800450, rs5030737, rs7095891, rs7096206, rs11003125, wherein preferably if said haplotype is identified the subject is less at risk of short-term mortality and/or of being affected by a more severe disease and/or of a poor prognosis. For “less at risk” it is intended that the subject has a lower risk with respect to the risk of the general population.

Preferably, the biological sample is selected from the group consisting of: plasma, serum, blood, CSF, saliva, or Bronchoalveolar lavage fluid (BALF), pulmonary tissue.

Preferably, the subject is a patient who has been diagnosed with a Coronavirus disease, in particular COVID-19 or is a subject at risk of contracting or developing a Coronavirus disease.

Preferably, the Coronavirus is SARS-COV-2, and/or the Coronavirus disease is selected from the group consisting of: Severe Acute Respiratory Syndrome (SARS), Middle East Respiratory Syndrome (MERS), COVID-19, coronavirus-associated acute respiratory distress syndrome (ARDS).

Determining the presence or the absence of the mentioned haplotypes and/or SNPs in an isolated biological sample from a subject can comprise isolation and extraction of the DNA from the sample and SNP array genotyping for the identification of the indicated SNPs in the MBL gene. These techniques can be carried out by the skilled person according to the general knowledge in the field. In particular, extraction and sequencing of DNA to identify the presence or the absence of the indicated SNPs such as manual or automatic methods to elaborate sequencing data and define the haplotypes can be carried out accorging to the general knowledge in the field.

Preferably the molecule or the at least one nucleic acid molecule or the vector or the host or the pharmaceutical composition as defined above is for use in the early stages of the disease.

In the context of the present invention the term “MBL” includes the polynucleotide (e.g. the gene or the transcript) and the polypeptide (or protein) thereof. “MBL2” is herein used as a synonymous for the MBL gene.

The term polynucleotide and polypeptide also includes derivatives and functional fragments thereof.

The polynucleotide may be synthesized using oligonucleotide analogs or derivatives (e.g., inosine or phosphorothioate nucleotides).

In the context of the present invention, the genes/proteins as above defined are preferably characterized by the sequences identified by their NCBI Gene ID and Gen Bank Accession numbers. Preferably, MBL is characterized by the sequences disclosed in GenBank: CAB56121.1 (GenBank release of Oct. 15, 2021), NCBI Reference Sequence: NG_008196.1 (GenBank release of Oct. 15, 2021).

The term gene herein also includes corresponding orthologous or homologous genes, isoforms, variants, allelic variants, functional derivatives, functional fragments thereof.

The expression “protein” or “polypeptide” is intended to include also the corresponding protein encoded from a corresponding orthologous or homologous genes, functional mutants, functional derivatives, functional fragments or analogues, isoforms thereof.

In the context of the present invention, the term “polypeptide” or “protein” includes:
i. the whole protein, allelic variants and orthologs thereof;
ii. any synthetic, recombinant or proteolytic functional fragment;
iii. any functional equivalent, such as, for example, synthetic or recombinant functional analogues.

In the context of the present invention the term “comprising” also includes the terms “having essentially” or “consisting essentially”.

In the present invention, the herein mentioned “protein(s)” or “polypeptide(s)” also comprises the protein encoded by the corresponding orthologous or homologous genes, functional mutants, functional derivatives, functional fragments or analogues, isoform, splice variants thereof.

In the present invention “functional” is intended for example as “maintaining their activity”.

As used herein “fragments” refers to polypeptides having preferably a length of at least 10 amino acids, more preferably at least 15, at least 17 amino acids or at least 20 amino acids, even more preferably at least 25 amino acids or at least 37 or 40 amino acids, and more preferably of at least 50, or 100, or 150 or 200 amino acids.

In an embodiment, the MBL binding sites on the Spike protein of SARS-COV-2 are N603, N801 and N1074 or N603, N1074 and N709.

It is within the scope of the invention a host cell comprising the nucleic acid as defined above, or the vector as defined above.

The terms “host cell”, “host cell line”, and “host cell culture” are used interchangeably and refer to cells into which an exogenous nucleic acid has been introduced, including the progeny of such cells. The host cells include “transformants” and “transformed cells,” which include the transformed primary cell and the progeny derived therefrom, without taking into account the number of steps. The progeny may be not completely identical in nucleic acid content to a parent cell, but may contain mutations. In the present invention mutant progenies are included, which have the same function or biological activity as that for which they have been screened or selected in the originally transformed cell. The nucleic acids of the invention can be used to transform a suitable mammalian host cell. Mammalian cells available as expression hosts are well known and include, for example, CHO and BHK cells. Prokaryotic hosts include, for example, E. coli, Pseudomonas, Bacillus, etc. Antibodies of the invention can be fused to additional amino acid residues, such as tags that facilitate their isolation. The term “vector”, as used in the present invention refers to a nucleic acid molecule capable of propagating another nucleic acid to which it is linked. The term includes the vector as a self-replicating nucleic acid structure as well as the vector incorporated into the genome of a host cell in which it was introduced. Certain vectors are capable of directing the expression of nucleic acids to which they are operably linked. In the present such vectors are referred to as “expression vectors.” Any suitable expression vector can be used, for example prokaryotic cloning vectors such as plasmids from E. coli, such as colE1, pCR1, pBR322, pMB9, pUC. Expression vectors suitable for expression in mammalian cells include derivatives of SV-40, adenovirus, retrovirus-derived DNA sequences. The expression vectors useful in the present invention contain at least one expression control sequence that is operatively linked to the sequence or fragment of DNA that must be expressed.

The highly pathogenic coronaviruses as above defined comprise also those emerging in the future and expressing a conserved Spike protein.

In an embodiment, the viral infection is selected from the group consisting of: Severe Acute Respiratory Syndrome (SARS), Middle East Respiratory Syndrome (MERS), COVID-19, coronavirus-associated acute respiratory distress syndrome (ARDS).

In a preferred embodiment, the Coronavirus is SARS-COV-2.

Preferably the severe acute respiratory syndrome coronavirus 2 (SARS-COV-2) is characterized by the sequence disclosed with the Genome Reference Sequence (NC_045512), NCBI Reference Sequence: NC_045512.2 (GenBank release of Oct. 15, 2021).

The viral variants above defined are for example:
GR/501Y.V1 (B.1.1.7): UK
GH/501Y.V2 (B.1.351): South Africa
GR/484K.V2 (B.1.1.28): Brazil
GH/452R.V1 (B.1.429): USA (CA)
B.1.617.2: India
B.1.1.529: Omicron (o)

### See e.g.:

doi: https://doi.org/10.1101/2020.12.21.20248640
https://www.who.int/csr/don/31-december-2020-sars-cov2-variants/en/
https://cov-lineages.org/global_report_B.1.1.7.html
https://cov-lineages.org/global_report_B.1.351.html
https://cov-lineages.org/global_report_P.1.html

Genetic polymorphism in the MBL gene leading to a low MBL production are known to the expert in art and are e.g. disclosed in Journal of Infectious Diseases 2005; 191:1697-704 and in Heitzeneder et al., Clin Immunol 2012, 143:22-38. Exemplary SNPs are:
NM_000242.3(MBL2):c. 170G>A (p.Gly57Glu)
NM 000242.2(MBL2):c.161G>A (p.Gly54Asp)
NM_001378373.1(MBL2):c.154C>T (p.Arg52Cys).

It has now been found that some SNPs in the MBL gene have a significant predisposing effect to COVID-19, in particular the following SNPs: rs5030737, rs1800450, rs1800451 rs150342746, rs10824845 and rs11816263, more in particular in biallelic conditions.

More in particular, a significant predisposing effect was observed in those individuals carrying two disruptive alleles among rs5030737, rs1800450, and rs1800451.

Also, it has been found that 7 haplotypes in the MBL gene are strongly associated with severe COVID-19, i.e .: ATCGCAA, CCC, TCCCC, TCAGACC, TA, ATCCCCGCATTGA [SEQ ID N. 3] and AGATCCCCGCGCGTGCAACGGCTGCGGA [SEQ ID N. 4]. Details of each haplotype and of the SNPs forming the haplotypes are in the Table 1 above wherein the number of SNPs composing each haplotype is indicated. All the SNPs forming the haplotype are shown for short haplotypes (including max 5 SNPs). For more complex haplotypes (including>5 SNPs) only the first and the last SNPs are indicated.

It was also found that MBL gene haplotype CCGGCC has a protective effect on COVID-19 disease.

It is a further object of the invention a pharmaceutical composition comprising at least one molecule as defined above and pharmaceutical acceptable excipients, preferably said composition being for use by local administration or parenteral administration, in particular intravenously. The composition comprises an effective amount of the molecule. The pharmaceutical compositions are conventional in this field and can be produced by the skilled in the art just based on the common general knowledge. The formulations useful in therapy as described herein may e.g. comprise the molecule as described above, in a concentration from about 0.1 mg/ml to about 100 mg/ml, preferably from 0.1 to 10 mg/ml, more preferably from 0.1 to 5 mg/ml. In other formulations, the molecule concentration may be lower, e.g. at least 100 pg/ml. The molecule of the invention is administered to the patient in one or more treatments. Depending on the type and severity of the disease, a dosage of e.g. about 1 mg/kg to 20 mg/kg of the molecule may be administered, for example in one or more administrations, or by continuous infusion.

Any method of administration may be used to administer the molecule of the present invention, in particular, for example, the administration is by local administration, such as pulmonary delivery by dry powder formulations or by nebulization of liquid formulations, or by sistemic administration. In particular embodiments, the pharmaceutical composition of the present invention can be administered in the form of single dosage (for example, tablet, capsule, bolus, etc.). For pharmaceutical applications, the composition may be in the form of a solution, for example, of an injectable solution, emulsion, suspension, or the like. The vehicle can be any vehicle suitable from the pharmaceutical point of view. Preferably, the vehicle used is capable of increasing the entry effectiveness of the molecules into the target cell. In the pharmaceutical composition according to the invention, the molecule may be associated with other therapeutic agents, such as antiviral agents.

The term “pharmaceutical composition” refers to a preparation that is in such a form as to permit to the biological activity of an active ingredient contained therein to be effective, and which contains no additional components which are unacceptably toxic to a subject to which the formulation may be administered.

Optimal pharmaceutical compositions can be determined by one skilled in the art depending upon, for example, the intended route of administration, delivery format and desired dosage. See, for example, REMINGTON'S PHARMACEUTICAL SCIENCES, Id. Such compositions may influence the physical state, stability, rate of in vivo release and rate of in vivo clearance of the antibodies of the invention. Administration routes for the pharmaceutical compositions of the invention include orally, through injection by intravenous, intraperitoneal, intramuscular, intravascular, intraarterial, intraportal; by sustained release systems or by implantation devices. The pharmaceutical compositions may be administered by bolus injection or continuously by infusion, or by implantation device. The pharmaceutical composition also can be administered locally via implantation of a membrane, sponge or another appropriate material onto which the desired molecule has been absorbed or encapsulated. Where an implantation device is used, the device may be implanted into any suitable tissue or organ.

Also within the context of the invention are polypeptides and nucleic acids which are homologous to or substantially identical with, based on sequence, to a polypeptide or nucleic acid of the invention and retain the relevant function.

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. An “unrelated” or “non-homologous” sequence shares less than 40% identity, though preferably less than about 25% identity, with a nucleic acid sequence of the present invention.

Substantially complementary nucleic acids are nucleic acids in which the complement of one molecule is substantially identical to the other molecule. Two nucleic acid or protein sequences are considered “substantially identical” if, when optimally aligned, they share at least about 70% sequence identity. In alternative embodiments, sequence identity may for example be at least 75%, at least 80%, at least 85%, at least 90%, or at least 95%. Optimal alignment of sequences for comparisons of identity may be conducted using a variety of algorithms, such as the local homology algorithm of Smith and Waterman, 1981, Adv. Appl. Math 2. 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, WI, U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11 , the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO4, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1 , Green Publishing Associates, Inc., and John Wiley & Sons, Inc., New York, at p. 2.10.3). Alternatively, hybridization to filter-bound sequences under stringent conditions may, for example, be performed in 0.5 M NaHPO4, 7% SDS, 1 mM EDTA at 65<0>C, and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 Overview of principles of hybridization and the strategy of nucleic acid probe assays”, Elsevier, New York). Generally, stringent conditions are selected to be about 5[deg. ]C. lower than the thermal melting point for the specific sequence at a defined ionic strength and pH.

The term “vector” is commonly known in the art and defines e.g., a plasmid DNA, phage DNA, viral DNA and the like, which can serve as a vehicle into which the nucleic acid of the present invention can be cloned. Numerous types of vectors exist and are well known in the art. In a particular embodiment, the vector is a viral vector which can introduce a molecule, e.g. a chimeric co-stimulatory molecule, in a cell or in a living organism. In an embodiment the viral vector is a retroviral vector, a lentiviral vector, an adenoviral vector or an adeno-associated vector (AAV). Various genes and nucleic acid sequences of the invention may be recombinant sequences. The term “recombinant” means that something has been recombined, so that when made in reference to a nucleic acid construct the term refers to a molecule that is comprised of nucleic acid sequences that are joined together or produced by means of molecular biological techniques. The term “recombinant” when made in reference to a protein or a polypeptide refers to a protein or polypeptide molecule which is expressed using a recombinant nucleic acid construct created by means of molecular biological techniques. The term “recombinant” when made in reference to genetic composition refers to a gamete or progeny or cell or genome with new combinations of alleles that did not occur in the parental genomes. Recombinant nucleic acid constructs may include a nucleotide sequence which is ligated to, or is manipulated to become ligated to, a nucleic acid sequence to which it is not ligated in nature, or to which it is ligated at a different location in nature. Referring to a nucleic acid construct as “recombinant” therefore indicates that the nucleic acid molecule has been manipulated using genetic engineering, i.e. by human intervention. Recombinant nucleic acid constructs may for example be introduced into a host cell by transformation. Such recombinant nucleic acid constructs may include sequences derived from the same host cell species or from different host cell species, which have been isolated and reintroduced into cells of the host species. Recombinant nucleic acid construct sequences may become integrated into a host cell genome, either as a result of the original transformation of the host cells, or as the result of subsequent recombination and/or repair events.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation or transfection into a host. The cloned gene (inserted sequence) is usually placed under the control of control element or transcriptionally regulatory sequences such as promoter sequences. The placing of a cloned gene under such control sequences is often referred to as being operably linked to control elements or sequences.

A first nucleic acid sequence is “operably-linked” with a second nucleic acid sequence when the first nucleic acid sequence is placed in a functional relationship with the second nucleic acid sequence. For instance, a promoter is operably-linked to a coding sequence if the promoter affects the transcription or expression of the coding sequences. Generally, operably-linked DNA sequences are contiguous and, where necessary to join two protein coding regions, in reading frame. However, since for example enhancers generally function when separated from the promoters by several kilobases and intronic sequences may be of variable lengths, some polynucleotide elements may be operably-linked but not contiguous. “Transcriptional regulatory element” is a generic term that refers to DNA sequences, such as initiation and termination signals, enhancers, and promoters, splicing signals, polyadenylation signals which induce or control transcription of protein coding sequences with which they are operably-linked.

Operably-linked sequences may also include two segments that are transcribed onto the same RNA transcript. Thus, two sequences, such as a promoter and reporter sequence are operably linked if transcription commencing in the promoter will produce an RNA transcript of the reporter sequence. In order to be “operably-linked” it is not necessary that two sequences be immediately adjacent to one another.

Expression control sequences will vary depending on whether the vector is designed to express the operably-linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e. g. SDS gel electrophoresis, gel filtration, centrifugation, ion exchange chromatography, etc.). In addition, the protein of interest can be purified via affinity chromatography using polyclonal or monoclonal antibodies or a specific ligand. The purified protein can be used for therapeutic applications. Prokaryotically expressed eukaryotic proteins are often not glycosylated.

The DNA (or RNA) construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is preferably bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with SI nuclease), as well as protein binding domains (consensus sequences) responsible for the binding of RNA polymerase. Eukaryotic promoters will often, but not always, contain “TATA” boxes and “CCAT” boxes. Prokaryotic promoters contain -10 and -35 consensus sequences, which serve to initiate transcription and the transcript products contain Shine-Dalgarno sequences, which serve as ribosome binding sequences during translation initiation. Non-limiting examples of vectors which can be used in accordance with the present invention include adenoviral vectors, poxviral vectors, VSV-derived vectors and retroviral vectors. Such vectors and others are well-known in the art.

As used herein, the designation “functional derivative” or “functional variant” denotes, in the context of a functional derivative of a sequence whether a nucleic acid or amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention. In an embodiment, the above-mentioned derivative, variant or fragment is an “antigenic derivative, variant or fragment” (e.g., which has the capacity to induce/elicit an immune response against the parental antigen). Thus, the term “variant” refers herein to a protein or nucleic acid molecule which is substantially similar in structure and biological activity to the protein or nucleic acid of the present invention but is not limited to a variant which retains all of the biological activities of the parental protein, for example.

The functional derivatives of the present invention can be synthesized chemically or produced through recombinant DNA technology. All these methods are well known in the art.

As used herein, “chemical derivatives” is meant to cover additional chemical moieties not normally part of the subject matter of the invention. Such moieties could affect the physico-chemical characteristic of the derivative (e.g. solubility, absorption, half-life, decrease of toxicity and the like). Such moieties are exemplified in Remington's Pharmaceutical Sciences (1980). Methods of coupling these chemical-physical moieties to a polypeptide or nucleic acid sequence are well known in the art.

For certainty, the sequences and polypeptides useful to practice the invention include without being limited thereto mutants, homologs, subtypes, alleles and the like. It will be clear to the person of ordinary skill that whether an interaction domain of the present invention, variant, derivative, or fragment thereof retains its function in binding to its partner can be readily determined by using the teachings and assays of the present invention and the general teachings of the art.

The term “mutein” refers herein to a protein having a modified amino acid sequence with respect to the original protein but which is substantially similar in structure and biological activity to the protein of the present invention. A mutein is typically obtained by a mutation or a recombinant DNA procedure.

Compositions within the scope of the present invention should contain the active agent (e.g. peptide, nucleic acid, and molecule) in an amount effective to achieve the desired effect while avoiding adverse side effects. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th ed., Mack ed.)

A polynucleotide of the invention can also be useful as a therapy. There are two major routes, either using a viral or bacterial host as gene delivery vehicle (vector) or administering the gene in a free form, e.g., inserted into a plasmid. Therapeutic or prophylactic efficacy of a polynucleotide of the invention is evaluated as described below.

The invention further provides a composition comprising several polypeptides or derivatives thereof of the invention or vectors (each of them capable of expressing a polypeptide or derivative thereof of the invention).

Treatment may be effected in a single dose or repeated at intervals. The appropriate dosage depends on various parameters understood by skilled artisans such as the route of administration or the condition of the mammal to be treated (weight, age and the like). Vectors available in the art include viral vectors such as adenoviruses and poxviruses as well as bacterial vectors, e.g., Shigella, Salmonella, Vibrio cholerae, Lactobacillus, Bacille Calmette-Guerin (BCG), and Streptococcus.

A “therapeutically effective amount” refers to an amount effective, at dosages and for periods of time necessary, to achieve the above-mentioned desired therapeutic result. A therapeutically effective amount may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting onset or progression of cancer and associated symptoms and disease. A prophylactically effective amount can be determined as described above for the therapeutically effective amount. For any particular subject, specific dosage regimens may be adjusted over time according to the individual need and the professional judgement of the person administering or supervising the administration of the compositions.

Within the scope of the invention are cells (e.g. host cells) transfected or transformed with the nucleic acid or the vector of the invention. Methods for transforming/transfecting host cells with nucleic acids/vectors are well-known in the art and depend on the host system selected as described in Ausubel et al. (Ausubel et al., Current Protocols in Molecular Biology, John Wiley & Sons Inc., 1994). The terms “transformation” and “transfection” refer to techniques for introducing foreign nucleic acid into a host cell, including calcium phosphate or calcium chloride co-precipitation, DEAE-dextran-mediated transfection, lipofection, electroporation, microinjection and viral-mediated transfection. Host cells transfected or transformed with the nucleic acid or vector of the invention can be used as a vaccine (e.g., autologous cell vaccine) in order to induce or increase an immune response against an antigenic epitope or antigen (e.g., STEAP) in a subject. For example, host cells (e.g., APCs, dendritic cells) may be removed from a subject (e.g., a cancer patient), transfected or transformed in accordance with the present invention and re-administered to the patient. Of course, known steps for further cultivating or modifying these cells could be carried-out prior to re-injecting/transplanting them into a subject. For example, cytokines/chemokines or mitogens or molecules could be added to the culture medium.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”, and includes also the term “consisting of”.

The invention will be illustrated by means of non-limiting examples in reference to the following figures.

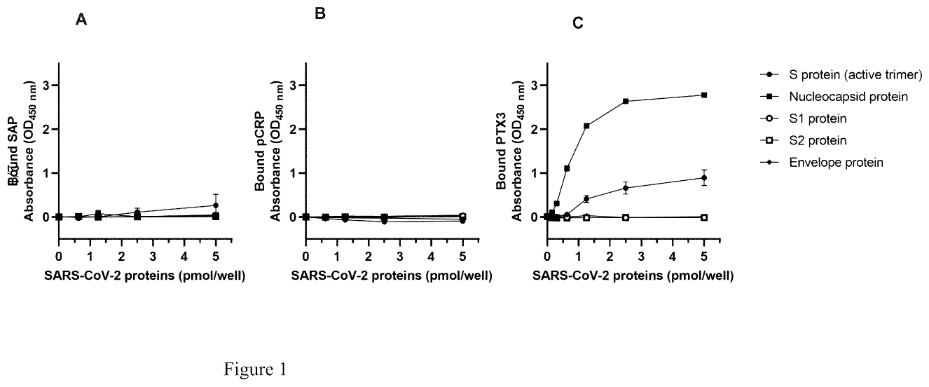

FIG. 1. Interaction between pentraxins and SARS-COV-2 proteins. Recombinant HiS Tag SARS-COV-2 proteins were immobilized on 96-well Nickel coated plates at different concentrations. Fixed concentrations of SAP (A), pentameric CRP (pCRP, B) and PTX3 (C) were incubated over the captured viral proteins. Bound pentraxins were detected by ELISA with specific primary antibodies and data are presented as mean # SEM of two independent experiments performed in triplicate (n=6).

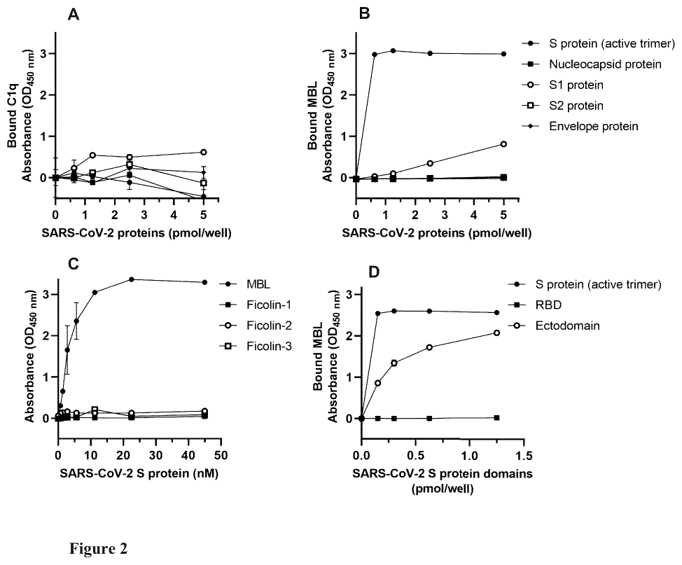

FIG. 2. MBL specifically interacts with trimeric SARS-COV-2 S protein. Recombinant HiS Tag SARS-COV-2 proteins were immobilized on 96-well Nickel coated plates at different concentrations. Fixed concentrations of C1q (A), or MBL (B) were incubated over the captured viral proteins. Bound proteins were detected by ELISA with specific primary antibodies and data are presented as mean±SEM of two independent experiments performed in duplicate (n=4). (C) MBL-, Ficolin-1-, Ficolin-2- and Ficolin-3-coated plates were incubated with various concentrations of biotinylated SARS-COV-2 S protein. Bound S protein was detected by ELISA with HRP-conjugated streptavidin (mean±SEM, n=2). (D) Recombinant trimeric S protein, S-RBD domain and S-Ectodomain were captured on 96-well Nickel coated plates as described above. MBL (2 μg/mL) was then incubated for 1 hour at 37° C. and bound MBL was detected by ELISA (mean±SEM, n=2).

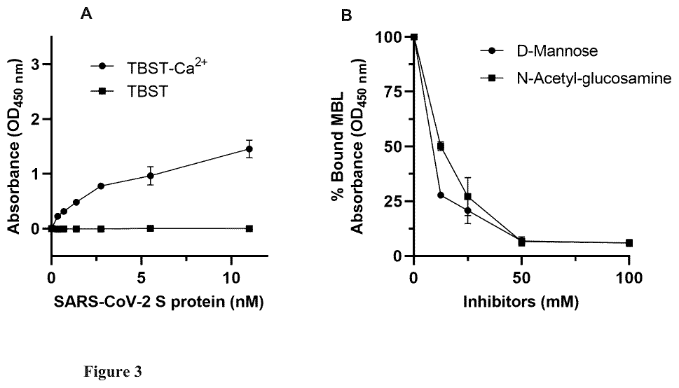

FIG. 3. MBL interacts with SARS-COV-2 S protein through its Carbohydrate Recognition Domain (CRD). (A) MBL-coated plates were incubated with different concentrations of biotinylated SARS-COV-2 diluted in TBST buffer containing or not 2 mM Ca²⁺. Bound S protein was detected by ELISA with HRP-conjugated streptavidin (mean±SEM, n=2). B) Biotinylated SARS-COV-2 S protein was captured on Neutravidin coated plates. MBL (0.625 ng/ml) was then incubated over S protein, alone or in the presence of different concentrations of D-Mannose or N-Acetyl-glucosamine. Data are presented as percentage of bound MBL (mean±SEM of two independent experiments, n=4).

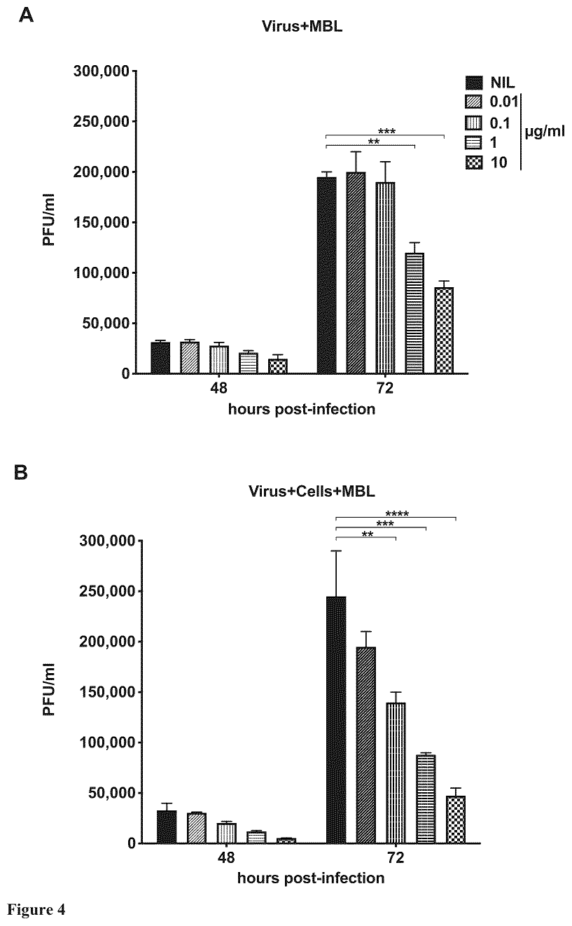

FIG. 4. Inhibition of the infectivity of SARS-COV-2 by MBL. SARS-COV-2 (MOI=0.1) was preincubated in complete medium containing different concentrations of MBL (0.01-10 μg/mL) before incubation with Calu3 cells. After 48 and 72 h, the infectivity of SARS-COV-2 present in cell culture supernatants was determined by a plaque-forming assay in Vero cells. (A). MBL showed a concentration-dependent inhibition of SARS-COV-2 infection of Calu3 cells that was statistically significant at 1 and 10 μg/ml 72 h after infection. (B). When both virus and cells were pre-incubated with the same concentrations of MBL (0.01-10 μg/mL), the antiviral activity increased significantly from 0.1 μg/ml to the top concentration of 10 μg/ml, 72 h post-infection. NIL: no MBL. Mean values of one experiment in duplicate cell culture are shown. ****p value<0.0001, ***p value<0.001, **p value<0.01 as determined by two-way ANOVA with Bonferroni's correction.

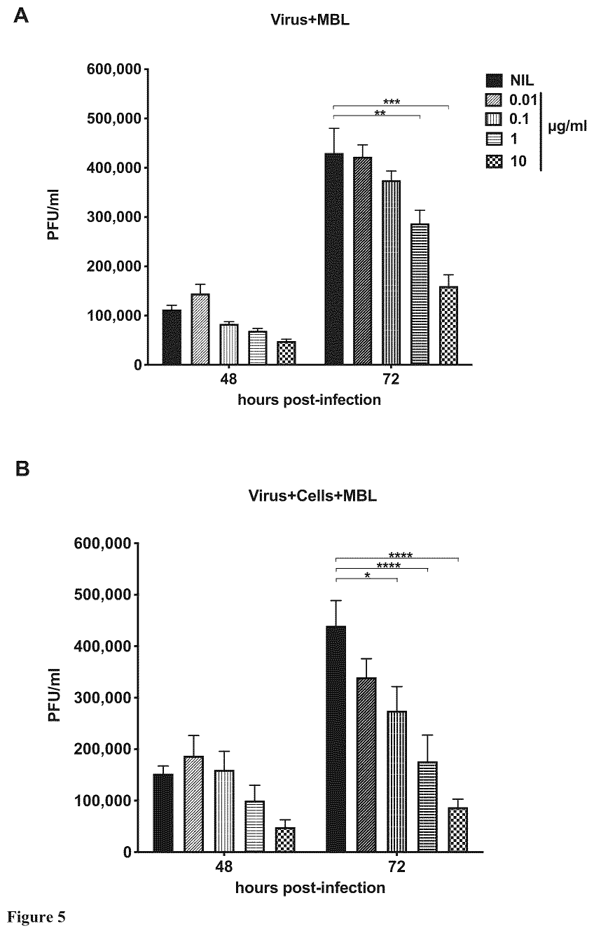

FIG. 5. Inhibition of the infectivity of SARS-COV-2 by MBL. SARS-COV-2 (MOI of 1) was preincubated in complete medium containing different concentrations of MBL (0.01-10 μg/mL) before incubation with Calu3 cells. After 48 and 72 h, the infectivity of SARS-COV-2 present in cell culture supernatants was determined by a plaque-forming assay in Vero cells. (A). MBL showed a concentration-dependent inhibition of SARS-COV-2 infection of Calu3 cells that was statistically significant at 1 and 10 μg/ml 72 hours after infection. (B). When both virus and cells were pre-incubated with the same concentrations of MBL (0.01-10 μg/mL), the antiviral activity increased significantly at 0.1 μg/ml to the top concentration of 10 μg/ml, 72 h after infection. NIL: no MBL. Mean values of two experiments in duplicate are shown. *** indicate a p value<0.0001, * indicates a p value<0.05 as determined by two-way ANOVA with Bonferroni's correction.

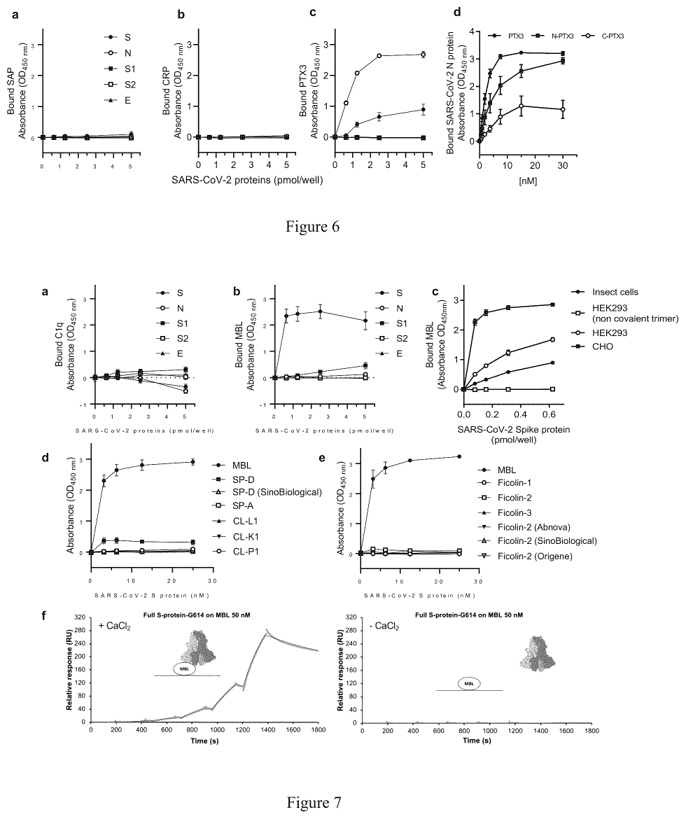

FIG. 6. Interaction between pentraxins and SARS-COV-2 proteins. (a-c) Recombinant HiS Tag SARS-COV-2 proteins (S active trimer, S1, S2, Nucleocapsid (N), Envelope (E); the legend refers to panels a-c) were immobilized on 96-well Nickel coated plates at different concentrations. Fixed concentrations of SAP (a), CRP (b) and PTX3 (c) were incubated over the captured viral proteins. Bound pentraxins were detected by ELISA with specific primary antibodies. (d) Full length PTX3 or its N- or C-terminal domains were captured on 96-well plates. Biotinylated SARS-COV-2 Nucleocapsid protein was incubated at different concentrations. Bound nucleocapsid was detected by ELISA using HRP-conjugated streptavidin. All data are presented as mean±SEM, n=3 independent experiments performed in duplicate.

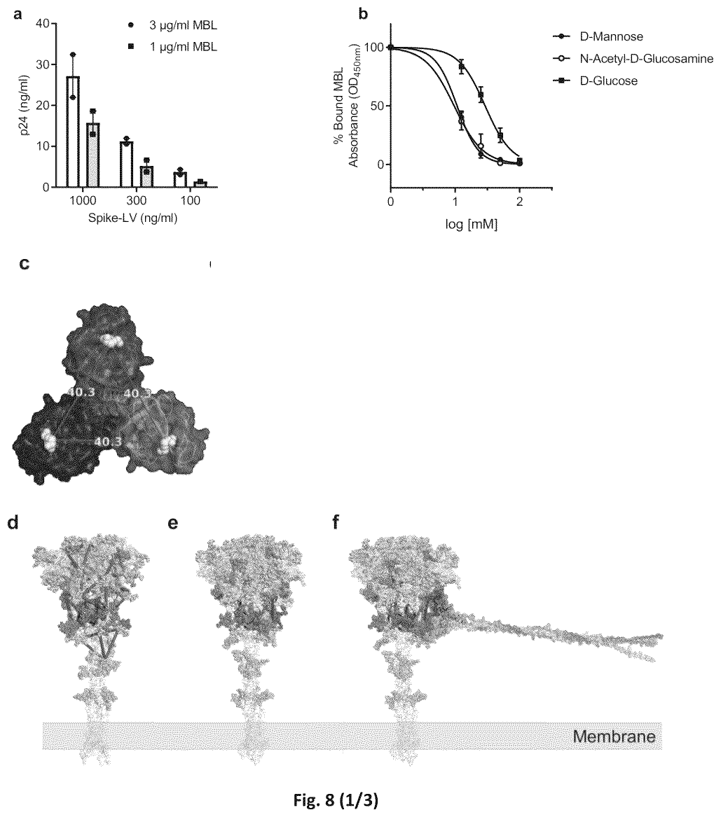

FIG. 7. Interaction of C1q, MBL, ficolins and surfactant proteins with SARS-COV-2 proteins. (a-c) Recombinant HiS Tag SARS-COV-2 proteins (S active trimer, S1, S2, Nucleocapsid (N), Envelope (E); the legend refers to panels a, b) were immobilized on 96-well Nickel coated plates at different concentrations. Fixed concentrations of C1q (a), or MBL (b) were incubated over the captured viral proteins. In (c), recombinant SARS-COV-2 S proteins tested were expressed in different cell types. MBL (2 μg/mL-6.7 nM) was incubated over the captured viral proteins. a-c: Bound proteins were detected by ELISA with specific primary antibodies. a-b: Data are presented as mean±SEM, n=3 independent experiments performed in duplicate. c: mean±SEM, n=2 independent experiments, one performed in duplicate and one in triplicate. (d) MBL-, CL-L1, CL-K1, CL-P1-, SP-D-, SP-A- or (e) Ficolin-1-, Ficolin-2- and Ficolin-3-coated plates were incubated with various concentrations of biotinylated SARS-COV-2 S protein. d-e: Bound S protein was detected by ELISA with HRP-conjugated streptavidin (mean±SEM, n=3 independent experiments in duplicate). (f) SPR shows binding of recombinant full Spike protein trimer to immobilized MBL (K_{D}=34 nM, left). No binding is detected in the absence of CaCl₂ (right).

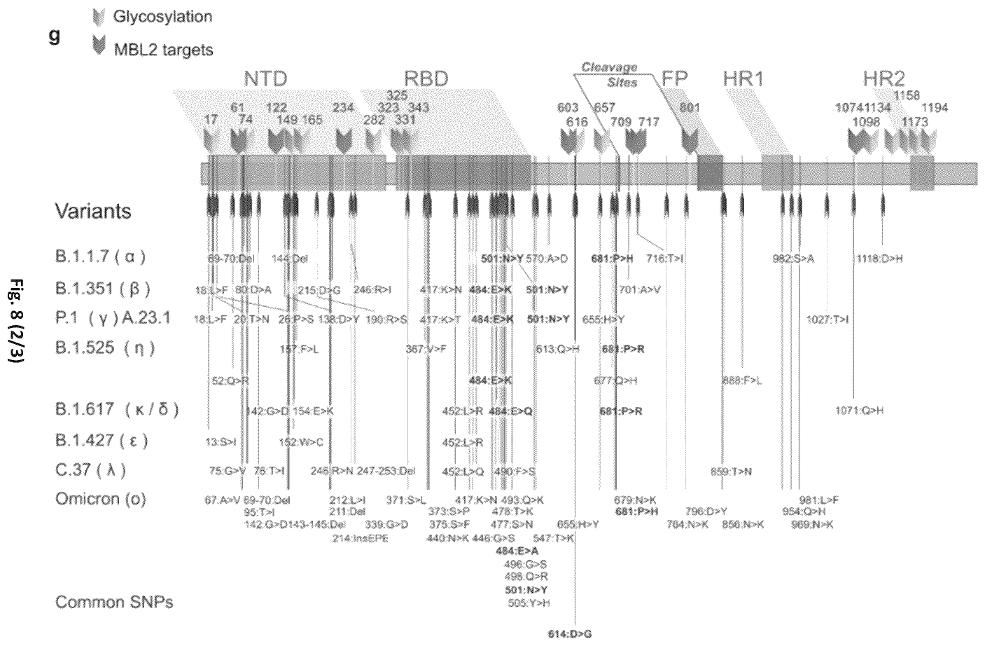

FIG. 8. Interaction of MBL with SARS-COV-2 S protein through its Carbohydrate Recognition Domain (CRD). (a) Binding of MBL to S protein (Spike-LV) or VSV-g-pseudotyped lentivirus (red dotted line). Mean+SEM, n=2 independent experiments performed with 2-6 technical replicates. (b). Binding of MBL to S protein, alone or in the presence of D-mannose or N-acetyl-glucosamine or D-glucose. Mean±SEM, n=4 independent experiments. (c) Trimeric MBL2 model showing the distance (approximately 40 Å) between the binding sites of mannose (white spheres); (d) 14 mannose-binding sites (dark triangles), imposed onto S protein, where S1 region (1-685) is light grey, beginning of S2 region (686-815) is black, and S2′ region is white. (e) Putative binding site of MBL2. Pose with the highest site-specific probability to be glycosylated with oligomannose; (f) Spike-MBL complex. Glycosylation sites are colored according to the oligomannose content: Light grey<60%. Medium grey>80% up until S2′ region. Dark grey>80% in the S2′ region. (g) Schematic representation of glycosylation sites and nucleotide substitutions in the variant strains identified to date. Oligomannose-glycosylated sites are evidenced. SNPs common to all variants are in bold. NTD: N terminal domain; RBD: receptor binding domain; FP: fusion peptide; HR1 and 2: heptad repeat 1 and 2; cleavage sites are reported. (h) Binding of MBL to SARS-COV-2 S protein variants. Mean+SEM, n=2 independent experiments performed in triplicate. (i) MAC (C5b-9) deposition on S protein. Mean±SEM, n=3 of three independent experiments performed in triplicate. Statistical analysis was calculated by Two-way ANOVA, followed by Tukey's multiple comparison test.

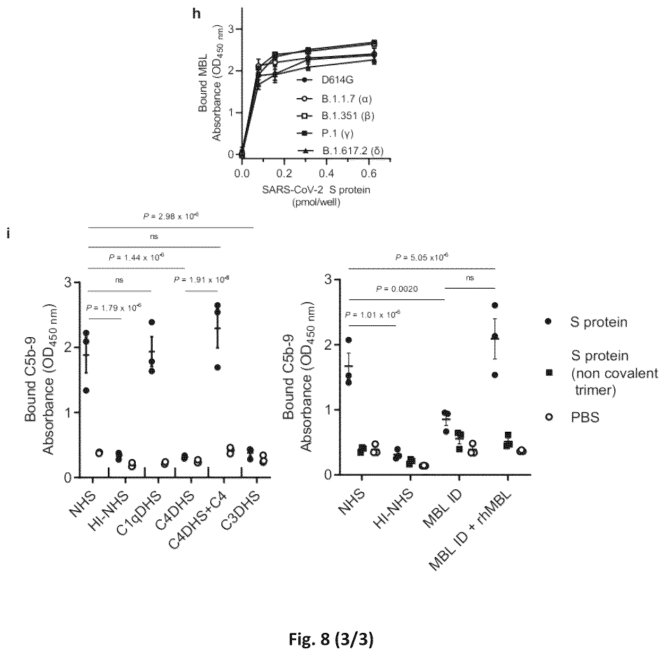

FIG. 9. Inhibition of SARS-COV-2 infection by MBL. (a) Entry of lentiviral particles pseudotyped with SARS-COV-2 Spike protein in 293T cells overexpressing ACE2 in the presence of ten-fold serial dilutions of humoral innate immunity PRMs (from 0.01 to 10 μg/ml). As control, entry of lentiviral particles pseudotyped with the VSV-g glycoprotein was tested in parallel in the presence of MBL. Percentage of control was calculated as ratio of GFP-positive cells in the presence of humoral PRMs and the GFF-positive cells in the absence of humoral PRMs. Data are means+SEM, n=3 independent experiments in triplicates with the curves representing a three-parameter dose response model. (b-f) Inhibition of the infectivity of the D614G (isolate EPI_ISL_413489) (b), B.1.1.7 (α) (c), B.1.351 (β) (d), P.1 (γ) (e), and B.1.617.2 (δ) (f) SARS-COV-2 variants by MBL in Calu-3 cells. SARS-COV-2 (MOI=0.1) and Calu-3 cells were preincubated in complete medium containing different concentrations of MBL (0.01-10 μg/mL-0.034-34 nM; the legend refers to panels b-f) before infection. After 48 and 72 h PI, the infectivity of SARS-COV-2 present in cell culture supernatants was determined by a plaque-forming assay in Vero cells. NIL: no MBL. Mean values, n=3 independent cell culture experiments in duplicate (b-d), or n=2 independent cell culture experiments in duplicate (e-f). (b) ***P value=5.49×10⁻⁴, ****P value=6.38×10⁻⁵; (c) **P value=0.0072, ****P value=5.57×10⁻⁵; (d) **P value=0.0065, ****P value=4.91×10⁻⁵ NIL vs 1, ****P value=7.14×10⁻⁶ NIL vs 10; (e) ***P value=3.40×10⁻⁴, ****P value=6.31×10⁻⁶; (f) ****P value=5.68×10⁻⁵ NIL vs 1, ****P value=4.04×10⁻⁶ NIL vs 10. Statistical analysis was determined by two-way ANOVA, followed by Bonferroni's multiple comparison test.

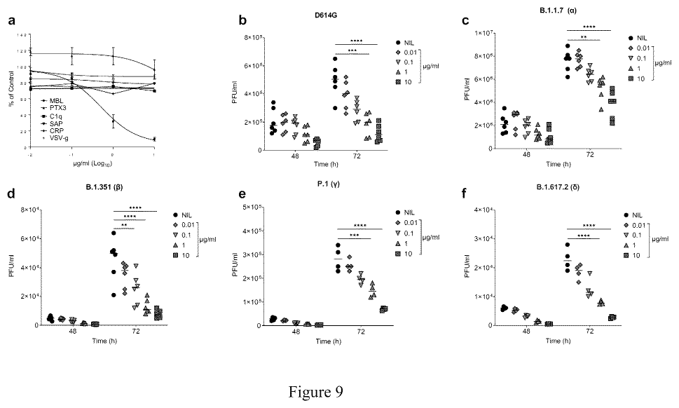

FIG. 10. Inhibition by MBL of SARS-COV-2 infection of primary respiratory cells. (a) SARS-COV-2 production at the HBEC apical surface at 72 h PI, in the presence of 10 or 50 μg/ml (34 or 170 nM) MBL. Mean values, n=3 independent experiments performed in triplicate (2 donors) or single (1 donor) cell cultures are shown. (a) P values were determined by one-way ANOVA with Bonferroni's correction. ****: P value=2.85×10⁻⁵, **: P value=0.0032. (b-e) Colocalization of SARS-COV-2 S protein and MBL in infected HBEC. (b), Confocal analysis of the localization of SARS-COV-2 S protein (light grey), MBL (dark grey) in HBEC cultures infected by SARS-COV-2 in presence or not of MBL (50 μg/ml-170 nM). Left panels, merged images of fluorescence signals; right panels, single signals extracted. Representative MIP images of Z-stacks acquired in tiling modality are shown. N=2 cellular replicates per condition from 1 donor, 4 and 9 Z-stacks. Bar, 30 μm. (c) Left panel: extracted signals of SARS-COV-2 S protein and MBL of b. Bar, 30 μm. Right panel: colocalization rate between SARS-COV-2 S protein and MBL (10 or 50 μg/ml-34 or 170 nM). Each spot corresponds to a single XYZ image presented as MIP. Mean+SEM, basal: n=4 Z-stacks; S: n=4 Z-stacks; S/MBL 10: n=8 Z-stacks; S/MBL 50: n=12 Z-stacks, from 2 cellular replicates. (d) 3D rendering of b right panel, showing a blended reconstruction of the localization between SARS-COV-2 S protein and MBL in HBEC cultures. Left panel, contribution of merged signals. Bar, 30 μm. Middle panel, extracted image of signal of SARS-COV-2 S protein and MBL. Right panel, close-up image that refers to the area dashed in white. 3D rendering of representative Z-stack of panel c. (e) STED analysis of the localization of SARS-COV-2 S protein and MBL in HBEC. N=1 cell culture, 7 STED acquisitions. Left panel, merged signals of SARS-COV-2 S protein and MBL and nucleus; right, single signals extracted. Bar, 3 μm.

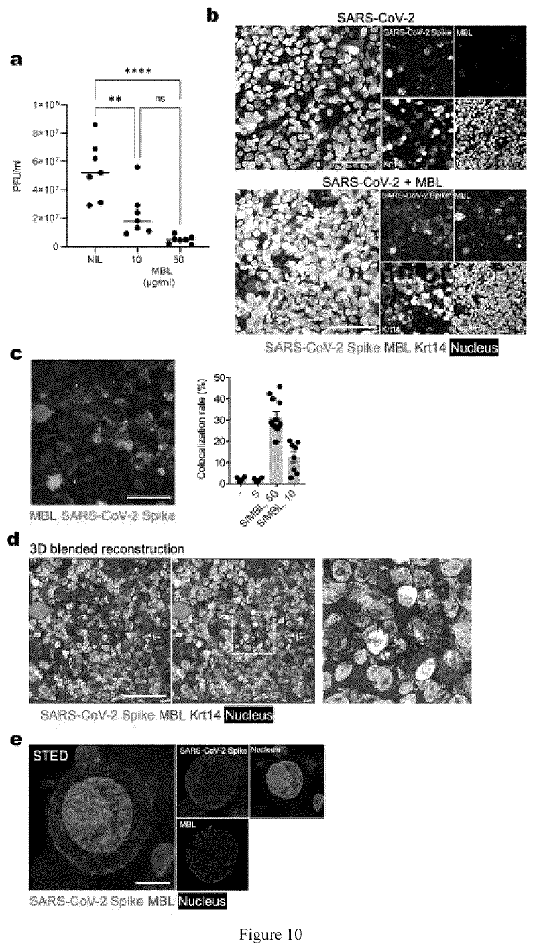

FIG. 11: The MBL2 locus: structure and main association signals with severe COVID-19. (a) A screenshot from the UCSC Genome browser (http://genome.ucsc.edu/; release December 2013, GRCh38/hg38) specifically highlighting the 1-Mb region surrounding the MBL2 gene is shown. The panel reports, in order, the following tracks: i) the ruler with the scale at the genomic level; ii) chromosome 10 nucleotide numbering; iii) the UCSC RefSeq track; iv) COVID-19 risk variants from our study (lollipops show only signals at P<3*10⁻³); v) COVID-19 risk haplotypes, marked by the tagging SNP, from our study (lollipops show all haplotypes reported in Table 4 and Table 3); vi) COVID-19 risk variants from the COVID-19 HGI GWAS Analysis C2 (17,965 cases, 33 studies, Release 4: October 2020); vii) ENCODE data (https://www.encodeproject.org/) for H3K27Ac, H3K4Me1, H3K4Me3 histone modifications marks, all derived from 7 cell lines; viii) the GeneHancer regulatory elements track.

(b) The Manhattan plot of the single-SNP association analysis is reported. The horizontal line represents the suggestive P=5*10⁻⁵ significance level. SNPs showing lowest P value signals are indicated by an arrow. (rs150342746: P=1.86*10−4, OR=3.474, CI=1.808-6.676; rs10824845: P=2.91*10−4, OR=1.762, CI=1.297-2.393; rs11816263: P=3.47*10−4, OR=1.422, CI=1.173-1.725). A logistic regression analysis was used. Bonferroni threshold for significance corresponds to P<1.5*10⁻⁵. (c) MBL plasma concentrations in COVID-19 patients carrying the wild-type allele (A/A) for the rs1800451, rs1800450, rs5030737 SNPs, compared to patients carrying at least one copy of any alternative allele (0). Mean+SEM, n=17 A/0 or 0/0 and n=23 A/A carrying patients. P value was analyzed by two-tailed t-test. (d) MBL plasma concentrations in COVID-19 patients stratified based on the genotypes of the rs 10824845 (wt or heterozygous in our cohort) and the presence of A or 0 alleles, as described for c). Box-Cox transformation was used to normalize data. Mean+SEM, n=10 0/het; n=7 0/wt; n=13 A/het; n=10 A/wt patients. P value was analyzed by ANOVA.

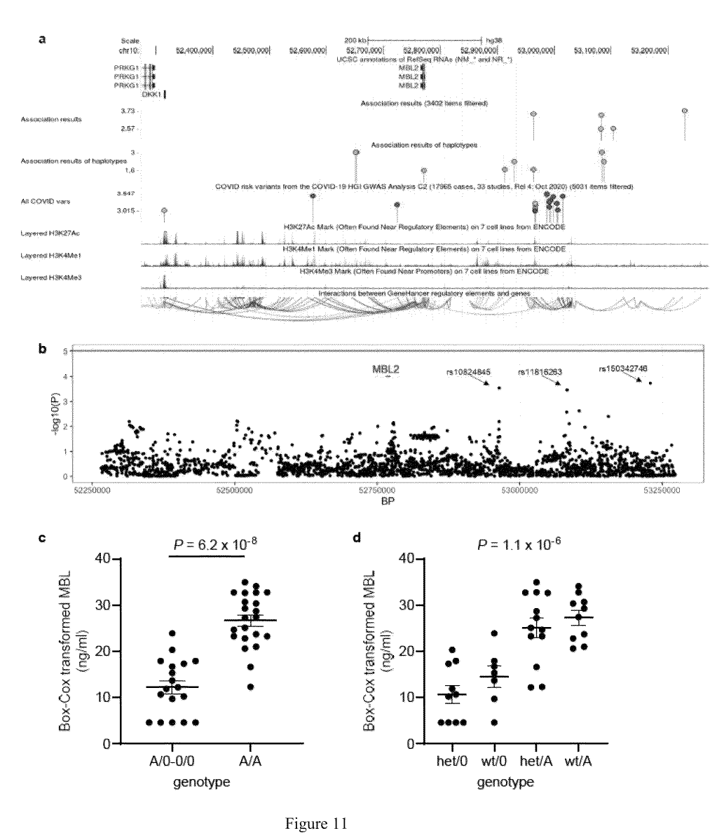

FIG. 12. Interaction between PRM and SARS-COV-2 proteins. a) Binding of PTX3 to captured SARS-COV-2 Nucleocapsid proteins from different companies. b) Binding of PTX3, Ficolin-2 and CL-K1 to SARS-COV-2 N protein-coated plate. c) Binding of MBL, Ficolin-2 and CL-K1 to SARS-COV-2 S protein-coated plate. d) Binding of MBL and CL-L1/CL-K1 heterocomplexes to SARS-COV-2 S protein-coated plate. Data are presented as mean±SEM , n=2 (a, d) or n=3 independent experiments (b, c) performed in duplicate.

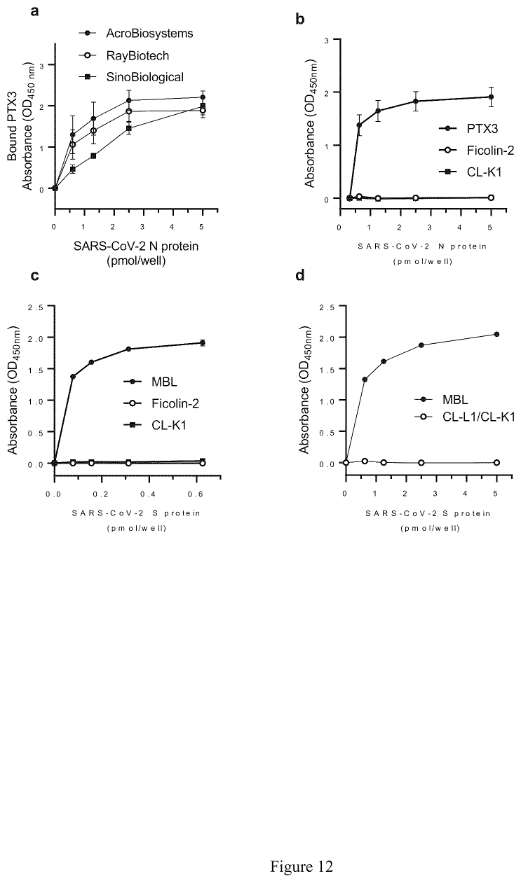

FIG. 13. SPR analysis of RBD and MBL interaction. Interaction between recombinant RBD and immobilized MBL in the presence or absence of calcium, as assessed by SPR analysis.

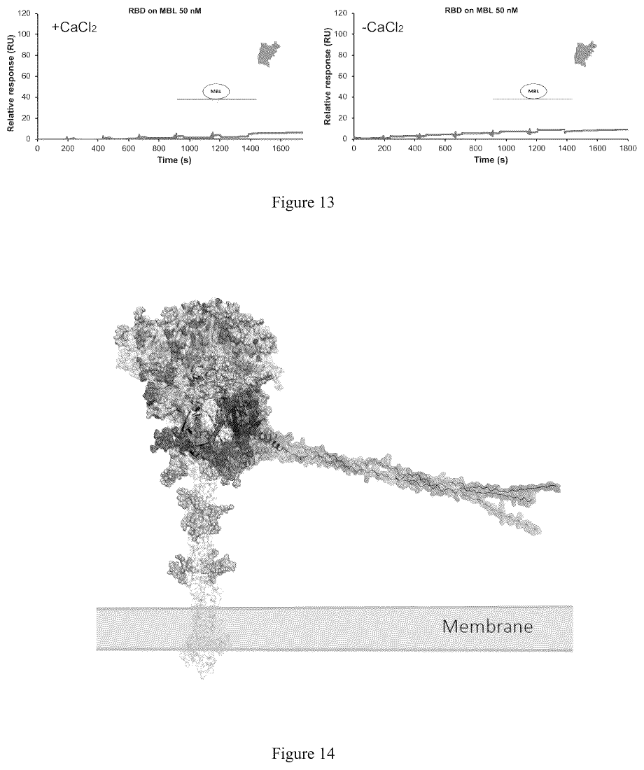

FIG. 14. A model for the Omicron Spike-MBL complex. Glycosylation sites are colored according to the oligomannose content: Gold<60%. Purple>80% up until S2′ region. Blue>80% in the S2′ region. Omicron mutations are in magenta.

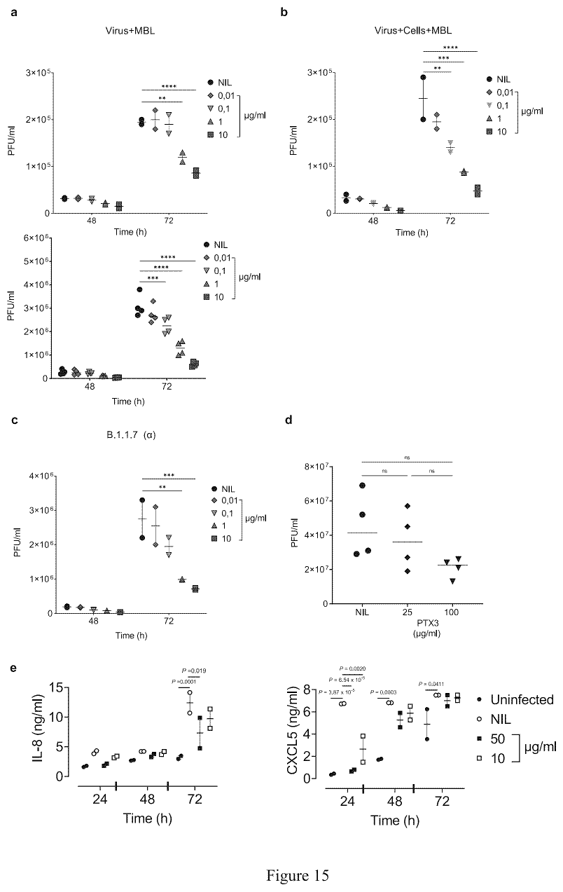

FIG. 15. Inhibition of viral infection and chemokine production by MBL. (a-c) Inhibition of the infectivity of the D614G (a, b) and B.1.1.7 (a) (c) SARS-COV-2 variants by MBL in Calu-3 cells. SARS-COV-2 (a upper panel: MOI=0.1; b: MOI=0.1; c: MOI 0.01; a lower panel, MOI=1) was preincubated in complete medium containing different concentrations of MBL (0.01-10 μg/mL-0.034-34 nM) before incubation with Calu-3 cells (Virus+MBL) (a), or both virus and cells were pre-incubated with the same concentrations of MBL (Virus+Cells+MBL) (b, c). After 48 and 72 h, the infectivity of SARS-COV-2 present in cell culture supernatants was determined by a plaque-forming assay in Vero cells. NIL: no MBL. (d) SARS-COV-2 production at the HBEC apical surface at 72 h PI, in the presence of 25 or 100 μg/ml (75 or 300 nM) PTX3. (e) Chemokine production by SARS-COV-2 infected HBEC in the presence of MBL. Mean values, n=2 (a, b, d) or n=1 (c, e) independent experiments in duplicate cell culture are shown. (a, upper panel) **P=0.0011, ****P<0.001; (a, bottom panel) ***P=1.63×10⁻⁴, ****P<0.001; (b) **P=0.0027, ***P=1.35×10⁻⁴, ****P<0.001; (c) **P=0.0024, ***P=8.32×10⁻⁴ Statistical analysis was determined by two-way (a, b, c, e) or one-way (d) ANOVA, followed by Bonferroni's multiple comparison test.

Sequences

Homo sapiens mannose binding lectin 2 (MBL2), RefSeqGene (LRG_154) on
chromosome 10
NCBI Reference Sequence: NG_008196.1
GenBank Graphics
>NG_008196.1 Homo sapiens mannose binding lectin 2 (MBL2), RefSeqGene (LRG_154)
on chromosome 10
ACACCCTCTGACCACCCACCCCATGGCTATAGGGGCCTAAGGTAGTCCTTGCCAGATGCAGTGCGCCTGT
GTATTTGTGTTGGTTAAAAAGAAGGCCTGTGACATTTACACATTCTCAGAGAGTTCATGAGCAAAAACAA
GCAAAGAAAAAATAGCAGGACTGGAACACAGGACAGTACAGGATGAAAAGTATGGGAAGGTCAGGCCTTA
GGGTTACAAAGGACACTGCTGACAACAAGAAAGCAGCCTACCGCTGTCAGCTGTCTCTCACAGCCTGTCT
CTCATGGCCAGACCATGGTATTCCCTGCTGACCCTAAACACTTCCACAGAATACCGGTGTACAACAATAT
TACTCTGTTGTGAGATCCCAAATCATTCCAAAATCATGTCTTCATATAGACAGAAAAGGTTGCTCTGTGA
ACCACAAAAACGAAAAACATGCCCCTCTCCTGCTTAATATAAACATCTGCCACTCTTTTACAAAATACAG
CTTCGCCTTGCTCTGTTCTACTTATCTCCTAAATAAAAGTTAAGATGCACAATCACAGAATTGCCCAGCT
TACGGACAGCACCCTATCCAGAACAGACTCCTGCTTCTTAAACCCCTTCCCCCAAAACCAACTAAAACAA
AACAAGTCTTATAGAAATCCCCTCCCGACACCCTGTTACTGAGAAATCCTAGGGTACACCATTGTGCTCA
ATATCCCTGTTGGAACAAACAGTTAAAAGAAACTAAAAATAATAAATCCATTTTATCTGACTATAAGCAT
ATTCCTGATGGTCCTCACTTGGAGAGCAATAACATTCTCATTGCTATTGTCCTACTCACAGCAGCTCTGT
GGAAAAGGGAATGAGGTTCAAAATCTGATTTGAAAGATGTAAAACTGAGGTTCGGCCAGAATATGGGGTC
TATCTAAGCTCATATTCATGAGACAAAACCAAGCCTTCTGGTTTCCAATCTCAAACCATGCCTCTCACAT
GGAGCCACCCAAGAACACTGCTTCCCAAGCTGAGAAGACTCTTGAACAGTCACCCTGGAGGGAATGAAGG
CCTTTCTTAAGCAGGCTGTCATGGTGAGCCACGTGCCATGCACTCTGAATGTGGACCAGTTAAGGACACA
CCACTCCACAGCCAAGGCAGCGGTGCCCTCTTTCCCAGACGAGGAAACTGAGGACATGAAGGCAGTGGAA
CTTGAATAAGGTCACACTGTTGGGAAGTGGGAGAGCTGGGATTTCACCCTAGTCTACAAAAAAGGGGATG
TAGGGCACCAGGCAGTCCTCCTGCAGGTCAGAGAGGCAGTTTTCTCTGCAGCCTCTTGGGGTGAGGTGGA
TTTGTTGTGGCGCTGCCAGAATGGAAGCAGTGTTAGGAAAGTGGAAAGTTTTTTTTTTTTTTAGACGGAG
AGGCTCTGTCGCCCAGGCTGGAGCGCAGTGGTGCAATCTCAGCTCACTGCAAGCTCCGCCTCCCGGGCTC
ACTCCATTCTCCTGCCTCAGCCTCCTGGGTAGCTGGGATTACAGGCACCCGCCACCACGCCCGGCTAGTT
TGTTGTTTTTTTTTTTTTTGTATTTTTTAGTAGAGATGGGGTTTCACTGTGTTAGCCAGGATGGTCTCAA
TCTCCTGACCTCATGATCCGCCCGCCTCGGCCTCCCAAGGTGCTGGGATTACAGGAGTGAGCCACCACGC
CTGGCTGGGAAGGGTTTTAAAGTCCAGAACAAATGCACACAGTGGCCTTATCTATGGGGTGGAGTGCAGA
GTGGGCATTCGTGTGGAGAGAGACATTCAGGCTGCGGCATGTGGAGAGTGGCCTCTCCTTTTGGGAAAGG
CCTTCTGGAGAGCAGGATGTAACTGGGTGACACTCCAATCTCCGGATCAATTGCTAGATTGACTTTCCCC
CAGATACCCACACCCTCTGAACCCAGTTCTAGGAAATTTCAGGGTCTGGAGAGCAGTTTCCTGACCTCTG
GACTAGGTAAACAATGAAAGCAAGCACTGTGATTAAAGCCAGCACCGCCAGTGACCTGGAGTCAACTACC
TCACCTCACCTTCTGCCACTGGAGCAGGAGCAGGGGGAAGCTCTGGCTGCTAAAGAATAAGAAGGGAAAC
AAGTCTTCTCAGGCCCTGTCTTCTTTAGGCCCTGGATCTCCTGATATCCACTGATGGCTTTAGGCATGTG
GCTCTGTACCATCCTGGACATTTTTCCAGGCACAGCAGACAGGTCAGCAGGAAGTAAGGAAGCCAGTCCC
TCCACAAGCCAAGAAGACTTCAATTGGAAGGGACCTGGAAATGCATCTTACTCACATTTCTTTTGGTGTT
TGGAGGCCTCCATAGTACCCCCACCACATGGCCCCGCAGCCTCTGAACTTCTCAGGATGGGCTCCCCAAC
ACCCCAAGCTACATGGCATTTTTCTGAAAGCCATATCAGTAATGTAGTTGAGAATCAAATGAACTATGTT
CGTAAAAGTATTCTTCCATCCCTTCTAGATTGTCCCCATTAACCACAGCAAGATGGTTCAAGCCATTAAC
ACACACGATACCCATGATCTTATAGTGCCAAGTGCTGTTCTTTTGCTTCTTATCTCAGTTGAGTGGCTCC
TAGATAGTCAAGTCATAAATGTGTGTGCCCATGAAAACTTCTTATTATCACTCACTATTCTAGCTAATTC
TCAAGTGCAATCAATACCTCCTGTTTCTCAAGTGTGCCTACCTTCTTAATCTAGCTGCCATTACCCTGAC
CCAGGCCACTGTCTTCTGCTGCTTAGATCATGGCAAGCACTTCCTTTTAGATCTGCTTTTCTCCAGTCCT
GTTCCCCTCAACATCATAGCCAGAACAAGCCTTGCAAAATTCAAATGTGATCACCTCTCCACTACTGCAG
TCCCTCTAGTGCCCCCTGGAAGGAAACCCAAAGTCTTTAACACAGTTTACAATTCTTGTTATGAGGATGA
AATTTGAAGACATTGTGCTAAGTGAAATAAGCCAGATACAAGGGGGCTATTGTATGATTTCACATATATG
AGGTACCTAGGATAGATGAATTCAGAGAGACAGAAAGTAAAACAGAAGTTACCAGTGGCTGTGGGTAGGG
GTAAATGGAGAATTCTAGTTTAATGAGTTGAGAGTTTCAGTTTGGGATGATGAAAATCATCCCATGATGT
GGTGATGATAGCACAACCGTGTGAATGTACTTAATGCTACTGAGTTGTGTGCTTAATAATAATGGTCAAA
ATGGTAAATTTTATGTCTATTTTACCACAATGAAAAACTGGAAAAGTAAAGTCAATGACTATAATATCAT
AAAGTGTAAGTAACATGAGAGAAAATAATTCAGGCTAAGAGGATAGAGACTGCTAGGTAGGACTATTTTA
GAATAAGTGATCAAGGTACTGATTTCTCTGAAATTGGAATATTTGAATAGAAGAGAAAGAGCTAGCTGGC
GATGCCCGTAAGGGGTCCAGGCATGGAGACATCAAATGAAGAGTCTGAGGCAGGAATGGCCTTAATGCAT
TAATGGCAGCAAGAGGTCTATGTGGTTTCATGGATGGGTGTGTGCGTGCATGCACGTGTCTGTGTGTATA
GTTGGTGTGCATGAGCATGTGTATGCATTTGTGAGTATCTTTGTTTTTCTGTTGGAAACAAGGATGTCAA
CAGGACTAACTAGGTGAACCTAGCTGTGGTAGTTCAGCAATGAGATTTGCCAAAATGGATGGCAATTGAA
AGAGGAATTGTGCTGTATTAAAGCAGACAGCTGCCGAGCACTGTATTGGAGGCTCCTAAATTTCTCATAG
TTCTGAGAAGAGCTGTTTATTAATCTGGAGTTGAAGGCCAGGCCCAGGATATCCTTGTTCACAAGGTTGA
TTGGGCCCTACATTGCTGAGCCCAGCCTCCTCCCTCACTCTGAGGCATCTGCCAGAGCCCCAGGCTAGAG
GGCCAGCGTCCTTGTCACTGAGTCCCTGCTCTGCAGAAACACCAGTAAGTCATTTCTGGTGAATAAATCT
GGGTTTCCGGATCAAGGGTTTGGGGAGCTGACATGACCCTGGAGGCATGGAGGAGGCTTTCTAGGGGTGG
GGGGAGTGGGATGGAAAGCAGAAATCAGGTTGAGGATGTCTCTTTTTCTGCTTGTGCTGGAGCATCTGTG
ACTTTCCTCCATTATCTGTTCATCTGCAGTGGAGACTGTCTTTGTTTTCAAAGGGAAACTTGGAGGCTTA
GACCTATGGGGCTAGGCTGCTGAGGTTTCTTAGGGGGCAATAGCTGGAAGAAAGCTCTGAAGAACAAATG
AAAGGTTAATACTGAGAAATGGGAGGAGGATTCAAGGCAAGTTTTCTAATTGCCAGTGGTTTTTGACTCA
CAGAACATGGGGAATTCCTGCCAGAAAGTAGAGAGGTATTTAGCACTCTGCCAGGGCCAACGTAGTAAGA
AATTTCCAGAGAAAATGCTTACCCAGGCAAGCCTGTCTAAAACACCAAGGGGAAGCAAACTCCAGTTAAT
TCTGGGCTGGGTTGGTGACTAAGGTTGAGGTTGATCTGAGGTTGAGACCTTCCTCTTTGGATCACCAGCT
TTCAGCTCAGGGCCTGCCAATGAGTAAATGATAGTTAACAGGTCCTGGAGGGGAATCAGCTGCCCAGATA
CAAAGATGGGATTCAGGTGGCAGATGGACCCGAAGAGGACATGGAGAGAAAGAGGAAGCTCCTACAGACA
CCTGGGTTTCCACTCATTCTCATTCCCTAAGCTAACAGGCATAAGCCAGCTGGCAATGCACGGTCCCATT
TGTTCTCACTGCCACCGAAAGCATGTTTATAGTCTTCCAGCAGCAACGCCAGGTGTCTAGGCACAGATGA
ACCCCTCCTTAGGATCCCCACTGCTCATCATAGTGCCTACCTTTGTTAAAGTACTAGTCACGCAGTGTCA
CAAGGAATGTTTACTTTTCCAAATCCCCAGCTAGAGGCCAGGGATGGGTCATCTATTTCTATATAGCCTG
CACCCAGATTGTAGGACAGAGGGCATGCTCGGTAAATATGTGTTCATTAACTGAGATTAACCTTCCCTGA
GTTTTCTCACACCAAGGTGAGGACCATGTCCCTGTTTCCATCACTCCCTCTCCTTCTCCTGAGTATGGTG
GCAGCGTCTTACTCAGAAACTGTGACCTGTGAGGATGCCCAAAAGACCTGCCCTGCAGTGATTGCCTGTA
GCTCTCCAGGCATCAACGGCTTCCCAGGCAAAGATGGGCGTGATGGCACCAAGGGAGAAAAGGGGGAACC
AGGTACGTGTTGGGCTGTTCTGTCTCTGCAATTCTTTACCTTCCAGAGGAAACTGCCTGGGGATATGAGG
AGACTGATGTCCTATTTGAGTATATTTTTCTCAACTATACTGTAACTCAAAACAGAGATTCAGCTCTCAT
TCTCTGGCATCTCAGAATTCCACACAGCAGTTTGTGACTAATAGTTGTCTTGCCAGCCCAGGAAAGTGGC
CCACAGGTCAGGCCATCCCGTGGGACACAGGATGAATTTTTCTTCTCTGGGTCATTGTCATGTCAGACCC
CTATTCACTTCAGTAGGGATGGCACCAGGTTCAAGAGGCCAAAGAAGAGATGGAGTCAGCAAACAAACAT
AGGTTTTACTGGGGGAATCTGTTTACAGGGAGATCCAGCAGCAGTGGGCTGGACAGGAGAACAACAACTA
CTGGTAAAAACAAATGCAGTTAATTTTCACTTTGCACCCTCCCTGCAGCAACCTCCACGTGGCAACTTTA
TTTCTTAAGTTATTGCTCTCAGGTGCCCACCATACAGTTATTGAGAGCAGTGCTCAGAAAGGTCAGTCCT
GGGTCAAGGTCTCCTTTCTCCTGAGAAGGGATTGGGCATCAAACTCTTGAAGAGAGAGAGCAAGAACATA
GATATTAAGTCACATTTCCTTTGTCTTCCAACAGGCCAAGGGCTCAGAGGCTTACAGGGCCCCCCTGGAA
AGTTGGGGCCTCCAGGAAATCCAGGGCCTTCTGGGTCACCAGGACCAAAGGGCCAAAAAGGAGACCCTGG
AAAAAGTCCGGGTAAGGACCCCAGCAAGGTCTGAGCTGACTTCACCCAGGGTTCTGAGACCTTGAGTATC
TGGTAAGAGGTGCCCCTTCTCCTGTTCCTTCAAAGGAAGATACCCAAATTTGCTTTCTGACCCAGTGCCC
TCAGCCCTCTCTTCATTCTTAGAGCCTTAGGGAATTCCTATAAGTGCCTGAGTACAGACTTACAAACTTT
TCAAGACAACTCCCTCATCAGGCACTCCCTGTAAGAGCTGAGCCTGGATTCTCCATCAGCTGAAACAGTC
TAATGTGAGGCCCTGGTCCTAGAGGGAGAAAACAGGCCATCTAAGTGTGGGCTTATTATACCTATTTTTA
CAAAAGCATGTAACCATGGTATGTCATATTTTACTTCATAAGAAACCAAGCCAAAGCTTAATAGCTTAAA
AACATAGCCGTCAATTTAAATCACAAGTCTGTGGGTCGGCCATGTGATTTCTCTGCTGGTTTAAGCTGGG
CTTACCCACATGTCTGTGGTCAACTGATGGCCAATAGTCTCACTCATGTGTCTGGCTGTTGTCTGAATGT
TAGCTGGAGTGATGAGGGTAATTGAACCACATCTCTCATCATCCAACAGGGCTTGTTCACATAGCAGTGA
TTACAGGATTCCCAAGAGTAGAAATAAGATAGGCTTTAGGCTTCTTGAAGACTCAGAGCACATTATCATT
TCTGTCAGACTGTATTGGTCAAAACAAGTCACAAGGCCAGCCCAGATTCAAGGAGTGGGGTAATAGACTC
ATGTCTTAATGCGAGGAGCTGTACCCTATTGAGGCCATTTTTGCCATCTACTACAACAAGTGATTTGCTC
ATGCCCTCCCCACTTGGATAATGGGCAACTCACAAAAATCTTGGTTTCCTGACTAACAGAACAAACCTGG
AAAAAACAAAAACAAAACAAAAGACATCAAAAGCAAAACCTCTCCAAAGACAAAAACTCTGCAATCTTTT
TCGTCACTCTTCACCAGGTCATAATAGAGTAACGCCTCAAGAGATTTCTTTTTAGTTTTGATAGAACAAT
TTATTTGAAAAAAAAATCTGTGAAGAACCTGGCATATGGATTGCTAGGTAATCAGGGAGTATATGTGTGC
ATGTGATATGTATGCGTACTTTGGGATACAGTAAACAGACAAAGAGGGACATTTGGGGTTGGATGGAAAT
AGCGATTTTTCATTCTTTGGGCTTTTCAGTATTTTTTATTTTCCTTCAAAGTGTAAACATCAATGTAAAA
TAAGCTTTTTTTTTCAGTTTGAAAAAGATACTCCACTCCCTTGTATATGCACAGTTCTGAGGCTCCTTCT
CAACAATGCTTCTTTACTTTGTTCTAATTTTCTAGATGGTGATAGTAGCCTGGCTGCCTCAGAAAGAAAA
GCTCTGCAAACAGAAATGGCACGTATCAAAAAGTGTAAGCTTTTTCTCTTACTCTCCAGGCAGCTTGAAG
TTTGGGAAAAATAGAATGCAACAAATATTTGTTGAATGCATATAATTTTCTGTACCCTGCTAGGCATTTC
TCATATTCTTACCTCATGAAATTCTCACAACATTTTGGTAGAAATGGAGGCAAAGGGAAGTTAAATTACT
TGTTCAAATGCACAGAGCTAATAAATGGCAGGGGTGGTTTATAGATGGAAGTCAGTCTGACTCGAGAGAC
CCTAATCCTTTACCGTCTGATATTGCTCACTGAAAATGGGACTTATATCCCTTTGTTGCACTGGTATTGA
GACCTGGCCGTGGGGTCTAACCTGCCTGGGGCAAATATTTTCAGACATTTTTTGTTTGGTCTCAAGTTAA
CAATTTAGAATTCAGAAGTCCAAATTATATGTCTTTTAGAATTCTGATCTGAAAGCACAGAGAGGCCTTT
GTACCAGTCTGTCTGTTCACATTTGGGTTGCCATATTTAACAAATAAAAAGACAAAGCACCAGTTAAATT
GGATTTCAGCTGAAAAACATTTTTAGTATAAGTGTGTCTGAAAAATTATATGGGAGATATACTAAAATAT
TCATTGTTTGTCTGAAATTCAAATTTAACTGGACATACTATATTTTATCCGGCAACTGTACTCTAGAAGA
CTTTTTCTTGAGAAATACCTTGAGTTGGGCTTAAGGATGAGTCAGTTTCACCCACTTTTTCACATTTTAG
GGCTCACCTTCTCTCTGGGCAAACAAGTTGGGAACAAGTTCTTCCTGACCAATGGTGAAATAATGACCTT
TGAAAAAGTGAAGGCCTTGTGTGTCAAGTTCCAGGCCTCTGTGGCCACCCCCAGGAATGCTGCAGAGAAT
GGAGCCATTCAGAATCTCATCAAGGAGGAAGCCTTCCTGGGCATCACTGATGAGAAGACAGAAGGGCAGT
TTGTGGATCTGACAGGAAATAGACTGACCTACACAAACTGGAACGAGGGTGAACCCAACAATGCTGGTTC
TGATGAAGATTGTGTATTGCTACTGAAAAATGGCCAGTGGAATGACGTCCCCTGCTCCACCTCCCATCTG
GCCGTCTGTGAGTTCCCTATCTGAAGGGTCATATCACTCAGGCCCTCCTTGTCTTTTTACTGCAACCCAC
AGGCCCACAGTATGCTTGAAAAGATAAATTATATCAATTTCCTCATATCCAGTATTGTTCCTTTTGTGGG
CAATCACTAAAAATGATCACTAACAGCACCAACAAAGCAATAATAGTAGTAGTAGTAGTTAGCAGCAGCA
GTAGTAGTCATGCTAATTATATAATATTTTTAATATATACTATGAGGCCCTATCTTTTGCATCCTACATT
AATTATCTAGTTTAATTAATCTGTAATGCTTTCGATAGTGTTAACTTGCTGCAGTATGAAAATAAGACGG
ATTTATTTTTCCATTTACAACAAACACCTGTGCTCTGTTGAGCCTTCCTTTCTGTTTGGGTAGAGGGCTC
CCCTAATGACATCACCACAGTTTAATACCACAGCTTTTTACCAAGTTTCAGGTATTAAGAAAATCTATTT
TGTAACTTTCTCTATGAACTCTGTTTTCTTTCTAATGAGATATTAAACCATGTAAAGAACATAAATAACA
AATCTCAAGCAAACAGCTTCACAAATTCTCACACACATACATACCTATATACTCACTTTCTAGATTAAGA
TATGGGACATTTTTGACTCCCTAGAAGCCCCGTTATAACTCCTCCTAGTACTAACTCCTAGGAAAATACT
ATTCTGACCTCCATGACTGCACAGTAATTTCGTCTGTTTATAAACATTGTATAGTTGGAATCATATTGTG
TGTAATGTTGTATGTCTTGTTTACTCAGAATTAAGTCTGTGAGATTCATTCATGTCATGTGTACAAAAGT
TTCATCCTTTTCATTGCCATGTAGGGTTCCCTTATATTAATATTCCTCAGTTCATCCATTCTATTGTTAA
TAGGCACTTAAGTGGCTTCCAATTTTTGGCCATGAGGAAGAGAACCCACGAACATTCCTGGACTTGTCTT
TTGGTGGACATGGTGCACTAATTTCACTACCTATCCAGGAGTGGAACTGGTAGAGGATGAGGAAAGCATG
TATTCAGCTTTAGTAGATATTACCAGTTTTCCTAAGTGATTGTATGAATTTATGCTCCTACCGGCAATGT
GTGGCAGTCCTAGATGCTCTATGTGCTTGTAAAAAGTCAATGTTTTCAGTTCTCTTGATTTTCATTATTC
CTGTGGATGTAAAGTGATATTTCCCCATGGTTTTAATCTGTATTTCCCCAACATGTAATAAGGTTGAACA
CTTTTTTATATGCTTATTGGGCACTTGGGTATCTTCTTTTGTGAAGTACCCGTTCACATTTTTGTATTTT
GTTTAAATTAGTTAGCCAATATTTTTCTTACTGATTTTTAAGTTATTTTTACATTCTGAATATGTCCTTT
TTAATGTGTATTACAAATATTTTGCTAGTTTTTGACTTGCTCCTAATGTTGAATTTTGATGAACAAAATT
TCCTAATTTTGAGAAAGTCTTATTTATTCATATTTTCTTTCAAAATTAGTGCTTTTTGTGTCATGTTTAA
GAAATTTTTGCCCATCCCAAAATCATAAGATATTTTTCATGATTTTGAAACCATGAAGAGATTTTTCATG
ATTTTGAAATCATGAAGATATTTTTCCATTTTTTTCTAATAGTTTTATTAATAAACATTCTATCTATTCC
TGGTAGAATAGATATCCACTTGAGACAGCACTATGTAGGAAAGACCATTTTTCCTCCACTGAACTAGGGT
GGTGCATTTTTGTAAGTTAGGTAACTGTATGTGTGTGTGTCTGTTTCTGGGCTGTCTATTCTAGTCTATT
TGTTGATGCTTGTGTCAAACAGTACACTATCTTAATTATTGTACATTTATAGTTGTAACTATAGTCCAGC
TTTGTTCTTCTTAAAGTCAAGATTTCCATATAAATATTAGAAACAGCTTCTCAATTTCTACAAAATCCTG
ATGAGGTTTCTACTGGGACCACATTGAGTCTATCAATCAACTTATGCAGAACTGGCAACTTACTACTGAA
TCTCTAATCAATGTTCATCATGTATCGCTTCATGTAACTAGAATTTCTTTAACTTAATTGCTATGTTTTG
ACATTTTTAGTTTAAAAACCTTGTATATCTTGTTTTGGTGGTTTTAGTGATTTTAATAATATATTTTAAA
TATTTTTTCTTTTCTATTGTTGTACACAGAAATACAGTTAAGTTTTGTGTGTAGTCTTACGATGTTTAGT
AAACTCAATAAGTTTATTTCTTAAATCTAGTAATTTGTAGATTCCTCTGGATTTTGTATATGCATAGTCA
TGTAAGCTGAAAATATGGCAATACTTGCTTCTTCCCAATTGCTTTACCTTTTTTCTTACCTTATTGCACT
GGTTAGCAACCCCAATACAGAGACCACCAGATCAGGTATAGACTCCTGAAAGACAATATAATGAAGTGCT
CCAGTCAGGCCTATCTAAACTGGATTCACAGCTCTGTCACTTAATTGCTACATGATCTAGAGCCAGTTAC
TTTGTGTTTCAGCCATGTATTTGCAGCTGAGAGAAAATAATCATTCTTATTTCATGAAAATTGTGGGGAT
GATGAAATAAGTTAACACCTTTAAAGTGTGTAGTAAAGTATCAGGATACTATATTTTAGGTCTTAATACA
CACAGTTATGCCGCTAGATACATGCTTTTTAATGAGATAATGTGATATTATACATAACACATATCGATTT
TTAAAAATTAAATCAACCTTGCTTTGATGGAATAAACTCCATTTAGTCACATTTTATCTTGTGTATGTAT
TATTGGAGTGGACTTGTTAATAGTTTGTTTAGAACGTTTGCACCTATGTTCATGAGAGATACTAGCTTTT
TTTCCCCTCCTTAATGCACTTGCTATGGTTTGGGGTCAATGTTATGTAAGCCTCATTAAAATGAGCTGTG
ATGTGCTCTCATGCTCCTTATTATCTAAAATAATTTGTGTAAAATTATACTTTAATGTTAGATAAATTCA
CCAGTGAAGCCATCTTGGAATGGTGTTTTCTGTTGGGAAATGATTTTAATTTCAGATTTAATTTCCTCAT
GATCTAAACCTATTCATGTTTTCTGTCTTTTGTTCGGTTTCAGTGAGTTATATTTTTCAATGAATGTGTC
AATTTCCTCTGAATATATATGTATTGGCATAAAATTGTTACAATATCTTTGTATTTCTTTTCAATATAAG
TAGGATCTATAGCAATGTCTATGTATCTGTTATGATTATTTGCATTTTATCTTTATTTTATTGTATTTTT
TTTTGAGATGGAGTCTCACTCTGTCACCCAGGCTGGAGTGGCTGGAGTACAGTGGCACGATCTTGGCTCA
CTGCAATCTCTGCCTCCCAGGTTCAAGAGATTCTCCTGCCTCAGCCTCCCAGGTAGCTGGGATTACAGAT
GCCCACCACCATTCCTGGCTAATTTTTTTAATTTTTTGTGGAGACGAGGTTTCGCCATGCTGGCTAGGCT
GGTCTCAAACCCCTGACCTGAGGTGATCTACCTGCCTCAGCCTCCCAAAGTGCTGGGATTACAGGCATGA
GCCACTGTGCTTGGCCCATCTCTTTTTTTCTTAATCAATCTTGCAAAGGATTTGTCCATTTTATTAAATT
TTTCCAAAAACTTCTTTTTGGTGTTTACTAACCTCTATGATTACATTTATTTATATCTGTTCCTGTCTTT
ATCATTTCTTGTATCGATCTTCTGTGGGTTTAATTGGCCATTCTTTTTCTAGCTTCTTTTAATTGACATT
TAGAGAACTGATTTTCTTCCTTCCTTCTTTGCTAATATAATCATTTAAAGTTATAATTTTTATTTAAGAA
CTATTTCAGCTGCATACCACAGGTTTGGATATGTATCTTTTCCCACCAATTCAAAATAGGTTTTTCATAT
CCAATTCAAAATATTTTATAATTTTCATTGTGACTTCTTCGACTCATGGTTTATTTATAAGTGTGTGGTT
TAATTCCTAAATATTTGAGTATTTTAAAAAGCTATACTCCTGTAAGTGATTTATAGTTTAATTCCACTGT
ATCCAGAAATATACTTTTATATGAGTTCAATCTTTTGAAATTTGTTAAGATTTTATTTTATGATCCAGTT
TATGATTTAGAATGGTAAATGTATTATGTGCACTAGAAATAATGTGGTTTTTACAGTTGATTAGTGTATC
ATTAGGTCAGGATATGAATCTTCTTTGTATTTTCTGTATTCTCACTAATATTTTGCCCTTTTAGAAGGAA
ATAACAAAGATCAGAATAGAAATAAATGACATAGAGAGTAGAAAAGCAAATAGATCAATGAAACTCAAAA
GTTAATTTTTGTGAAATGATACAATTTCCAAACTTTAGCTAAAATAACTAAGAAAAATACAGAGAAGACT
CAAAATCAGAAATGAGAGGAGACACGACAACTGATAACATACAAGTACAAAGGATCATAAGAGCTACATT
CCTTTGTGTTACATTGTTTTCTCTTTACATTCTGATTGTCTCTCGCTTGCTCGCTGTCTTCTTTTAAAGT
TTTGTTTCAGAAGAACTAAAAAAATCTAGGTTTAAAATGTACAGATATCCTTTGTAACTTAGCATTAGGC
AAAATTGTCTTAGAGAACTCAATTACTTTTCAAGTAAAAAATGATAAATTGAATTTCATCAAAATTAAAG
ATTAACTCAGTTTGTTAAACATTAGGAAAACAGGCAAACCATGACTAGGAGAAAAATATTTGCAAATATT
GGCCTGCATCTGTCAAAGGAT (SEQ ID NO: 1)
MBL Polypeptide
Primary amino-acidic sequence of MBL. Underlined the leader sequence.
MSLFPSLPLLLLSMVAASYSETVTCEDAQKTCPAVIACSSPGINGFPGKD
GRDGTKGEKG EPGQGLRGLQGPPGKLGPPGNPGPSGSPGPKGQKGDPGKS
PDGDSSLAASERKALQTEMARIKKWLTFSLGKQVGNKFFLINGEIMTFEK
VKALCVKFQASVATPRNAAENGAIQNLIKEEAFLGITDEKTEGQFVDLTG
NRLTYTNWNEGEPNNAGSDEDCVLLLKNGQWNDVPCSTSHLAVCEFPI [SEQ ID N. 2]
MBL haplotype
ATCCCCGCATTGA [SEQ ID N. 3]
MBL haplotype:
AGATCCCCGCGCGTGCAACGGCTGCGGA [SEQ ID N. 4],

### EXAMPLES

### Example 1

### Materials and Methods

### Recombinant Proteins and Antibodies

Recombinant SARS-COV-2 proteins used in this study are listed in the following Table 10.

TABLE 10
List of recombinant SARS-CoV-2 proteins used in this study.

| Proteins | Host | Cat | Company |
|---|---|---|---|
| SARS-CoV-2 S1 protein, | HEK293 | S1N-C52H4 | ACROBiosystems |
| His Tag | | | |
| SARS-CoV-2 S2 protein, | HEK293 | S2N-C52H5 | ACROBiosystems |
| His Tag | | | |
| SARS-CoV-2 S protein, | HEK293 | SPN-C52H8 | ACROBiosystems |
| His Tag, active trimer | | | |
| SARS-CoV-2 | HEK293 | NUN-C5227 | ACROBiosystems |
| Nucleocapsid | | | |
| protein, His Tag | | | |
| SARS-CoV-2 Envelope | E. coli | ENN-C5128 | ACROBiosystems |
| protein. GST, | | | |
| His Tag | | | |
| Biotinylated SARS-CoV-2 | HEK293 | SPN-C82E3 | ACROBiosystems |
| S protein, His Tag, | | | |
| active trimer | | | |
| SARS-CoV-2 (2019-nCoV) | HEK293 | 40592-V08H | Sino Biological |
| Spike RBD, His Tag | | | |
| SARS-CoV-2 (2019-nCoV) | Insect | 40589- | Sino Biological |
| Spike S1 + S2 ECD, | cells | V08B1 | |
| His Tag | | | |

Recombinant His-Tag SARS-COV-2 proteins from HEK293 cells were purchased from ACROBiosystems. Recombinant His-Tag SARS-COV-2 RBD and S1+S2 Ectodomain (ECD, expressed in insect cells) were from SinoBiological. Recombinant hPTX3 from CHO cells was produced in house, as previously described 51. Recombinant human MBL, Ficolin-1, Ficolin-2 and Ficolin-3 were from Biotechne. Purified human C1q was from Complement Technology, purified C-reactive protein (CRP) was from Millipore and purified Serum Amyloid Protein (SAP) was purchased from Abcam. Rabbit anti-PTX3 antibody was produced in house⁵¹, rabbit anti-MBL Ab was purchased from Abcam. Anti-C1q polyclonal antibody was purchased from Dako. Anti-CRP and anti-SAP antibodies were from Merck.

### Binding of Humoral Pattern Recognition Molecules to SARS-COV-2 Proteins

Recombinant His-Tag SARS-COV-2 proteins were immobilized at different concentrations (ranging from 6.25 to 50 pmol/mL) on 96-well Nickel coated plates (Thermo Fisher Scientific, USA) for 1 hour at room temperature. Plates were then blocked for 2 hours at 37° C. with 200 μL of 2% BSA diluted in 10 mM Tris-HCl buffer, pH 7.5, containing 150 mM NaCl, 2 mM CaCl₂ and 0.1% Tween-20 (TBST-Ca²⁺). Following blocking, plates were washed three times with TBST-Ca²⁺ and incubated 1 hour at 37° C. with 100 μL PTX3 (4 μg/mL in TBST-Ca²⁺), MBL (2 μg/mL in TBST-Ca²⁺), C1q (4 μg/mL in TBST-Ca2), CRP (3 μg/mL in TBST-Ca2) and SAP (4 μg/mL in TBST-Ca²⁺). After washes, plates were incubated 1 hour at 37° C. with specific primary antibodies, followed by the corresponding HRP-conjugated secondary antibodies. Both primary and secondary antibodies were diluted in TBST-Ca2 buffer. After development with the chromogenic substrate 3,3′,5,5′-tetramethylbenzidine (TMB, Thermo Fisher Scientific, USA), binding was detected by absorbance reading at 450 nm on a Spectrostar Nano Microplate Reader (BMG Labtech, Germany). Values from blank wells were subtracted from those recorded at sample wells.

In another set of experiments, 100 μL of 2 μg/mL rhMBL, Ficolin-1, Ficolin-2, and Ficolin-3 in PBS were immobilized on 96-well Nunc Maxisorp Immunoplates (Costar, USA) overnight at 4° C. Plates were blocked with 200 μL of 2% BSA-TBST-Ca²⁺ for 2 hours at 37° C. After washes, 100 μL of biotinylated SARS-COV-2 S protein was added at different concentrations to the plates for 1 hour at 37° C. Following washes, HRP-conjugated streptavidin (1:10000, Biospa) was incubated for 1 hour at 37° C. Specific binding was detected by TMB development, as described above.

For competition-based experiments, biotinylated SARS-COV-2 S protein was captured on 96-wells Neutravidin coated plates for 1 hour at 37° C. Plates were then incubated for 1 hour at 37° C. with 100 μL rhMBL (0.625 ng/mL) alone or in the presence of various concentrations of D-Mannose or N-Acetyl-glucosamine (Sigma Aldrich). Bound MBL was detected by incubation with rabbit anti-MBL antibody, followed by HRP-conjugated secondary antibody and TMB development as described above.

### Cell Lines

The Vero cell line was obtained from the Istituto Zooprofilattico of Brescia, Italy, and was maintained in Eagle's minimum essential medium (EMEM; Lonza) supplemented with 10% fetal bovine serum (FBS; Euroclone) and penicillin-streptomycin (complete medium).

The human lung epithelial Calu3 cell line was obtained from NovusPharma. Cells were grown in EMEM supplemented with 20% FBS and penicillin-streptomycin (complete medium).

### SARS-COV-2 Viral Isolate

The SARS-COV-2 isolate (GISAID accession ID: EPI_ISL_413489) was obtained from the nasopharyngeal swab of a mildly symptomatic patient by inoculation of Vero cells as described in^{64,65} (informed consent of the patient was obtained). A secondary viral stock was generated by infection of adherent Vero cells seeded in a 25 cm² tissue culture flask with 0.5 ml of the primary isolate diluted in 5 ml of complete medium. Three days after infection, the supernatant was harvested and, after centrifugation, passed through a 0.45μ filter. Aliquots of the secondary SARS-COV-2 isolate were maintained at −80 ° C. A plaque-forming assay was performed to determine viral titers.

### Infections

Calu3 cells were seeded in 48-well plates (Corning) at the concentration of 5×10⁴ cell/well in complete medium 24 h prior to infection. Ten-fold serial dilutions of MBL (from 0.01 to 10 μg/ml) were incubated for 1 h with aliquots of SARS-COV-2 containing supernatant to obtain a multiplicity of infection (MOI) of either 0.1 or 1 before incubation with Calu3 cells (Virus+MBL). After 48 and 72 h post-infection, cell culture supernatants were collected and stored at −80 ° C. until determination of the viral titers by a plaque-forming assay in Vero cells. Virus incubation with MBL was also combined with incubation of target cells. Briefly, virus incubation with MBL was performed as described above whereas Calu3 cells were incubated with 10-fold serial dilutions of MBL (from 0.01 to 10 μg/ml). After 1 h, virus suspensions incubated with serial dilutions of MBL were added to MBL-treated cells (Virus+Cells+MBL). After 48 and 72 h post-infection, cell culture supernatants were collected and stored at −80 ° C. until determination of the viral titers by a plaque-forming assay in Vero cells.

### Plaque-Forming Assay

In order to measure the virus titer of the viral stocks, a plaque-forming assay was optimized in Vero cells. Briefly, confluent Vero cells (1.5×106 cell/well) seeded in 6-well plates (Corning) were incubated in duplicate with 1 ml of EMEM supplemented with 1% FBS containing 10-fold serial dilutions of SARS-COV-2 stock. After 1 h of incubation, the viral inoculum was removed and methylcellulose (Sigma; 1 ml in EMEM supplemented with 5% FBS) was overlaid on each well. After 4 days of incubation, the cells were stained with 1% crystal violet (Sigma) in 70% methanol. The plaques were counted after examination with a stereoscopic microscope (SMZ-1500; Nikon Instruments) and the virus titer was calculated in terms of plaque forming units (PFU)/ml.

In order to determine the viral titers of the supernatant collected from Calu3 cells at 48 and 72 h post-infection, confluent Vero cells (2.5×105 cell/well) were seeded in 24-well plates (Corning) 24 h prior to infection. Then, cells were incubated with 300 μl of EMEM supplemented with 1% FBS containing serially diluted (1:10) virus-containing supernatants. The plaque-forming assay was performed as described above.

### Statistical Analysis

Prism GraphPad software v. 8.0 (www.graphpad.com) was used for the statistical analyses. Comparison among groups were performed using the two-way analysis of variance (ANOVA) and the Bonferroni's correction.

### RESULTS

### Mannose Binding Lectin (MBL) Interacts With SARS-COV-2 Viral Proteins

Inventors investigated the binding of humoral innate immunity molecules (PTX3, CRP, SAP, C1q and MBL) to SARS-COV-2 proteins by solid phase binding assay.

They first analysed pentraxins, and as shown in FIG. 1, and did not observe any specific binding of CRP and SAP to the viral proteins tested (SARS-COV-2 S1 protein, SARS-COV-2 S2 protein, SARS-COV-2 S protein active trimer, SARS-COV-2 Nucleocapsid protein, SARS-COV-2 Envelope protein) (FIG. 1A, 1B). In contrast, PTX3 bound specifically and in a dose-response manner to Nucleocapsid protein, one of the most abundant proteins of SARS-COV-2¹⁷ (FIG. 1C).

Inventors next investigated the interaction between the lectins C1q or MBL and the viral proteins. As shown in FIG. 2A, C1q did not interact with any protein tested. In contrast, human MBL bound to SARS-COV-2 Spike protein (active trimer), but not to single SARS-COV-2 Spike subunits S1 (containing the RBD domain) and S2. These results indicate that a native-close structure of the SARS-COV-2 Spike protein is indispensable for MBL recognition. Since MBL shares with ficolins a conserved domain involved in the interaction with glycosidic moieties of microbial structures, the Carbohydrate Recognition Domain (CRD), inventors also investigated the interaction between Ficolin-1, -2 or -3 and SARS-COV-2 Spike protein. As shown in FIG. 2C, in contrast with MBL, ficolins did not interact with SARS-COV-2 S protein.

The SARS-COV-2 Spike protein plays the most important roles in viral attachment, fusion and entry, and serves as a target for development of antibodies, entry inhibitors and vaccines. The receptor-binding domain (RBD) in SARS-COV-1 and SARS-COV-2 Spike protein is the domain responsible of the interaction with human and bat Angiotensin-Converting Enzyme 2 (ACE2), which are transmembrane proteins acting as SARS-COV-2 receptors, with the cooperation of other host factors such as the TMPRSS2 protease^{75,76}. To dissect the interaction of MBL with SARS-COV-2 Spike protein, inventors compared the interaction with the S active trimer, the RBD, and the S1+S2 extracellular (ECD) domain (ectodomain). As shown in FIG. 2D, MBL did not bind to the RBD, confirming the results obtained using the Subunit S1, and bound to the S1+S2 ECD domain, although with lower affinity in comparison with the trimeric protein. These data indicate that different sites in the SARS-COV-2 Spike trimeric protein are necessary for the interaction with MBL and that the RBD is not involved in the interaction.

The SARS-COV-2 Spike protein is highly glycosylated, as recently described¹⁸. Inventors evaluated if MBL interacts with the viral protein through its CRD. First, they analyzed the binding of MBL to the spike proteins in the presence or absence of Ca²⁺. To this aim, MBL-coated plates were incubated with different concentrations of biotinylated SARS-COV-2 Spike protein diluted in TBS with or without Calcium ions. The binding was detected by addition of HRP-conjugated streptavidin and developed as described above. FIG. 3A shows that removal of Ca²⁺ completely prevented the interaction of MBL with SARS-COV-2 Spike protein.

Next, they set up a solution-based competition assay to further investigate the role of the MBL CRD in the recognition of the spike protein. MBL, alone or in presence of two well-known ligands of the lectin, D-Mannose and N-Acetyl-Glucosamine, was incubated over biotinylated SARS-COV-2 Spike proteins captured on a neutravidin-coated plate. Bound MBL was detected with an anti-MBL antibody. Data depicted in FIG. 3B show that both the monosaccharides inhibited MBL binding to the spike proteins, thus confirming the Ca^{2 +}-dependent interaction between the MBL lectin domain and the glycosidic sites exposed by the Spike protein.

### MBL prevents the Viral Replication and Cytopathic Effects in Epithelial Cells

In order to evaluate whether MBL affected virus replication, inventors optimized an assay that allowed the evaluation of the whole virus life cycle.

SARS-COV-2 (MOI=0.1 and 1) was preincubated in complete medium containing different concentrations of MBL (0.01-10 μg/mL) before incubation with Calu3 cells. After 48 and 72 h, the infectivity of SARS-COV-2 present in cell culture supernatants was determined by a plaque-forming assay in monkey-derived Vero cells. Vero cells are a handy cell line used worldwide as it is devoid of the interferon (IFN) response 20 and, for this reason, highly supportive of virus replication.

As shown in FIGS. 4 and 5, MBL showed a concentration-dependent inhibition of SARS-COV-2 infection of Calu3 cells at MOI 0.1 and 1, that was statistically significant at 72 h after infection. When both virus and cells were pre-incubated with the same concentrations of MBL (0.01-10 μg/mL), the antiviral activity increased significantly from 0.1 μg/ml to the top concentration of 10 μg/ml, 72 h post-infection.

### Examples 2-8

### Material and Methods

### Patient Cohorts and Ethical Approvals

Approvals were obtained from the relevant ethics committees (Humanitas Clinical and Research Center, reference number, 316/20; the University of Milano-Bicocca School of Medicine, San Gerardo Hospital, reference number, 84/2020). The requirement for informed consent was waived.

For genetic association analyses, we investigated 2,000 individuals. These included: i) 332 patients with severe COVID-19, defined as hospitalization with respiratory failure and a confirmed SARS-COV-2 viral RNA PCR test from nasopharyngeal swabs. Patients were recruited from intensive care units and general wards at two hospitals in the Milan area [Humanitas Clinical and Research Center, IRCCS, Rozzano, Italy (140 patients); San Gerardo Hospital, Monza, Italy (192 patients)]; ii) 1,668 controls from Italian population with unknown COVID-19 status.

MBL plasma concentrations were analyzed in a cohort of 40 patients including all males and non-pregnant females, 18 years of age or older, admitted to Humanitas Clinical and Research Center (Rozzano, Milan, Italy) between March and April, 2020 with a laboratory-confirmed diagnosis of COVID-19.

### Recombinant Proteins and Antibodies

Recombinant SARS-COV-2 proteins used are listed in Table 9. Recombinant human PTX3 and its domains were produced in-house, as described⁵¹. Recombinant human SP-A was from Origene. Recombinant human MBL, Collectin-12, Ficolin-1, Ficolin-2, Ficolin-3 were from Biotechne. Other recombinant preparations of Ficolin-2 were from Abnova, Origene, and SinoBiological. SP-D was from Biotechne and SinoBiological. Recombinant human Collectin-10 (CL-L1) and Collectin-11 (CL-K1) were from Abnova. Recombinant human CL-K1 or CL-L1/CL-K1 heterocomplexes were also expressed and purified as described39. Purified human C1q was from Complement Technology, purified CRP was from Millipore and purified SAP from Abcam. Rabbit anti-PTX3 antibody (1:5000) was produced in-house⁵¹, rabbit anti-MBL Ab (1:5000) was from Abcam. Anti-C1q polyclonal antibody (1:5000) was from Dako. Anti-CRP (1:5000) and anti-SAP (1:5000) antibodies were from Merck. Mouse monoclonal IgG anti-human CL-K1 (clone Hyb-15, (1:2000) and mouse monoclonal IgG anti-human Ficolin-2 (clone FCN219) were produced in-house^{39,52}. The following secondary antibodies were used: HRP-linked donkey anti-rabbit IgG (GE Healthcare, 1:5000); HRP-linked sheep anti-mouse IgG (GE Heathcare, 1:5000).

### Binding of Humoral PRM to SARS-COV-2 Proteins

Recombinant His-Tag SARS-COV-2 proteins were immobilized (concentrations ranging from 6.25 to 50 pmol/mL) on 96-well Nickel coated plates (Thermo Fisher Scientific) for 1 h at 20° C. Plates were then blocked for 2 h at 37° C. with 200 μL of 2% BSA in 10 mM Tris-HCl buffer, pH 7.5, containing 150 mM NaCl, 2 mM CaCl₂ and 0.1% Tween-20 (TBST-Ca²⁺). Then, plates were washed three times with TBST-Ca²⁺ and incubated for 1 h at 37° C. with 100 μL PTX3 (4 μg/mL-12 nM in TBST-Ca²⁺), MBL (1-2 μg/mL-3.4-6.7 nM in TBST-Ca²⁺), C1q (4 μg/mL-10 nM in TBST-Ca²⁺), CRP (3 μg/mL-25 nM in TBST-Ca²⁺) and SAP (4 g/mL-32 nM in TBST-Ca²⁺), Ficolin-2 (1 μg/mL-2.5 nM in TBST-²⁺), CL-K1 (1 μg/mL-6.7 nM in TBST-Ca²⁺). After washes, plates were incubated for 1 h at 37° C. with specific primary antibodies, followed by HRP-conjugated secondary antibodies diluted in TBST-Ca²⁺ buffer. After development with the chromogenic substrate 3,3′,5,5′-tetramethylbenzidine (TMB, Thermo Fisher Scientific), binding was detected by absorbance reading at 450 nm on a Spectrostar Nano Microplate Reader (BMG Labtech). Values from blank wells were subtracted from those recorded at sample wells.

For binding experiments of SARS-COV-2 S protein to CL-L1/CL-K1 heterocomplexes, S protein was immobilized on a 96-well Nunc Maxisorp plate. MBL (1μg/mL-3.4 nM) or CL-L1/CL-K1 heterocomplexes (1μg/mL-3.4 nM) in TBST-Ca²⁺ were then incubated for 1 h at 37° C., followed by specific primary antibodies³⁹, HRP-conjugated secondary antibodies and TMB development.

In other experiments, 100 μL of 2 μg/mL rhMBL (6.7 nM), CLP-1 (6.7 nM), Ficolin-1 (5 nM), Ficolin-2 (5 nM), and Ficolin-3 (3 nM), SP-A (3 nM) or SP-D (3.4 nM) in PBS were immobilized on 96-well Nunc Maxisorp Immunoplates (Costar, USA) overnight at 4° C. Plates were blocked with 200 μL of 2% BSA-TBST-Ca²⁺ for 2 h at 37° C. Biotinylated SARS-COV-2 S protein was added for 1 h at 37° C., followed by HRP-conjugated streptavidin (1:10000, Biospa) for 1 h at 37° C. and TMB development.

For competition-based experiments, biotinylated SARS-COV-2 S protein was captured on 96-wells Neutravidin coated plates for 1 h at 37° C. Plates were incubated for 1 h at 37° C. with 100 μL rhMBL (0.25 μg/mL-0.83 nM) alone or in the presence of D-mannose or N-acetyl-glucosamine, or D-glucose (Sigma Aldrich). Bound MBL was detected by incubation with rabbit anti-MBL antibody, followed by HRP-conjugated secondary antibody and TMB development.

For PTX3/SARS-COV-2 Nucleocapsid interaction studies, PTX3 and its recombinant domains were immobilized on a 96-well Nunc Maxisorp plate. Then, biotinylated SARS-COV-2 Nucleocapsid protein was added for 1 h at 37° C., followed by HRP-conjugated streptavidin.

### Surface Plasmon Resonance (SPR) Studies

SPR analyses were carried out at 25 ° C.on a Biacore 8K instrument (GE Healthcare). MBL was immobilized on the surface of a CM5 sensor chip through standard amine coupling. Briefly, after activation of the surface with a mixture of 1-Ethyl-3-(3-dimethylaminopropyl) carbodiimide hydrochloride and N-Hydroxysuccinimide, MBL was diluted at 50 nM in 10 mM sodium acetate buffer, pH 4.5 and injected over the surface (flow rate 10 μl/min). Free activated sites were blocked by flowing 1 M Ethanolamine, pH 8.5. Final MBL immobilization levels were around 4500 Resonance Units (RU, with 1 RU=1 pg/mm²). A second surface was prepared without any ligand, and used as reference. Recombinant RBD and trimeric Spike were produced in Expi293 cells and purified as reported⁵³. Increasing concentration of SARS-COV-2 RBD or Spike protein (2.5, 7.4, 22, 67, 200 and 600 nM) were injected using a single-cycle kinetics setting (flow rate 30 μl/min); dissociation was followed for 10 minutes. The running buffer was 10 mM Tris-buffered saline, pH 7.4, containing 150 mM NaCl, 2 mM CaCl₂ and 0.005% Tween-20. The interaction was also analyzed using the running buffer without CaCl₂. Analyte responses were corrected for unspecific binding and buffer responses through the use of reference channels. Binding kinetics were determined by fitting of the experimental curves with the Langmuir 1:1 model according to standard procedures; data analyses were performed with Biacore™ Insight Evaluation Software v2.0.15.12933. In the presence of CaCl₂, trimeric Spike bound to MBL with Kₐ(1/Ms)=2.1e⁺⁴, Kₐ(1/s)=7.3e⁻4 and K_{D}=34 nM.

### Computational Modeling of the MBL SARS-COV-2 Spike Interaction

The model of the MBL trimer (UniProt⁵⁴ P11226) was created starting from the crystal structure of human mannose binding protein⁵⁵ (PDB code 1HUP). The N-terminus of MBL was modeled as collagen, based on the template crystal structure of collagen triple helix model⁵⁶ (PDB code 1K6F). The binding site of mannose molecules was determined aligning the MBL structure to the crystal structure of rat mannose protein A⁵⁷ (PDB code 1KX1). Reference distances (˜40 Å) between mannose molecules were computed in PYMOL.

Putative binding sites of MBL were determined identifying all triplets of N-glycosylation sites at a distance between 35 Å and 50 Å in the closed state SARS-COV-2 Spike protein⁵⁸. Distances were computed using the program ALMOST⁵⁹.

### Pseudotyped Virus Production

Human 293T cells were transfected with a lentiviral vector expressing the Green Fluorescent Protein (GFP) under the control of a human Phosphoglycerate Kinase promoter (PGK)⁶⁰ and a pCMV expressing vector containing the SARS-COV-2 Spike sequence (accession number MN908947) that was codon-optimized for human expression and contained a deletion at the 3′ end aimed at deleting 19 amino acid residues at the C-terminus. An HIV gag-pol packaging construct and a rev-encoding plasmid were co-transfected by calcium phosphate for the production of infectious viral particles. 16 h after transfection, the medium was replaced and 30 h later, supernatant was collected, filtered through 0.22 μm pore nitrocellulose filter and viral particles were pelleted by ultracentrifugation. As control, lentivirus particles were pseudotyped with the VSV-g glycoprotein that allows a high efficiency infection independently of binding to ACE2.

### Pseudotyped Lentivirus Binding Assay

96-well Nunc Maxisorp Immunoplates (Costar) were coated with 100 μL of rhMBL (3 and 1 μg/mL-10 and 3.4 nM in PBS). After overnight incubation, plates were blocked with 2% BSA in TBST-Ca²⁺ for 1 h at 37° C., washed and incubated for 1 h with 100 μL of SARS-COV-2 Spike protein-pseudotyped lentivirus or VSV-pseudotyped lentivirus (from 0.1 to 1 μg/mL in TBST-Ca²⁺). After washing, bound pseudotyped virus particles were lysed with 0.5% Triton X-100 and HIV p24 core protein was detected by ELISA (Perkin Elmer).

### Complement Deposition Assay

100 μL of SARS-COV-2 Spike protein (either active trimer or non-covalent trimer, 1 μg/mL in PBS) were captured on 96 well plates overnight at 4° C. After washing, wells were incubated for 1 h at 37° C. with either 10% normal human serum (NHS, ComplemenTech Inc, USA), 10% C1q-depleted serum (C1qDHS), 10% C4-depleted serum (C4DHS) reconstituted or not with 25 μg/mL purified C4 (Calbiochem). 10% heat-inactivated human serum (30′ at 56° C. , HI-NHS) and 10% C3-depleted serum (C3DHS) were used as negative control. Sera were diluted in 10 mM Tris-buffered saline containing 0.5 mM MgCl₂, 2 mM CaCl₂ and 0.05% Tween-20, also used as washing buffer. For MBL immunodepletion, 10% NHS was incubated overnight with 0.6 μg/mL rabbit anti-MBL antibody. Bound MBL-antibody complexes were separated by Dynabeads Protein G (Thermo Fisher Scientific), and the supernatant (termed MBL-ID) was used in the assay (final concentration, 10%). C5b-9 deposition was assayed by incubation for 1 h at 37° C. with rabbit anti-sC5b-9 antibody (ComplemenTech Inc.) diluted 1:2000 in washing buffer⁶¹, followed by specific HRP-conjugated secondary antibody and TMB development.

### Cell Lines

Vero and Vero E6 cell lines were obtained from the Istituto Zooprofilattico of Brescia, Italy, and ATCC, respectively, and maintained in Eagle's minimum essential medium (EMEM; Lonza) with 10% fetal bovine serum (FBS; Euroclone) and penicillin-streptomycin (complete medium).

Human embryonic kidney 293T cells containing the mutant gene of SV40 Large T Antigen (ATCC code CRL-3216), were cultured as described⁶².

The human lung epithelial Calu-3 cell line was obtained from NovusPharma and grown in EMEM supplemented with 20% FBS and penicillin-streptomycin (complete medium).

### Human Bronchial Epithelial Cells (HBEC)

Isolation, culture, and differentiation of primary human bronchial epithelial cells (HBECs) were performed as reported⁶³. In brief, cells were obtained from mainstem human bronchi, derived from individuals undergoing lung transplant from three donors (BE37, BE63 and BE177). Epithelial cells were detached by overnight treatment of bronchi with protease XIV and then were cultured in a serum-free medium (LHC9 mixed with RPMI 1640, 1:1) containing supplements⁶³. The collection of bronchial epithelial cells was approved by the Ethics Committee of the Istituto Giannina Gaslini following Italian Ministry of Health guidelines (registration number: ANTECER, 042-09/07/2018). Patients provided informed consent to the study.

To obtain differentiated epithelia, cells were seeded at high density (5×10⁵ cell/snapwell) on 12-mm diameter porous membranes (Snapwell inserts, Corning, code 3801). After 24 hours, the serum-free medium was removed from both sides and, on the basolateral side only, replaced with Pneumacult ALI medium (StemCell Technologies) and differentiation of cells (for 3 weeks) was performed in air-liquid interface (ALI) condition.

### Entry Assay With SARS-COV-2 Spike-Pseudotyped Lentivirus Particles

293T cells were engineered to overexpress the SARS-COV-2 entry receptor by transduction of a lentiviral vector expressing ACE2 (provided by M. Pizzato, University of Trento). The entry assay was optimized in 96-well plate by seeding 5×10⁴ ACE2 overexpressing 293T cells/well. 24 h later, cells and SARS-COV-2 Spike-pseudotyped lentivirus stock (1:500) were incubated with serial dilutions of soluble PRM for 30 min. The SARS-COV-2 Spike-pseudotyped was added to the cells and 48 h later, cells were detached with accutase, fixed and analyzed for GFP expression by cytofluorimetry.

### SARS-COV-2 Viral Isolates

Viral isolation from clinical samples and use for research purposes was approved by San Raffaele Hospital IRB within the COVID-19 Biobanking project “COVID-Biob” (34/int/2020 19 March 2020. ClinicalTrials.gov Identifier: NCT04318366). Each patient provided informed consent.

SARS-COV-2 isolates were obtained from nasopharyngeal swabs: 1) B. 1 lineage with the Spike D614G mutation (GISAID accession ID: EPI_ISL_413489) from a mildly symptomatic patient by inoculation of Vero E6 cells^{64,65}; 2) South African B.1.351 (β) lineage (GISAID accession ID: EPI_ISL_1599180) from an Italian 80-year-old male patient; 3) B.1.1.7 (α) lineage (GISAID accession ID: EPI_ISL_1924880) from an Italian 58-year-old female patient; 4) P.1 (γ) lineage (GISAID accession ID: EPI_ISL_1925323) from an Italian 43-year-old female patient; 5) B.1.617.2 (δ) lineage (GISAID accession ID: EPI_ISL_4198505) from an Italian 50-year-old male patient. Secondary viral stocks were generated by infection of Vero E6 cells, maintained at −80 ° C. and titered by a plaque-forming assay.

### Infections

Calu-3 cells were seeded in 48-well plates (Corning) at the concentration of 5×10⁴ cell/well in complete medium 24 h prior to infection. Ten-fold serial dilutions of MBL (from 0.01 to 10 μg/ml-0.034-34 nM) were incubated for 1 h with aliquots of SARS-COV-2 containing supernatant to obtain a multiplicity of infection (MOI) of either 0.1 or 1 before incubation with Calu-3 cells (Virus+MBL). Virus incubation with MBL was also combined with incubation of target cells. Briefly, both virus and Calu-3 cells were incubated with 10-fold serial dilutions of MBL (from 0.01 to 10 μg/ml-0.034-34 nM). After 1 h, virus suspensions incubated with serial dilutions of MBL were added to MBL-treated cells (Virus+Cells+MBL). In both cases, after 48 and 72 h PI, cell culture supernatants were collected and stored at −80 ° C.until determination of the viral titers.

48 h before infection, the apical surface of HBEC was washed with 500 μl of HBSS for 1.5 h at 37° C., and the cultures were moved into fresh ALI medium. Immediately before infection, apical surfaces were washed twice to remove accumulated mucus with 500 μl of HBSS for 30 min at 37° C. PTX3 or MBL were added to the apical surface for 1 h prior to the addition of 100 ul of viral inoculum at a MOI of 1. HBEC were incubated for 2 h at 37° C. Viral inoculum was then removed and the apical surface of the cultures was washed three times with 500 μl of PBS. Cultures were incubated at 37° C. for 72 h PI. Infectious virus produced by the HBEC was collected by washing the apical surface of the culture with 100 μl of PBS every 24 h up to 72 h PI. Apical washes were stored at −80° C. until analysis and titered by plaque assay. At 72 h PI, cells were fixed in 4% paraformaldehyde for immunofluorescence analysis.

All infection experiments were performed in a BSL-3 laboratory (Laboratory of Medical Microbiology and Virology, Vita-Salute San Raffaele University).

Measurements were taken from distinct samples.

### Plaque-Forming Assay

The viral stock titer was measured by a plaque-forming assay in Vero cells. Briefly, confluent Vero cells (1.5×10⁶ cell/well) seeded in 6-well plates (Corning) were incubated in duplicate with 1 ml of EMEM with 1% FBS containing 10-fold serial dilutions of SARS-COV-2 stock. After 1 h the viral inoculum was removed and methylcellulose (Sigma; 1 ml in EMEM with 5% FBS) was overlaid on each well. After 4 days, cells were stained with 1% crystal violet (Sigma) in 70% methanol. Plaques were counted with a stereoscopic microscope (SMZ-1500; Nikon Instruments) and the virus titer was calculated as plaque forming units (PFU)/ml.

To determine the viral titers of the supernatant collected from Calu-3 and HBEC cells, confluent Vero cells (2.5×10⁵ cell/well) were seeded in 24-well plates (Corning) 24 h prior to infection. Then, cells were incubated with 300 μl of EMEM with 1% FBS containing serially diluted (1:10) virus-containing supernatants. The plaque-forming assay was performed as described above.

### Chemokine Quantification

Half of the ALI medium (1 ml) was collected from each well of the lower chamber every 24 h PI and replaced with fresh ALI medium. The harvested medium was stored at −80 ° C.until analysis. Prior to chemokine quantification, 250 μl of medium was treated with 27 μl of Triton X-100 and heated for 30 min at 56 ° C.to inactivate SARS-COV-2 infectivity.

Chemokines (IL-8 and CXCL5) were quantified by ELISA (Quantikine ELISA kits, code DY208, DY254, R&D Systems).

### Confocal and STED Super-Resolution Microscopy

After 4% PFA fixation, HBEC cultures were incubated for 1 h with PBS and 0.1% Triton X-100 (Sigma-Aldrich), 5% normal donkey serum (Sigma-Aldrich), 2% BSA, 0.05% Tween (blocking buffer). Cells were then incubated for 2 h in blocking buffer with the following primary antibodies: mouse anti-cytokeratin 14 (Krt14) (#LL002; 1 μg/ml; cat. N° 33-168, ProSci-Incorporated); rabbit polyclonal anti-Spike protein (944-1218aa) (2 μg/ml; cat. N° 28867-1-AP, Proteintech®); rat anti-MBL (#8G6; 1μg/ml; cat. N° HM1035, Hycult®Biotech) and rat anti-MBL (#14D12; 1 μg/ml; cat. N° HM1038, Hycult® Biotech). After washing with PBS and 0.05% Tween, cells were incubated for 1 h with the following species-specific cross-adsorbed secondary antibodies form Invitrogen-ThermoFisher Scientific: donkey anti-rabbit IgG Alexa Fluor® 488 (1 μg/ml; cat. N° A-21206); donkey anti-rat IgG Alexa Fluor® 594 (1 μg/ml; cat. N° A-21209); donkey anti-mouse IgG Alexa Fluor® 647 (1 μg/ml; cat. N° A-31571). 4′,6-diamidino-2-phenylindole (DAPI) (Invitrogen) was used for nucleus staining. Cells were mounted with Mowiol® (Sigma-Aldrich) and analyzed with a Leica SP8 STED3× confocal microscope system equipped with a Leica HC PLAPO CS2 63×/1.40 oil immersion lens. Confocal images (1.024×1.024 pixels) were acquired in XYZ and tiling modality (0.25 μm slice thickness) and at 1 Airy Unit (AU) of lateral resolution (pinhole aperture of 95.5 μm) at a frequency of 600 Hz in bidirectional mode. Alexa Fluor 488® was excited with a 488 nm argon laser and emission collected from 505 to 550 nm. Alexa Fluor 594® was excited with a 594/604nm-tuned white light laser and emission collected from 580 to 620nm. Alexa Fluor 5647® was excited with a 640/648nm-tuned white light laser and emission collected from 670 to 750nm. Frame sequential acquisition was applied to avoid fluorescence overlap. A gating between 0.4 and 7 ns was applied to avoid collection of reflection and autofluorescence. 3D STED analysis was performed using the same acquisition set-up. A 660 nm CW-depletion laser (30% of power) was used for excitations of Alexa Fluor 488® (Spike signal) and Alexa Fluor 594® (MBL signal). STED images were acquired with a Leica HC PL APO 100×/1.40 oil STED White objective at 572.3 milli absorption unit (mAU). CW-STED and gated CW-STED were applied to Alexa Fluor 488® and Alexa Fluor 594®, respectively. Confocal images were processed, 3D rendered and analyzed as colocalization rate between Spike and MBL with Leica Application Suite X software (LASX; version 3.5.5.19976) and presented as medium intensity projection (MIP). STED images were de-convolved with Huygens Professional software (Scientific Volume Imaging B. V.; version 19.10) and presented as MIP.

### Genetic Analysis and Imputation

Details on DNA extraction, array genotyping and quality checks are reported elsewhere12.50. Genetic coverage was increased by performing single-nucleotide polymorphism (SNP) imputation on the genome build GRCh38 using the Michigan Imputation Server (https://imputation.biodatacatalyst.nhlbi.nih.gov/index.html#!) and haplotypes generated by the Trans-Omics for Precision Medicine (TOPMed) program (freeze 5)⁶⁶, for cases and controls. We used the population panel “ALL” and filtered by an imputation of R²>0.1. Next, we only retained SNPs with R²≥0.6 and minor allele frequency (MAF)≥1%. Then, we checked cases and controls for solving within-Italian relationships and for testing the possible existence of population stratification within and across batches, by performing principal component analysis (PCA), using a LD-pruned subset of SNPs across chromosome 10 and the Plink v.1.9 package⁶⁷. The final set of analyzed variants comprised 3,425 SNPs, distributed in the MBL2 region (the gene +/−500 kb).

### MBL Plasma Concentration

Venous blood samples were collected during the first days after hospital admission [median (IQR): 3 (1-6) days], and EDTA plasma was stored at −80° C. MBL plasma concentrations were measured by ELISA (HycultBiotech, HK323-02, detection limit 0.41 ng/mL), by personnel blind to patients' characteristics. Measurements were taken from distinct samples tested in duplicate. In each analytical session, a sample from a pool of healthy donors plasma was used as internal control.

### Statistical Analysis

Prism GraphPad software v. 8.0 (www.graphpad.com) was used for the statistical analyses. Comparison among groups were performed using one or two-way analysis of variance (ANOVA) and the Bonferroni's correction. Non-linear fit of transformed data was determined by using the log (agonist) vs. response (three or four parameters). ROUT test or Rosner's test were applied to identify outliers. No outliers were identified and all data were included in the statistical analysis concerning FIGS. 6-10 and FIGS. 12, 13, 15. For FIG. 11c and d, 3 outliers were excluded, based on Rosner's test.

For genetic studies, case-control allele-dose association tests were performed using the PLINK v.1.9 logistic-regression framework for dosage data. Age, sex, age*age, sex*age, and the first 10 principal components from PCA were introduced in the model as covariates. Analyses were conducted always referring to the minor allele. All P values are presented as not corrected and accompanied by odds ratio (OR) and 95% confidence interval (CI); however, in the relevant table/figure, Bonferroni-corrected thresholds for significance are indicated in the footnote/legend.

In the genotypic analysis we evaluated the distribution of cases and controls carrying functional SNPs (rs5030737, rs1800450, rs1800451, and rs7096206)23.24.25.26 in biallelic conditions. Rs5030737, rs1800450, and rs1800451 are located in the coding region of the gene, and they are known to result in a severe impairment of the assembly of MBL trimeric structure. Alternative alleles of these SNPs are classically referred to as “D”, “B”, and “C”, but usually the presence of either of them is indicated as “allele 0”, whereas the wild-type allele is indicated as “allele A”. Rs7096206 is located in the promoter region, and it has been associated with modulation of MBL concentrations; the wild-type allele is classically indicated as “Y”, whereas the alternative one is called “X”. The statistical analysis in biallelic conditions was performed using a binomial glm model in R with the following covariates: age, sex, age*age, sex*age, and 10 principal components as already calculated for previous analyses.

To test the correlation between genetic variants and MBL concentrations, patients were stratified based on the genotypes of the rs10824845 and/or the presence of at least one allele 0 in one of the rs5030737, rs1800450, or rs1800451 genotypes. Outliers were identified using the Rosner's test and MBL plasma values>1.5* IQR were excluded from the statistical analysis. Box-Cox transformation was used to normalize data, before applying further statistical analyses. ANOVA and t-test were used to evaluate differences in MBL-transformed concentrations in different study groups.

Haplotype analysis was performed in two ways: i) by selecting relevant SNPs and using the -hap-logistic option implemented in PLINK v.1.07⁶⁸; ii) by an unsupervised approach by means of the Beagle software v3.3 and 5.1 (http://faculty.washington.edu/browning/beagle/b3.html), which uses the method described by Browning & Browning⁶⁹ for inferring haplotype phase. In this case, we used the default setting of 1,000 permutations for calculating corrected P values.

In the meta-analysis, we took advantage of association data deposited in the Regeneron -Genetic Center database (https://rgc-covid19.regeneron.com/home) for the GHS study (Geisinger Health System; data available for 869 cases and 112,862 controls of European ancestry). Pooled ORs and CIs were calculated using the Mantel-Haenszel model⁷⁰.

To test the role of rare variants in the susceptibility to a severe outcome, we analyzed data from Regeneron, focusing on the gene burden analysis in European cohorts considering the phenotype “COVID-19 positive hospitalized vs COVID-19 negative or COVID-19 status unknown”. In the database, variants are grouped in 4 categories based on their predicted effect at protein level, and their frequency in the population. Missense variants are classified according to the prediction made by 5 algorithms (SIFT, PolyPhen2 HDIV, PolyPhen2 HVAR, LRT, Mutation Taster)²⁸. We focused on the analysis of the most severe classes of variants: M1 (comprising only loss-of-function variants, LOF) and M3 (comprising LOF and all the missense variants predicted as damaging by the 5 aforementioned algorithms), and on rare (MAF<1%), as well as ultra-rare (singleton) variants.

No statistical methods were used to pre-determine sample sizes but our sample sizes are similar to those reported in previous publications^{12,65,71}. For experiments with cells, randomization was not necessary, since all groups derived from the same cell culture.

### Results

### Example 2

### Interaction of Humoral PRM With SARS-COV-2 Proteins

To study the role of humoral PRMs in recognizing SARS-COV-2, we first investigated the interaction between recombinant human humoral innate immunity molecules and SARS-CoV-2 proteins using a solid-phase binding assay. We first analyzed pentraxins and we did not observe specific binding of CRP or SAP to any of the SARS-COV-2 proteins tested (S1, S2, S protein active trimer, Nucleocapsid, Envelope protein) (FIGS. 6a and 6b). In contrast, PTX3 bound specifically and in a dose-dependent manner to the Nucleocapsid protein, one of the most abundant proteins of SARS-COV-217 (FIG. 6c). We validated this result by confirming the binding of PTX3 with SARS-COV-2 Nucleocapsid protein obtained from different sources (FIG. 12). PTX3 is a multimeric glycoprotein arranged in an octameric structure. Each protomer comprises a flexible N-terminal region and a C-terminal domain with homology to the short pentraxin family⁵. To define which portion of the molecule was involved in the interaction, we compared the binding of full-length PTX3 and its N-terminal or C-terminal domains to SARS-COV-2 Nucleocapsid protein. Results indicate that PTX3 interacts with SARS-COV-2 Nucleocapsid protein mainly through its N-terminal domain, although with lower affinity than full-length PTX3 (FIG. 6d).

We next investigated the interaction between PRMs of the classical and the lectin pathway of complement (C1q and the collectin MBL, respectively) and the viral proteins. C1q did not interact with any protein tested (FIG. 7a). In contrast, human MBL bound to SARS-COV-2 Spike protein (Wuhan strain1, active trimer), but not to the individual SARS-COV-2 Spike subunits S1 [containing the receptor-binding domain (RBD)] and S2 (containing the membrane fusion domain) (FIG. 7b). We validated these data by analyzing the binding of MBL to different recombinant SARS-COV-2 Spike proteins obtained from different sources or produced in-house either in HEK293 cells, CHO cells, or insect cells (FIG. 7c). All these preparations were bound by MBL, although with some differences. Notably, we did not observe binding when we tested a non-covalent trimer of the SARS-COV-2 Spike protein. These results indicate that a native-close structure of the SARS-COV-2 Spike protein (presumably in the trimeric conformation) is indispensable for MBL recognition.

MBL is a member of the collectin family, a class of PRMs composed of a Ca²⁺-type lectin domain (also called Carbohydrate Recognition Domain, CRD) and a collagen-like domain⁶. Thus, we analyzed the interaction of SARS-COV-2 Spike protein with other collectins involved in innate immunity, such as Collectin-10, Collectin-11 and Collectin-12 (also known as CL-L1/CL-10, CL-K1/CL-11 and CL-P1/CL-12) and the pulmonary surfactant proteins SP-A and SP-D. We also extended the analysis to recombinant Ficolin-1, -2, or -3, a family of proteins known to activate the complement lectin pathway, and structurally-related to MBL. In contrast with MBL, CL-L1, CL-K1, CL-P1, SP-A, SP-D, and ficolins did not bind to SARS-COV-2 Spike protein (FIG. 7d,e and FIG. 12), indicating that recognition of Spike is unique to MBL.

We further characterized the interaction of SARS-COV-2 Spike protein with MBL by Surface Plasmon Resonance (SPR). Different concentrations of recombinant, SARS-COV-2 Spike protein or RBD domain were flowed onto MBL immobilized on the biosensor surface. Trimeric SARS-COV-2 Spike protein formed a stable calcium-dependent complex with nanomolar affinity (K_{D}=34 nM) whereas MBL did not bind the isolated RBD (FIG. 7f and FIG. 13), confirming the results obtained using the S1 subunit.

These results indicate that out of 12 humoral PRM tested in a solid-phase binding assay, PTX3 and MBL bound the SARS-COV-2 Nucleoprotein and Spike, respectively.

### Example 3

### Interaction of MBL With Spike Pseudotyped Lentivirus

To mimic the interaction between MBL and SARS-COV-2 Spike protein in its physiological conformation in the viral envelope, we investigated the binding of viral particles of SARS-COV-2 Spike protein pseudotyped on a lentivirus vector to MBL-coated plates. The interaction was determined by lysing the bound pseudovirus and measuring the released lentiviral vector p24 core protein by ELISA. While lentiviral control particles pseudotyped with the VSV-g glycoprotein (VSV-pseudovirus) did not result in any binding, those exposing the SARS-COV-2 Spike protein showed specific interaction with MBL (FIG. 8a). These data strongly suggest that MBL can also interact with the SARS-COV-2 Spike protein exposed on the virus surface.

### Example 4

### MBL Interacts With Glycosidic Sites of the SARS-COV-2 Spike

The SARS-COV-2 Spike protein is highly glycosylated, as recently described¹⁸. Out of the 22 N-glycosylation sites, 8 contain oligomannose-types glycans, which could be interaction sites for the MBL carbohydrate recognition domain. To address this possibility, we performed a solution-based competition assay with D-mannose and N-acetyl-glucosamine, two specific ligands of the lectin. D-mannose and N-acetyl-glucosamine inhibited MBL binding to the Spike protein (FIG. 8b), thus confirming the Ca²⁺-dependent interaction between the MBL lectin domain and the glycosidic sites exposed by the Spike protein. D-Glucose, a non-specific ligand of MBL, inhibited the interaction only at higher concentrations (FIG. 8b). Based on the alignment of MBL crystal structure with mannose molecules (FIG. 8c), we identified 14 putative binding sites on the Spike protein (FIG. 8d). Next, we considered sites having a high (>80%) oligomannosylation occupancy¹⁸. This analysis provided two possible MBL binding sites, namely N603, N801 and N1074 all on the same Spike chain, or N603, N1074 and N709 with N709 on a neighboring chain (FIG. 8e). Interestingly, in both cases, the hypothesized MBL binding sites span across the S1 and S2 region of the Spike protein (FIG. 8f), providing hints to a possible inhibition mechanism. These data indicate that the glycosylation state of the SARS-COV-2 Spike protein is important for its interaction with MBL.

### Example 5

### Interaction of MBL With Spike From VoC

We then tested whether MBL recognized Spike proteins from VoC. First, we analyzed whether the reported mutations affected the known 22 glycosylation sites of each protomer. FIG. 8g shows a schematic representation of the 22 positions of N-linked glycosylation sequons and of 66 known mutations of VoC and VoI, including the recently added ε, λ and o (Omicron) variants, indicating that none of these mutations involve the glycosylation sites, and suggesting that MBL could interact with the variants with the same affinity. In agreement with our binding assays, no MBL target sites are expected in the RBD. Interestingly, the predicted MBL binding sites are conserved in the Omicron VoC, as shown in the Spike-MBL complex model (FIG. 14). We assessed by solid phase assay the interaction of MBL with the SARS-COV-2 D614G Spike trimeric protein, the B.1.1.7 variant (α, emerged in UK), the B.1.1.28 or P.1 variant (γ, emerged in Brazil), the B.1.351 variant (β, emerged in South Africa), and with the B.1.617.2 variant (δ, emerged in India), which is currently a major concern worldwide (FIG. 8h). In agreement with the in silico analysis, MBL bound efficiently the VoC Spike proteins tested. These results indicate that the potential MBL binding sites are conserved in the VoC Spike proteins and the affinity of the interaction with MBL is not affected by these mutations.

### Example 6

### Complement Lectin Pathway Activation

Next, we tested whether the interaction of MBL with Spike could activate the complement lectin pathway. We incubated SARS-COV-2 Spike protein-coated plates with human serum, or C1q- or C4- or C3-depleted serum, and we assessed the deposition of C5b-9. Incubation with either normal human serum or C1q-depleted serum resulted in complement deposition mediated by SARS-COV-2 Spike protein (FIG. 8i, left). Conversely, incubation with a serum depleted of

C4 strongly reduced C5b-9 deposition, with levels comparable to those observed with heat-inactivated serum or C3-depleted serum. Reconstitution of C4-depleted serum with purified C4 restored C5b-9 deposition levels similar to those observed with normal human serum. To further address the role of MBL in SARS-COV-2 Spike protein-mediated complement activation, we assessed C5b-9 deposition by incubating normal human serum or MBL-immunodepleted serum over captured SARS-COV-2 Spike protein, either as active, or non-covalent trimer (FIG. 8i, right). In agreement with the binding data, no complement deposition was observed with the non-covalent trimeric Spike protein. Notably, immunodepletion of MBL from human serum resulted in a significant reduction in C5b-9 deposition, which could be fully reverted by the addition of rhMBL (FIG. 8i, right). These data clearly indicate that SARS-COV-2 Spike, by interacting with MBL, activates the complement lectin pathway.

### Example 7

### SARS-COV-2 Inhibition by MBL

To validate the relevance of the interaction between MBL and SARS-COV-2 Spike protein, we investigate whether MBL inhibited SARS-COV-2 entry in susceptible cells. We first tested the effect of MBL and other soluble PRMs (10-fold serial dilution, from 0.01 to 10 μg/ml) on the entry of the viral particles of SARS-COV-2 Spike protein pseudotyped on a lentivirus vector in 293T cells overexpressing Angiotensin-Converting Enzyme 2 (ACE2). Among the soluble PRMs tested, MBL was found to be the only molecule with anti-SARS-COV-2 activity. Spike-mediated viral entry was inhibited by 90% at the highest concentration of 10 μg/ml (34 nM) with an EC50 value of approximately 0.5 μg/ml (1.7 nM) (FIG. 9a). As control, entry of lentiviral particles pseudotyped with the VSV-g glycoprotein was not inhibited by MBL (FIG. 9a).

We next tested the antiviral activity of MBL on the SARS-COV-2 infection of lung epithelial models relevant to human infections. Among a number of lung-derived epithelial cell lines, Calu-3 (human lung adenocarcinoma) cells have been shown to be permissive to SARS-CoV-2 infection19. SARS-COV-2 (D614G variant, MOI=0.1 and 1) was preincubated in complete medium containing different concentrations of MBL (0.01-10 μg/mL; 0.034-34 nM) before incubation with Calu-3 cells. After 48 and 72 h, the infectivity of SARS-COV-2 present in cell culture supernatants was determined by a plaque-forming assay in monkey-derived Vero cells. Vero cells are a handy cell line used worldwide as it is devoid of the interferon (IFN) response²⁰ and, for this reason, highly supportive of virus replication. MBL showed a concentration-dependent inhibition of SARS-COV-2 infection of Calu-3 cells at MOI 0.1 and 1 (FIG. 15a,), that was statistically significant at 1 and 10 μg/ml (3.4 and 34 nM) 72 h after infection. When both virus and cells were pre-incubated with the same concentrations of MBL (0.01-10 μg/mL; 0.034-34 nM), the antiviral activity increased significantly from 0.1 μg/ml (0.34 nM) to the top concentration of 10 μg/ml (34 nM), 72 h post-infection (PI) (FIG. 9b and FIG. 15b). The calculated EC50 was 0.08 μg/mL (0.27 nM) at 72 h PI. Notably, MBL showed a concentration-dependent inhibition of infection of Calu-3 cells also by SARS-COV-2 B.1.1.7 (α) variant at MOI 0.1 (FIG. 9c) and MOI 0.01 (FIG. 15c), as well as by B.1.351 (β) variant at MOI 0.1 (FIG. 9d), P.1 (γ) variant at MOI 0.1 (FIGS. 9e), and B.1.617.2 (δ) variant at MOI 0.1 (FIG. 9f).

Furthermore, a 3D-human bronchial epithelial cells (HBEC) model was used to test whether MBL inhibited SARS-COV-2 replication. SARS-COV-2 production at the epithelial apical surface increased sharply at 48 h PI (not shown), reaching 48×10⁶±6×10⁶ (mean±SEM) PFU/ml 72 h PI. Treatment of HBEC with MBL decreased viral production to 4×10⁶+0.8×10⁶ PFU/ml 72 h PI at the highest concentration of 50 μg/ml (170 nM) (FIG. 10a). In contrast, PTX3 treatment was ineffective at inhibiting virus production (FIG. 10d). We then assessed whether MBL affected inflammatory responses in HBEC upon SARS-COV-2 infection under these experimental conditions. MBL treatment inhibited the production of interleukin-8 (IL-8) and CXCL5, two chemokines involved in myeloid cell recruitment and activation (FIG. 15e).

We finally evaluated the occurrence of MBL-Spike protein interaction in SARS-COV-2-infected HBEC by confocal microscopy. MBL colocalized with SARS-COV-2 Spike protein in infected cells (FIG. 10b,c). In 3D rendered images of the HBEC cell cultures (FIG. 10d), colocalization was preferentially associated with the apical side of cytokeratin 14 positive cells. Evidence of the interaction between MBL and SARS-COV-2 Spike protein in infected HBEC at molecular scale (<100nm XY spatial resolution) were also obtained in STED-based super-resolution microscopy (FIG. 10e).

These results indicate that MBL inhibits SARS-COV-2 infection of a human lung-derived epithelial cell line and primary bronchial cells, reduces the induced inflammatory response, and colocalizes with SARS-COV-2 Spike protein in infected cells.

### Example 8

### MBL2 Variants and Haplotypes are Associated With Severe COVID-19

### MBL2 Variants are Associated With Severe COVID-19

Human MBL is encoded by the MBL2 gene, which contains polymorphic variants both in the regulatory and structural part of the gene. These variants are associated with the serum concentration of the protein²¹. MBL2 genetic variants have been shown to correlate with increased susceptibility to selected infections, including SARS²². To explore the significance of our in vitro results in the frame of the COVID-19 pandemic, we investigated the possible association of MBL2 polymorphisms with severe COVID-19 with respiratory failure in an Italian cohort of 332 cases and 1,668 controls (general population). We initially focused on six SNPs known to be associated with MBL protein levels (Table 2)^{23,24,25,26}. We observed a significant difference only in the frequency of the rs5030737-A allele between patients and controls (7.7% and 6.0%, respectively; OR=1.43, 95% CI=1.00-2.05, P=0.049; Table 2), which however did not survive the correction for multiple testing. We also verified the distribution of cases and controls carrying these functional SNPs in biallelic conditions, by specifically focusing on the three missense variants and on the promoter SNP known to confer the strongest effect on MBL2 expression (rs7096206). In agreement with in vitro functional assays, a significant predisposing effect was observed in those individuals carrying two disruptive alleles among rs5030737, rs1800450, and rs1800451 (OR=2.09, 95% CI=1.18-3.71, P=0.011; Table 4).

When we compared the frequencies of haplotypes determined by all six SNPs, we found the CCGGCC haplotype frequency significantly decreased in patients with severe COVID-19 (26.7% in cases, 30.4% in controls). This haplotype shows a protective effect (OR=0.78, 95% CI=0.65-0.95, P=0.025; Table 5), consistently with the lack of the rs5030737-A allele, which is only present in the CCAGCC haplotype (OR=1.38, 95% CI=1.00-1.90; P=0.078; Table 5).

Though borderline, these first association results encouraged us to investigate the 1-Mb-long genomic region encompassing the MBL2 gene systematically. To this aim, we performed single-SNP as well as haplotype-based association analyses using genotyped/imputed data on 3,425 polymorphisms. Single-SNP association analysis revealed three suggestive signals (rs150342746, OR=3.47, 95% CI=1.81-6.68, P=1.86*10-+; rs10824845, OR=1.76, 95% CI=1.30-2.39, P=2.91*10⁻⁴; and rs11816263, OR=1.42, 95% CI=1.17-1.73, P=3.47*10⁻⁴; Table 3; FIG. 11a, b), whereas haplotype-based analysis disclosed 7 haplotypes of different lengths, from 2 to 24 SNPs, strongly associated with severe COVID-19 (all surviving the correction for multiple tests; FIG. 11; Table 6). Among them, the one composed of polymorphisms rs10824844-rs10824845 incorporates one of the two top-markers evidenced by the single-SNP association analysis and is present in 12.2% of cases and 6.9% of controls (TA haplotype, OR=1.88, 95% CI=1.44-2.45, P=1.04*10⁻⁵; Table 6). Hence, we performed a meta-analysis based on the rs10824845 polymorphism by including the GHS study of COVID-19 patients: this resulted in a pooled OR=1.32, 95% CI=1.15-1.52, P=9.12*10⁻⁵ (Table 7). Notably, the rs10824845 polymorphism points to a regulatory region characterized by the presence of an enhancer (GH10J052964), described as a distant modulator of MBL2 gene expression. This regulatory element is active in HepG2 cells (hepatocytes), as well as MO (from venous blood) and MI (from cord and venous blood) macrophages (data from the GeneHancer database²⁷, available through http://www.genecards.org/).

We also interrogated the Regeneron database²⁸ to analyze the role of rare genetic variants in the MBL2 gene in the predisposition to severe COVID-19. We depict the burden analyses both on singletons and on rare damaging variants with minor allele frequency (MAF)<1% (Table 8). The meta-analysis was focused on the European population and evidenced the significant contribution of singleton variants. This was observed when only loss-of-function variants were considered (M1 analysis, OR=32.05, 95% CI=2.27-452.7, P=0.010; Table 8) and when both loss-of-function and missense variants, predicted as damaging by 5 algorithms, were analyzed (M3 analysis, OR=23.6, 95% CI=3.44-162.09, P=0.0013; Table 8).

Moreover, the same database reports a significant association for the rs35668665 polymorphism both with susceptibility to COVID-19 (OR=4.11, GHS cohort) and with severity of symptoms (OR=7.91, UK BioBank cohort). Interestingly, this variant maps in correspondence of the last nucleotide of MBL2 exon 1, thus possibly interfering with the splicing process.

Finally, we measured MBL plasma concentrations at hospital admission in 40 patients from the Humanitas Clinical and Research Center cohort and correlated them to MBL2 genetic variants. We first focused on the three missense variants (functional SNPs rs5030737, rs1800450, rs1800451) and grouped individuals carrying at least one alternative allele (allele 0) compared to those carrying the wild-type allele. We observed a significantly lower MBL plasma concentration (P=6.2*10⁻⁸) in individuals carrying at least one alternative allele (allele 0) compared to those carrying the wild-type allele (FIG. 11c). Then we analyzed the impact of the rs10824845 SNP, by stratifying the same patients according to their genotype. We observed a reduction of MBL concentration in heterozygous individuals, though not significant (1.2-fold decrease in heterozygous; P=0.11). However, when considering the contribution of A/0 alleles, a clear genotype-dependent modulation of MBL concentrations emerged (P=1.1*10⁻⁶; FIG. 11d). Altogether, these analyses indicate that MBL2 genetic variants are associated with COVID-19 severity and impact on protein abundance of this “ante-antibody”.

TABLE 2
Association analysis results: candidate SNP association analysis

| | | | A1/A2 | MAF | MAF | | 95% | | Direction |
|---|---|---|---|---|---|---|---|---|---|
| SNP | rsID | Variation | (legacy) | cases | controls | OR | CI | P* | (A1) ** |
| chr10: 52771466: C: T | rs1800451 | p.Gly57Glu | T/C | 0.017 | 0.021 | 0.814 | 0.39-1.72 | 0.588 | Lowers MBL |
| | | | (C/A) | | | | | | levels |
| chr10: 52771475: C: T | rs1800450 | p.Gly54Asp | T/C | 0.017 | 0.15 | 1.070 | 0.83-1.39 | 0.609 | Lowers MBL |
| | | | (B/A) | | | | | | levels |
| chr10: 52771482: G: A | rs5030737 | p.Arg52Cys | A/G | 0.077 | 0.06 | 1.434 | 1.00-2.05 | 0.049 | Lowers MBL |
| | | | (D/A) | | | | | | levels |
| chr10: 52771701: G: A | rs7095891 | Promoter | A/G | 0.221 | 0.243 | 0.827 | 0.66-1.04 | 0.104 | — |
| | | region | | | | | | | |
| chr10: 52771925: G: C | rs7096206 | Promoter | G/C | 0.215 | 0.205 | 1.201 | 0.96-1.51 | 0.113 | Lowers MBL |
| | | eQTL in | (X/Y) | | | | | | levels |
| | | liver | | | | | | | |
| | | (P = 1.7*10⁻¹⁷) | | | | | | | |
| chr10: 52772254: G: C | rs11003125 | Promoter | C/G | 0.342 | 0.364 | 0.886 | 0.73-1.09 | 0.236 | Increases |
| | | eQTL in | | | | | | | MBL levels |
| | | liver | | | | | | | |
| | | (P = 9.1*10⁻⁶) | | | | | | | |

The SNP column is in the format chromosome: position: reference allele: alternative allele. The position refers to hg38 version of the genome.
A1/A2 refers to minor/major alleles; legacy names refer to allele names as indicated in the literature (see text for relevant references); A: wild-type allele; B, C, and D: alternative alleles, collectively called “allele 0”. Y, X: wild-type and alternative alleles of the rs7096206 polymorphism.
A1 = minor allele; A2 = major allele; CI = confidence interval; MAF = minor allele frequency; OR = odds ratio; rsID = reference sequence identification number; SNP = single nucleotide polymorphism.
*Bonferroni threshold for significance is P < 0.008.
** Direction derived from either the literature or the GTEx database (The Genotype-Tissue Expression database; https://www.gtexportal.org/home/).

TABLE 3
Locus-wide association analysis

| SNP | rsID | A1 | MAF cases | MAF controls | OR | 95% CI | P* |
|---|---|---|---|---|---|---|---|
| chr10: 53229424: C: T | rs150342746 | T | 0.026 | 0.008 | 3.474 | 1.808-6.676 | 1.86*10⁻⁴ |
| chr10: 52963964: G: A | rs10824845 | A | 0.124 | 0.072 | 1.762 | 1.297-2.393 | 2.91*10⁻⁴ |
| chr10: 53083059: C: A | rs11816263 | A | 0.386 | 0.315 | 1.422 | 1.173-1.725 | 3.47*10⁻⁴ |
| chr10: 53104393: A: G | rs74974397 | G | 0.071 | 0.041 | 1.813 | 1.235-2.661 | 0.0024 |
| chr10: 53082503: A: AT | rs71032688 | A | 0.258 | 0.191 | 1.415 | 1.128-1.776 | 0.0025 |
| chr10: 53155596: C: T | rs117108247 | T | 0.069 | 0.042 | 1.750 | 1.195-2.561 | 0.0040 |

The SNP column is in the format chromosome: position: reference allele: alternative allele. The position refers to hg38 version of the genome.
SNPs with P < 0.0050 are shown.
SNP = single nucleotide polymorphism; rsID = reference sequence identification number; A1 = minor allele; MAF = minor allele frequency; OR = odds ratio; CI = confidence interval.
*Bonferroni threshold for significance is P < 1.5*10⁻⁵.

TABLE 4
Association analysis results: Biallelic variant analysis

| Genotypes* | OR | 95% CI | P |
|---|---|---|---|
| X0/X0, Y0/Y0, X0/Y0 | 2.093 | 1.18-3.71 | 0.011 |
| XA/Y0, YA/X0 | 1.123 | 0.65-1.93 | 0.674 |
| XA/XA | 1.251 | 0.59-2.64 | 0.558 |
| XA/YA | 1.448 | 0.97-2.16 | 0.070 |
| YA/Y0 | 1.209 | 0.83-1.77 | 0.326 |

*Genotypes are indicated as legacy names which refer to allele names as indicated in the literature (see text for relevant references); A: wild-type allele; B, C, and D: alternative alleles of the rs1800450, rs1800451 and rs5030737, respectively, collectively called “allele 0”. Y, X: wild-type and alternative alleles of the rs7096206 polymorphism. The analysis is referred to the wild-type genotype: YA/YA. The statistical analysis was performed using a binomial glm model.
OR = odds ratio; CI = confidence interval.

TABLE 5
Haplotype analysis for candidate SNPs
(rs1800451|rs1800450|rs5030737|rs7095891|rs7096206|rs11003125)

| | Frequency | Frequency | | | |
|---|---|---|---|---|---|
| Haplotype | in cases | in controls | OR | CI | P* |
| CCAGCC | 0.075 | 0.060 | 1.380 | 1.000-1.903 | 0.078 |
| CCGGCC | 0.267 | 0.304 | 0.785 | 0.651-0.946 | 0.025 |
| CCGGGG | 0.216 | 0.205 | 1.190 | 0.971-1.458 | 0.130 |
| TCGACG | 0.017 | 0.0210 | 0.870 | 0.458-1.652 | 0.704 |
| CCGACG | 0.205 | 0.222 | 0.833 | 0.678-1.023 | 0.121 |
| CTGGCG | 0.166 | 0.150 | 1.090 | 0.870-1.366 | 0.501 |
| CCGGCG | 0.054 | 0.038 | 1.500 | 1.026-2.192 | 0.056 |

*Bonferroni threshold for significance is P < 0.05.
OR = odds ratio; CI = confidence interval.

TABLE 6
Locus-wide haplotype analysis

| | | Frequency | | | | | |
|---|---|---|---|---|---|---|---|
| | Frequency | in | | | | P | |
| Haplotype | in cases | controls | OR | CI | P | permutation* | SNPs** |
| ATCGCAA | 0.006 | 0.043 | 0.133 | 0.049-0.36 | 2.26*10⁻⁷ | 9.99*10⁻⁴ | 6 SNPs, |
| | | | | | | | rs11344513\|rs7071467 |
| CCC | 0.005 | 0.073 | 0.058 | 0.019-0.182 | 3.12*10⁻¹⁶ | 9.99*10⁻⁴ | 3 SNPs, |
| | | | | | | | rs17662822\|rs1159798\| |
| | | | | | | | rs1912619 |
| TCCCC | 0.000 | 0.021 | <1.00 | nc | 1.17*10⁻⁵ | 0.019 | 5 SNPs, |
| | | | | | | | rs2204344\|rs12218074\| |
| | | | | | | | rs80035245\|rs7935712\| |
| | | | | | | | rs10824836 |
| TCAGACC | 0.032 | 0.007 | 4.92 | 2.69-9 | 2.59*10⁻⁶ | 4.99*10⁻³ | 5 SNPs, |
| | | | | | | | rs16935439\|rs147096903\| |
| | | | | | | | rs10824839\|rs11003267\| |
| | | | | | | | rs11003268 |
| TA | 0.122 | 0.069 | 1.876 | 1.435-2.453 | 1.04*10⁻⁵ | 0.018 | 2 SNPs, |
| | | | | | | | rs10824844\|rs10824845 |
| ATCCCCG | 0.000 | 0.040 | <1.00 | nc | 3.41*10⁻⁷ | 9.99*10⁻⁴ | 9 SNPs, rs57504125\| |
| CATTGA | | | | | | | chr10: 5308418:G:A |
| [SEQ ID | | | | | | | |
| N. 3] | | | | | | | |
| AGATCCC | 0.237 | 0.170 | 1.509 | 1.235-1.844 | 3.28*10−6 | 4.99*10⁻³ | 24 SNPs, |
| CGCGCGT | | | | | | | rs71032688\|rs7092597 |
| GCAACGG | | | | | | | |
| CTGCGGA | | | | | | | |
| [SEQ ID | | | | | | | |
| N. 4] | | | | | | | |

P values were calculated using Fisher's exact test.
*P Value permutation as calculated after performing 1,000 permutations to correct for multiple testing.
**The number of SNPs composing the haplotype is indicated. All the SNPs forming the haplotype are shown for short
haplotypes (including max 5 SNPs). For more complex haplotypes (including > 5 SNPs) only the first and the last
SNPs are indicated. OR = odds ratio; CI = confidence intercal; nc = not calculated.

TABLE 7
Meta-analysis for the rs10824845 polymorphism

| | Cases | Controls | | | |
|---|---|---|---|---|---|
| | (A1/A2 | (A1/A2 | | | |
| Cohort | alleles) | alleles) | OR | 95% CI | P |
| Italian | 82/582 | 241/3,095 | 1.81 | 1.392-2.353 | 9.57*10⁻⁶ |
| cohort | | | | | |
| GHS | 180/1,558 | 18,979/205,845 | 1.20 | 1.024-1.396 | 0.023 |
| cohort | | | | | |
| Summary | | | 1.32 | 1.149-1.520 | 9.12*10⁻⁵ |

A1 = minor allele; A2 = major allele; CI = confidence interval; OR = odds ratio.
P values, pooled ORs and CIs were calculated using the Mantel-Haenszel model.

TABLE 8
Rare variants analysis

| | Variant | | Cases | Controls | | | |
|---|---|---|---|---|---|---|---|
| Category* | frequency | Studies** | (A1A1:A1A2:A2A2) | (A1A1:A1A2:A2A2) | OR | 95% CI | P |
| M1 | singleton | GHS, UKB | 1872:1:0 | 508520:12:0 | 32.05 | 2.27-452.7 | 0.01027 |
| M1 | MAF < 1% | GHS, UKB | 1872:1:0 | 508475:57:0 | 9.91 | 1.07-91.77 | 0.04343 |
| M3 | singleton | GHS, UKB | 1871:2:0 | 508501:31:0 | 23.6 | 3.44-162.09 | 0.0013 |
| M3 | MAF < 1% | GHS, UKB | 1871:2:0 | 508242:290:0 | 2.64 | 0.38-18.54 | 0.3292 |

P values were calculated using a genome-wide Firth logistic regression test²⁸.
*M1: comprises loss-of-function variants; M3: comprises loss-of-function and missense variants predicted as damaging by 5 algorithms.
**GHS: Geisinger Health System; UKB: UK Biobank.
A1 = minor allele; A2 = major allele; CI = confidence interval; OR = odds ratio; MAF = minor allele frequency.

TABLE 9
Recombinant SARS-CoV-2 proteins used in this study.

| Proteins | Host | Cat | Company |
|---|---|---|---|
| SARS-CoV-2 S1 protein, His Tag | HEK293 | S1N-C52H4 | ACROBiosystems |
| SARS-CoV-2 S2 protein, His Tag | HEK293 | S2N-C52H5 | ACROBiosystems |
| SARS-CoV-2 S protein, His Tag, | HEK293 | SPN-C52H8 | ACROBiosystems |
| active trimer | | | |
| SARS-CoV-2 Nucleocapsid protein, | HEK293 | NUN-C5227 | ACROBiosystems |
| His Tag | | | |
| SARS-CoV-2 Envelope protein. GST, | E. coli | ENN-C5128 | ACROBiosystems |
| His Tag | | | |
| Biotinylated SARS-CoV-2 S protein, | HEK293 | SPN-C82E3 | ACROBiosystems |
| His Tag, active trimer | | | |
| Biotinlyated SARS-CoV-2 Nucleocapsid | HEK293 | NUN-C82E8 | ACROBiosystems |
| protein, His Tag | | | |
| SARS-CoV-2 S protein, His Tag | HEK293 | 10549-CV | R&D Systems |
| SARS-CoV-2 Nucleocapsid protein, | HEK293 | 230-30164 | RayBiotech |
| His Tag | | | |
| SARS-CoV-2 S protein, His Tag | HEK293 | In house | ⁴⁰ |
| SARS-CoV-2 S protein trimer, | EXPI293F | In house | ⁵³ |
| His Tag | cells | | |
| SARS-CoV-2 S protein, His Tag | CHO | XLGCOV-1-PPTH | ExcellGene |
| SARS-CoV-2 (2019-nCoV) Spike RBD, | HEK293 | 40592-V08H | Sino Biological |
| His Tag | | | |
| SARS-CoV-2 (2019-nCoV) Spike S1 + S2 | Insect cells | 40589-V08B1 | Sino Biological |
| ECD, His Tag | | | |
| SARS-CoV-2 (2019-nCoV) Spike S1 + S2 | Insect cells | 40589-V08B6 | Sino Biological |
| ECD (B.1.1.7), His Tag | | | |
| SARS-CoV-2 S protein (D614G), His Tag | HEK293 | SPN-C52H3 | ACROBiosystems |
| SARS-CoV-2 S protein (B.1.1.7 variant), | HEK293 | SPN-C52H6 | ACROBiosystems |
| His Tag | | | |
| SARS-CoV-2 S protein (B.1.351 variant), | HEK293 | SPN-C52Hc | ACROBiosystems |
| His Tag | | | |
| SARS-CoV-2 S protein (B.1.1.28 variant), | HEK293 | SPN-C52Hg | ACROBiosystems |
| His Tag | | | |
| SARS-CoV-2 S protein (B.1.617.2 variant), | HEK293 | SPN-C52He | ACROBiosystems |
| His Tag | | | |

### Discussion

Among the 12 fluid phase PRM tested in this study, only PTX3 and MBL bound SARS-CoV-2 virus components. PTX3 recognized the viral Nucleoprotein and had no antiviral activity. PTX3 was expressed at high levels by myeloid cells in blood and lungs and its plasma concentrations have strong and independent prognostic significance for death in COVID-19 patients^{16,29}. It remains to be elucidated whether PTX3 plays a role in Nucleocapsid-mediated complement activation and cytokine production^{30,31,32}.

MBL recognized the SARS-COV-2 Spike protein, including that of four VoC, and had antiviral activity in vitro against all of them, including the B.1.617.2 variant (δ), which is currently a major concern worldwide. MBL has previously been shown to bind SARS-COV Spike³³. The interaction of MBL with SARS-COV-2 Spike required a trimeric conformation of the viral protein, did not involve direct recognition of the RBD, and was glycan-dependent, as expected. Site-specific glycosylation analysis of the SARS-COV-2 Spike protein revealed the presence of various oligomannose-type glycans across the protein¹⁸.

Molecular modelling reported here suggests that the MBL trimer interacts with glycans attached to the residues N603, N801 and N1074 on the same chain or N603, N709 and N1074 with N709 on a different chain. In both cases the hypothesized MBL binding site spans across the S1 and S2 region of SARS-COV-2 Spike, suggesting a possible neutralization mechanism. The binding of MBL could prevent the detachment of the SI region and the release of the fusion peptide at position 815, thus inhibiting virus entry into host cells. However, the mechanisms responsible for the antiviral activity of MBL remain to be fully defined. It is noteworthy that C-type lectins have been reported to act as entry receptors (or coreceptors)^{34,35,36} and MBL is likely to compete at this level.

In apparent contrast with our results, Ficolin-2 and Collectin-11 were recently shown to interact with S- and N-proteins, MBL with N-protein, and SP-D with S-protein^{37,38}. Experimental approaches used in these studies may explain the discrepancy with our results: whereas commercially available and in house produced recombinant pentraxins, C1q, MBL, ficolins, surfactant proteins, and collectins were used in our study, serum was used as source of PRMs by others³⁷, which may result in indirect interaction of MBL, Ficolin-2 or Collectin-11 with viral proteins mediated by a serum component. For instance, MASP-2 was shown to interact with N-protein³⁷, confirming a previous study³⁰. MASPs are normally present in plasma complexed with molecules of the lectin pathway, thus explaining the interactions of MBL with N-protein, which was not observed in our study. Concerning SP-D, Hsieh et al. observed an interaction between a recombinant fragment of SP-D and S-protein, whereas the recombinant full-length molecule showed a very low affinity for S-protein³⁸. To strengthen our results, we repeated the binding experiments using 4 different preparations of recombinant Ficolin-2, 2 of SP-D and 2 of Collectin-11 (as single molecule or as Collectin-10/11 heterocomplexes)³⁹, and we did not observe interaction with viral proteins. The studies by Ali et al.³⁷ and Hsieh et al. ³⁸ have the merit to underline the involvement of the lectin pathway in SARS-COV-2-dependent complement activation. Here, we provide a rigorous, solid, reliable, and comprehensive picture of recognition of SARS-COV-2 components by “ante-antibodies”.

Interestingly, the in silico analysis presented here indicates that mutations in variants reported until now, including Omicron, do not affect glycosylation sites containing oligomannose-types glycans potentially recognized by MBL. In addition, binding and infection experiments show that the anti-viral activity of MBL is not affected by these mutations. This finding indicates that the glycosylation sites are generally spared by selective pressure, suggesting they are essential for SARS-COV-2 infectivity. It has been recently shown that mechanisms of in vitro escape of SARS-COV-2 from a highly neutralizing COVID-19 convalescent plasma include the insertion of a new glycan sequon in the N-terminal domain of the Spike protein, which leads to complete resistance to neutralization⁴⁰. This result further emphasizes the relevance of Spike glycosidic moieties targeted by MBL in SARS-COV-2 infectivity.

MBL was found to interact with Spike and have antiviral activity with an EC50 of approximately 0.08 mg/ml (0.27 nM) and an affinity of 34 nM. These concentrations are well in the range of those found in the blood of normal individuals (up to 10 mg/ml), which increase 2-3-fold during the acute phase response. MBL plasma concentrations in healthy individuals are extremely variable, in part depending on genetic variation in the MBL2 gene²¹. Defective MBL production has been associated with an increased risk of infections, in particular in primary or secondary immunodeficient children⁴¹. In SARS, conflicting results have been reported concerning the relevance of MBL2 genetic variants in this condition^{22,42,43}. In COVID-19, one MBL2 polymorphism has been associated with the development and severity of the infection⁴⁴. We investigated the possible role of MBL2 genetic variants in determining susceptibility to severe COVID-19 with respiratory failure. Surprisingly and in contrast with a previous study⁴⁴, we found only a borderline correlation between one haplotype of the 6 SNPs associated with MBL levels and frequency of severe COVID-19 cases. However, we found a significant predisposing effect in individuals carrying MBL2 biallelic functional variants, as well as a total of 7 significantly associated haplotypes, distributed along the MBL2 genomic region, often mapping in correspondence of regulatory elements (such as enhancers, promoter region, histone marks). Our association data are reinforced by the meta-analysis results, obtained by integrating the summary statistics from a European cohort of >113,000 individuals, and by the fact that one of our second best associations (rs10824845) maps in proximity of a cluster of suggestive signals identified by the COVID-19 Host Genetic Initiative (https://www.covid19hg.org/), which includes data from up to 33 different worldwide studies. Further, the Regeneron—Genetic Center database²⁸ reports significant associations on rare and ultra-rare variants analyses. Finally, the rs5030737 (p.Arg52Cys) polymorphism in MBL2 has been described in the UKBiobank ICD PheWeb database (https://pheweb.org/UKB-SAIGE/) as a top signal in determining both “dependence on respirator [Ventilator] or supplemental oxygen” (ICD code Z99.1; P=2.7*10⁻⁴) and “Respiratory failure, insufficiency, arrest” (ICD code J96; P=2.7*10⁻³). These observations suggest that genetic variations in MBL2, possibly involved in the modulation of the expression of the gene in hepatocytes, and, interestingly, in macrophages, could play a role in determining susceptibility to severe COVID-19 with respiratory failure. Therefore, genetic analysis is consistent with the view that MBL recognition of SARS-COV-2 plays an important role in COVID-19 pathogenesis.

Upon interaction with Spike, MBL was found to activate the lectin pathway of complement, as expected. Complement has been credited an important role in the hyperinflammation underlying severe disease and is considered a relevant therapeutic target^{45,46}. Therefore, as for innate immunity in general including the IFN pathway⁴⁷, MBL-mediated recognition of SARS-COV-2 may act as a double-edged sword. In early phases of the disease MBL may serve as a mechanism of antiviral resistance by blocking viral entry, whereas in advanced disease stages it may contribute to complement activation and uncontrolled inflammation.

MBL has been safely administered to patients with cystic fibrosis and chronic lung infections in which MBL deficiency contributes to pathogenesis^{48,49}. Therefore, the results presented here have translational implications both in terms of comprehensive genetic risk assessment and development of local or systemic therapeutic approaches.

### REFERENCES

1. Wu, F. et al. A new coronavirus associated with human respiratory disease in China. Nature 579, 265-269 (2020).
2. Zhu, N. et al. A Novel Coronavirus from Patients with Pneumonia in China, 2019. N Engl J Med 382, 727-733 (2020).
3. Hartenian, E. et al. The molecular virology of coronaviruses. J Biol Chem 295, 12910-12934 (2020).
4. Wang, J., Jiang, M., Chen, X. & Montaner, L. J. Cytokine storm and leukocyte changes in mild versus severe SARS-COV-2 infection: Review of 3939 COVID-19 patients in China and emerging pathogenesis and therapy concepts. J Leukoc Biol 108, 17-41 (2020).
5. Bottazzi, B., Doni, A., Garlanda, C. & Mantovani, A. An integrated view of humoral innate immunity: pentraxins as a paradigm. Annu Rev Immunol 28, 157-183 (2010).
6. Holmskov, U., Thiel, S. & Jensenius, J. C. Collections and ficolins: humoral lectins of the innate immune defense. Annu Rev Immunol 21, 547-578 (2003).
7. Garlanda, C., Bottazzi, B., Magrini, E., Inforzato, A. & Mantovani, A. PTX3, a Humoral Pattern Recognition Molecule, in Innate Immunity, Tissue Repair, and Cancer. Physiol Rev 98, 623-639 (2018).
8 Reading, P. C. et al. Antiviral Activity of the Long Chain Pentraxin PTX3 against Influenza Viruses. J Immunol 180, 3391-3398 (2008).
9. Bozza, S. et al. Pentraxin 3 protects from MCMV infection and reactivation through TLR sensing pathways leading to IRF3 activation. Blood 108, 3387-3396 (2006).
10. Han, B. et al. Protective effects of long pentraxin PTX3 on lung injury in a severe acute respiratory syndrome model in mice. Lab Invest 92, 1285-1296 (2012).
11. Merad, M. & Martin, J. C. Pathological inflammation in patients with COVID-19: a key role for monocytes and macrophages. Nat Rev Immunol 20, 355-362 (2020).
12. Severe Covid, G. G. et al. Genomewide Association Study of Severe Covid-19 with Respiratory Failure. N Engl J Med 383, 1522-1534 (2020).
13. Zhang, Q. et al. Inborn errors of type I IFN immunity in patients with life-threatening COVID-19. Science 370, eabd4570 (2020).
14. Pairo-Castineira, E. et al. Genetic mechanisms of critical illness in COVID-19. Nature 591, 92-98 (2021).
15. Fajgenbaum, D. C. & June, C. H. Cytokine Storm. N Engl J Med 383, 2255-2273 (2020).
16. Brunetta, E. et al. Macrophage expression and prognostic significance of the long pentraxin PTX3 in COVID-19. Nat Immunol 22, 19-24 (2021).
17. Zeng, W. et al. Biochemical characterization of SARS-COV-2 nucleocapsid protein. Biochem Biophys Res Commun 527, 618-623 (2020).
18. Watanabe, Y., Allen, J. D., Wrapp, D., Mclellan, J. S. & Crispin, M. Site-specific glycan analysis of the SARS-COV-2 spike. Science 369, 330-333 (2020).
19. Chu, H. et al. Comparative tropism, replication kinetics, and cell damage profiling of SARS-COV-2 and SARS-COV with implications for clinical manifestations, transmissibility, and laboratory studies of COVID-19: an observational study. Lancet Microbe 1, e14-e23 (2020).
20. Desmyter, J., Melnick, J. L. & Rawls, W. E. Defectiveness of interferon production and of rubella virus interference in a line of African green monkey kidney cells (Vero). J Virol 2, 955-961 (1968).
21. Garred, P. et al. A journey through the lectin pathway of complement-MBL and beyond. Immunol Rev 274, 74-97 (2016).
22. Ip, W. K. et al. Mannose-binding lectin in severe acute respiratory syndrome coronavirus infection. J Infect Dis 191, 1697-1704 (2005).
23. Lipscombe, R. J. et al. High frequencies in African and non-African populations of independent mutations in the mannose binding protein gene. Hum Mol Genet 1, 709-715 (1992).
24. Sumiya, M. et al. Molecular basis of opsonic defect in immunodeficient children. Lancet 337, 1569-1570 (1991).

25. Madsen, H. O. et al. A new frequent allele is the missing link in the structural polymorphism of the human mannan-binding protein. Immunogenetics 40, 37-44 (1994).
26. Madsen, H. O. et al. Interplay between promoter and structural gene variants control basal serum level of mannan-binding protein. J Immunol 155, 3013-3020 (1995).
27. Fishilevich, S. et al. GeneHancer: genome-wide integration of enhancers and target genes in GeneCards. Database (Oxford) 2017 (2017).
28. Kosmicki, J. A. et al. A catalog of associations between rare coding variants and COVID-19 outcomes. medRxiv, 10.1101/2020.1110.1128.20221804 (2021).
29. Schirinzi, A. et al. Pentraxin 3: Potential prognostic role in SARS-COV-2 patients admitted to the emergency department. J Infect 82, 84-123 (2021).
30. Gao, T. et al. Highly pathogenic coronavirus N protein aggravates lung injury by MASP-2-mediated complement over-activation.medRxiv, 10.1101/2020.1103.1129.20041962 (2020).
31. Karwaciak, I., Salkowska, A., Karas, K., Dastych, J. & Ratajewski, M. Nucleocapsid and Spike Proteins of the Coronavirus SARS-COV-2 Induce IL6 in Monocytes and Macrophages-Potential Implications for Cytokine Storm Syndrome. Vaccines (Basel) 9, 54 (2021).
32. McBride, R., van Zyl, M. & Fielding, B. C. The coronavirus nucleocapsid is a multifunctional protein. Viruses 6, 2991-3018 (2014).
33. Zhou, Y. et al. A single asparagine-linked glycosylation site of the severe acute respiratory syndrome coronavirus spike glycoprotein facilitates inhibition by mannose-binding lectin through multiple mechanisms. J Virol 84, 8753-8764 (2010).
34. Lu, Q. et al. SARS-COV-2 exacerbates proinflammatory responses in myeloid cells through C-type lectin receptors and Tweety family member 2. Immunity 54, 1304-1319 e1309 (2021).
35. Lempp, F. A. et al. Lectins enhance SARS-COV-2 infection and influence neutralizing antibodies. Nature 598, 342-347 (2021).
36. Chiodo, F. et al. Novel ACE2-Independent Carbohydrate-Binding of SARS-COV-2 Spike Protein to Host Lectins and Lung Microbiota. bioRxiv, 2020.2005.2013.092478 (2020).
37. Ali, Y. M. et al. Lectin Pathway Mediates Complement Activation by SARS-COV-2 Proteins. Front Immunol 12, 714511 (2021).
38. Hsieh, M. H. et al. Human Surfactant Protein D Binds Spike Protein and Acts as an Entry Inhibitor of SARS-COV-2 Pseudotyped Viral Particles. Front Immunol 12, 641360 (2021).
39. Bayarri-Olmos, R. et al. Development of a Quantitative Assay for the Characterization of Human Collectin-11 (CL-11, CL-K1). Front Immunol 9, 2238 (2018).
40. Andreano, E. et al. SARS-COV-2 escape in vitro from a highly neutralizing COVID-19 convalescent plasma. bioRxiv, 10.1101/2020.1112.1128.424451 (2020).
41. Koch, A. et al. Acute respiratory tract infections and mannose-binding lectin insufficiency during early childhood. JAMA 285, 1316-1321 (2001).
42. Zhang, H. et al. Association between mannose-binding lectin gene polymorphisms and susceptibility to severe acute respiratory syndrome coronavirus infection. J Infect Dis 192, 1355-1361 (2005).
43. Yuan, F. F. et al. Influence of FcgammaRIIA and MBL polymorphisms on severe acute respiratory syndrome. Tissue Antigens 66, 291-296 (2005).
44. Medetalibeyoglu, A. et al. Mannose binding lectin gene 2 (rs1800450) missense variant may contribute to development and severity of COVID-19 infection. Infect Genet Evol 89, 104717 (2021).
45. Risitano, A. M. et al. Complement as a target in COVID-19? Nat Rev Immunol 20, 343-344 (2020).
46. Carvelli, J. et al. Association of COVID-19 inflammation with activation of the C5a-C5aR1 axis. Nature 588, 146-150 (2020).
47. King, C. & Sprent, J. Dual Nature of Type I Interferons in SARS-COV-2-Induced Inflammation. Trends Immunol 42, 312-322 (2021).
48. Garred, P. et al. Mannose-binding lectin (MBL) therapy in an MBL-deficient patient with severe cystic fibrosis lung disease. Pediatr Pulmonol 33, 201-207 (2002).
49. Jensenius, J. C., Jensen, P. H., McGuire, K., Larsen, J. L. & Thiel, S. Recombinant mannan-binding lectin (MBL) for therapy. Biochem Soc Trans 31, 763-767 (2003).
50. Myocardial Infarction Genetics, C. et al. Genome-wide association of early-onset myocardial infarction with single nucleotide polymorphisms and copy number variants. Nat Genet 41, 334-341 (2009).
51. Bottazzi, B. et al. Multimer formation and ligand recognition by the long pentraxin PTX3. Similarities and differences with the short pentraxins C-reactive protein and serum amyloid P component. J Biol Chem 272, 32817-32823 (1997).
52. Munthe-Fog, L. et al. The impact of FCN2 polymorphisms and haplotypes on the Ficolin-2 serum levels. Scand J Immunol 65, 383-392 (2007).
53. De Gasparo, R. et al. Bispecific IgG neutralizes SARS-COV-2 variants and prevents escape in mice. Nature 593, 424-428 (2021).
54. Consortium, T. U. UniProt: the universal protein knowledgebase in 2021. Nucleic Acids Research 49, D480-D489 (2020).
55. Sheriff, S., Chang, C. Y. & Ezekowitz, R. A. B. Human mannose-binding protein carbohydrate recognition domain trimerizes through a triple α-helical coiled-coil. Nat Struct Biol 1, 789-794 (1994).
56. Berisio, R., Vitagliano, L., Mazzarella, L. & Zagari, A. Crystal structure of the collagen triple helix model [(Pro-Pro-Gly)(l0)](3). Protein Sci 11, 262-270 (2002).
57. Ng, K. K. et al. Orientation of bound ligands in mannose-binding proteins. Implications for multivalent ligand recognition. J Biol Chem 277, 16088-16095 (2002).
58. Casalino, L. et al. Beyond Shielding: The Roles of Glycans in the SARS-COV-2 Spike Protein. ACS Cent Sci 6, 1722-1734 (2020).
59. Fu, B. et al. ALMOST: an all atom molecular simulation toolkit for protein structure determination. J Comp Chem 35, 1101-1105 (2014).
60. Cesana, D. et al. Uncovering and dissecting the genotoxicity of self-inactivating lentiviral vectors in vivo. Mol Ther 22, 774-785 (2014).
61. Stravalaci, M. et al. Control of Complement Activation by the Long Pentraxin PTX3: Implications in Age-Related Macular Degeneration. Front Pharmacol 11, 591908 (2020).
62. Follenzi, A., Ailles, L. E., Bakovic, S., Geuna, M. & Naldini, L. Gene transfer by lentiviral vectors is limited by nuclear translocation and rescued by HIV-1 pol sequences. Nat Genet 25, 217-222 (2000).
63. Scudieri, P. et al. Association of TMEM16A chloride channel overexpression with airway goblet cell metaplasia. J Physiol 590, 6141-6155 (2012).
64. Clementi, N. et al. Combined Prophylactic and Therapeutic Use Maximizes Hydroxychloroquine Anti-SARS-COV-2 Effects in vitro. Front Microbiol 11, 1704 (2020).
65. Mycroft-West, C. J. et al. Heparin Inhibits Cellular Invasion by SARS-COV-2: Structural Dependence of the Interaction of the Spike S1 Receptor-Binding Domain with Heparin. Thromb Haemost 120, 1700-1715 (2020).
66. Taliun, D. et al. Sequencing of 53,831 diverse genomes from the NHLBI TOPMed Program. Nature 590, 290-299 (2021).
67. Chang, C.C. et al. Second-generation PLINK: rising to the challenge of larger and richer datasets. Gigascience 4, 7 (2015).
68. Purcell, S. et al. PLINK: a tool set for whole-genome association and population-based linkage analyses. Am J Hum Genet 81, 559-575 (2007).
69. Browning, S. R. & Browning, B. L. Rapid and accurate haplotype phasing and missing-data inference for whole-genome association studies by use of localized haplotype clustering. Am J Hum Genet 81, 1084-1097 (2007).
70. Mantel, N. & Haenszel, W. Statistical aspects of the analysis of data from retrospective studies of disease. J Natl Cancer Inst 22, 719-748 (1959).
71. Asselta, R., Paraboschi, E. M., Mantovani, A. & Duga, S. ACE2 and TMPRSS2 variants and expression as candidates to sex and country differences in COVID-19 severity in Italy. Aging (Albany NY) 12, 10087-10098 (2020).
72. Swapan K Chatterjee, Snigdha Saha, Maria Nilda M Munoz. Molecular Pathogenesis, Immunopathogenesis and Novel Therapeutic Strategy Against COVID-19. Review. Front Mol Biosci. 2020 Aug. 11; 7:196. doi: 10.3389/fmolb.2020.00196.
73. Kristel Klaassen, Biljana Stankovic, Branka Zukic, Nikola Kotur, Vladimir Gasic, Sonja Pavlovic, Maja Stojiljkovic. Functional prediction and comparative population analysis of variants in genes for proteases and innate immunity related to SARS-COV-2 infection. Infect Genet Evol. 2020 October; 84:104498.
74. Zhu N, Zhang D, Wang W, et al. A Novel Coronavirus from Patients with Pneumonia in China, 2019. N Engl J Med 2020; 382(8): 727-33.
75. Li W, Moore M J, Vasilieva N, et al. Angiotensin-converting enzyme 2 is a functional receptor for the SARS coronavirus. Nature 2003; 426(6965): 450-4.
76. Hoffmann M, Kleine-Weber H, Schroeder S, et al. SARS-COV-2 Cell Entry Depends on ACE2 and TMPRSS2 and Is Blocked by a Clinically Proven Protease Inhibitor. Cell 2020; 181(2): 271-80 e8.

1. A method for treating and/or preventing a viral infection in an individual in need thereof caused by a pathogenic coronavirus, comprising administering to the individual in need thereof a therapeutic agent comprising:
(a) a mannose binding lectin (MBL) polypeptide, or a functional fragment, derivative, mutein or variant thereof,
(b) an MBL polypeptide having at least about 50%. 60%, 70%, 80% or 90%, or 100% identity to SEQ ID NO:2;
(c) an MBL polypeptide encoded by a nucleic acid sequence having at least about 50%. 60%. 70%, 80%, 90%, 95% or 100% to SEQ ID NO:1;
(d) a nucleic acid sequence encoding an MBL polypeptide and having at least about 50%. 60%. 70%. 80%. 90%. 95% or 100% to SEQ ID NO: 1:
(e) a nucleic acid sequence encoding the MBL polypeptide of (a) or (b);
(f) a vector having contained therein the nucleic acid of (d) or (e); or
(g) a cell having contained therein an MBL-encoding nucleic acid of (d) or (e). or the vector of (f).

2. The method of claim 1, wherein the MBL polypeptide has at least about 95% identity with the sequence of SEQ ID NO:2.

3. The method of claim 1, wherein the MBL polypeptide has at least 95% identity with a sequence encoded by SEQ ID NO:2.

4-6. (canceled)

7. The method of claim 1, wherein the nucleic acid is contained in a vector.

8. (canceled)

9. A pharmaceutical composition comprising:
(a) an antiviral therapeutic agent comprising:
(i) a mannose binding lectin (MBL) polypeptide, or a functional fragment, derivative, mutein or variant thereof.
(ii) an MBL polypeptide having at least about 50%. 60%. 70%. 80%. 90%. 95% or 100% identity to SEQ ID NO:2;
(iii) an MBL polypeptide encoded by a nucleic acid sequence having at least about 50%, 60%, 70%. 80%. 90%. 95% or 100% to SEQ ID NO:1;
(iv) a nucleic acid sequence encoding an MBL polypeptide and having at least about 50%, 60%, 70%. 80%, 90%. 95% or 100% to SEQ ID NO:1;
(v) a nucleic acid sequence encoding the MBL polypeptide of (i) or (ii);
(vi) a vector having contained therein a nucleic acid of (iv) or (v): or
(vii) a host cell having contained therein a vector of (vi) or a nucleic acid of (iv) or (v); and,
(b) at least one pharmaceutically acceptable excipient and/or carrier.

10. The pharmaceutical composition of claim 9, further comprising at least one additional antiviral therapeutic agent.

11. The method of claim 1, wherein the coronavirus infection is a COVID-19 infection, or an infection caused by severe acute respiratory syndrome coronavirus 2 (SARS-COV-2) or any variant thereof, optionally a 501 or 614 variant, or any variant comprising a mutation in a gene encoding SARS-COV-2 Spike protein.

12. The method of claim 11, wherein the COVID-19 is caused by a variant of severe acute respiratory syndrome coronavirus 2 (SARS-COV-2) selected from the group consisting of: the B.1.1.7 variant or a variant, the B.1.1.28 or P.1 variant or γ variant, the B.1.351 variant or β variant, the B.1.617.2 variant or δ variant and the Omicron (o) variant or B.1.1.529 variant.

13. The method of claim 1, wherein the individual in need thereof has a genetic polymorphism in the MBL gene leading to a low MBL production.

14. The method of claim 1, wherein the therapeutic agent is administered in the early stages of the viral infection.

15. The method molecule of claim 1, wherein the individual in need thereof has has one or more of the following Single Nucleotide Polymorphisms (SNPs) in the MBL gene: rs5030737, rs1800450, rs1800451 rs150342746, rs10824845 and rs11816263, optionally in biallelic conditions.

16. The method of claim 1, wherein the individual in need thereof has has at least one of the following haplotypes in the MBL gene: ATCGCAA, CCC, TCCCC, TCAGACC, TA, ATCCCCGCATTGA [SEQ ID N.3], AGATCCCCGCGCGTGCAACGGCTGCGGA [SEQ ID N.4], wherein each haplotype is characterized by at least the following single nucleotide polymorphisms (SNPs), wherein for haplotypes of maximum 5 SNPs all SNPs forming the haplotype are indicated, while for haplotypes including more than 5 SNPs only the first and the last SNPs are indicated:

| Haplotype | SNPs |
|---|---|
| ATCGCAA | 6 SNPs, rs11344513\|rs7071467 |
| CCC | 3 SNPs, rs17662822\|rs1159798\| |
| | rs1912619 |
| TCCCC | 5 SNPs, rs2204344\|rs12218074\| |
| | rs80035245\|rs7935712\| |
| | rs10824836 |
| TCAGACC | 5 SNPs, |
| | rs16935439\|rs147096903\| |
| | rs10824839\|rs11003267\| |
| | rs11003268 |
| TA | 2 SNPs, rs10824844\|rs10824845 |
| ATCCCCGCATTGA | 9 SNPs, rs57504125\| |
| [SEQ ID N. 3] | chr10:5308418:G:A |
| AGATCCCCGCGCGTGCAAC | 24 SNPs, rs71032688\|rs7092597 |
| GGCTGCGGA | |
| [SEQ ID N. 4] | |

17. The method of claim 1, wherein the subject to be treated has a TA haplotype which consists of the SNPs rs10824844 and rs10824845.

18. The method of claim 1, wherein the pathogenic coronavirus infection is selected from the group consisting of: Severe Acute Respiratory syndrome Coronavirus 2 (SARS-COV-2), Severe Acute Respiratory syndrome Coronavirus ((SARS)-CoV), sarbecoviruses, Severe Acute Respiratory Syndrome (SARS), Middle East Respiratory Syndrome (MERS), COVID-19, and coronavirus-associated acute respiratory distress syndrome (ARDS).

19. The method of claim 1, further comprising selecting the individual in need thereof by determining the presence or the absence in an isolated biological sample from the individual in need at least one of the following haplotypes in the MBL gene: ATCGCAA, CCC, TCCCC, TCAGACC, TA, ATCCCCGCATTGA [SEQ ID N.3], AGATCCCCGCGCGTGCAACGGCTGCGGA [SEQ ID N.4],
wherein each haplotype is characterized by at least the following SNPs, wherein for haplotypes of maximum 5 SNPs all SNPs forming the haplotype are indicated, while for haplotypes including more than 5 SNPs only the first and the last SNPs are indicated:

| Haplotype | SNPs |
|---|---|
| ATCGCAA | 6 SNPs, rs11344513\|rs7071467 |
| CCC | 3 SNPs, rs17662822\|rs1159798\| |
| | rs1912619 |
| TCCCC | 5 SNPs, rs2204344\|rs12218074\| |
| | rs80035245\|rs7935712\| |
| | rs10824836 |
| TCAGACC | 5 SNPs, |
| | rs16935439\|rs147096903\| |
| | rs10824839\|rs11003267\| |
| | rs11003268 |
| TA | 2 SNPs, rs10824844\|rs10824845 |
| ATCCCCGCATTGA | 9 SNPs, rs57504125 |
| [SEQ ID N. 3] | chr10:5308418:G:A |
| AGATCCCCGCGCGTGCAAC | 24 SNPs, rs71032688\|rs7092597 |
| GGCTGCGGA | |
| [SEQ ID N. 4] | |

wherein optionally if at least one of said haplotype is identified the subject is at risk of short-term mortality and/or of being affected by a more severe disease and/or of a poor prognosis.

20. The method of claim 1, further further comprising selecting the individual in need thereof by determining the presence or the absence in an isolated biological sample from a subject of at least one of the following SNPs in the MBL gene: rs5030737, rs1800450, rs1800451 rs150342746, rs10824845 and rs11816263, wherein optionally if at
least one of said SNPs is identified the subject is at risk of short-term mortality and/or of being affected by a more severe disease and/or of a poor prognosis.

21. The method of claim 20, wherein if two alleles of at least one of the following SNPs rs5030737, rs1800450, and rs1800451 are identified, the individual in need thereof is at risk of short-term mortality and/or of being affected by a more severe disease and/or of a poor prognosis.

20. The method of claim 1, further comprising selecting the individual in need thereof by determining the presence or the absence in an isolated biological sample from a subject of the MBL gene haplotype CCGGCC, said haplotype consisting of the following SNPs: rs1800451, rs1800450, rs5030737, rs7095891, rs7096206, rs11003125, wherein optionally if said haplotype is identified the subject is less at risk of short-term mortality and/or of being affected by a more severe disease and/or of a poor prognosis.

23-24. (canceled)

25. The pharmaceutical composition of claim 9, wherein the antiviral therapeutic agent is formulated for local administration, or formulated for pulmonary delivery, or formulated as a dry powder formulation or formulated for administration by nebulization of a liquid formulation, or formulated for systemic administration.

26. The method of claim 1, wherein the antiviral therapeutic agent is formulated for local administration, or formulated for pulmonary delivery, or formulated as a dry powder formulation or formulated for administration by nebulization of a liquid formulation, or formulated for systemic administration.
